SEQUENCE LISTING (8 sequences)

SEQ ID NO: 1 - protein, 456 aa, Homo sapiens
  PROPEP (1)...(456): Precursor protein of CP22
MILSLLFSLGGPLGWGLLGAWAQASSTSLSDLQSSRTPGVWKAEAEDTGKDPVGRNWCPYPMSKLVTLLALCKTEKFLIHSQQPCPQGAPDCQKVKVMYRMAHKPVYQVKQKVLTSLAWRCCPGYTGPNCEHHDSMAIPEPADPGDSHQEPQDGPVSFKPGHLAAVINEVEVQQEQQEHLLGDLQNDVHRVADSLPGLWKALPGNLTAASLSNDVKNVGRCCEAEAGAGAASLNASLHGLHNALFATQRSLEQHQRLFHSLFGNFQGLMEANVSLDLGKLQTMLSRKGKKQQKDLEAPRKRDKKEAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 2 - protein, 433 aa, Homo sapiens
  PEPTIDE (1)...(433): Polypeptide sequence of SEQ ID NO1 after removal of the signal peptide
ASSTSLSDLQSSRTPGVWKAEAEDTSKDPVGRNWCPYPMSKLVTLLALCKTEKFLIHSQQPCPQGAPDCQKVKVMYRMAHKPVYQVKQKVLTSLAWRCCPGYTGPNCEHHDSMAIPEPADPGDSHQEPQDGPVSFKPGHLAAVINEVEVQQEQQEHLLGDLQNDVHRVADSLPGLWKALPGNLTAASLSNDVKNVGRCCEAEAGAGAASLNASLHGLHNALFATQRSLEQHQRLFHSLFGNFQGLMEANVSLDLGKLQTMLSRKGKKQQKDLEAPRKRDKKEAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 3 - protein, 152 aa, Homo sapiens
  PEPTIDE (1)...(152): Cardiovascular disorder Plasma Polypeptide 22 (CPP 22)
EAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 4 - protein, 24 aa, Homo sapiens
  PEPTIDE (1)...(24): Tryptic peptides of CPP 22 found by MS-MS mass spectrometry in plasma samples of individuals with coronary artery disease
TPVCTTGQGSGSTATVFAMAELQK

SEQ ID NO: 5 - protein, 12 aa, Homo sapiens
  PEPTIDE (1)...(12): Tryptic peptides of CPP 22 found by MS-MS mass spectrometry in plasma samples of individuals with coronary artery disease
VWFELTQGSITK

SEQ ID NO: 6 - protein, 14 aa, Homo sapiens
  PEPTIDE (1)...(14): Tryptic peptides of CPP 22 found by MS-MS mass spectrometry in plasma samples of individuals with coronary artery disease
SLSGTAFGGFLMFK

SEQ ID NO: 7 - DNA, 1371 bp, Homo sapiens
  variation (1)...(1371): Splice variant of the Endoglyx-1 gene and comprises the cDNA coding sequence for SEQ ID NO 1
atgatcctgagcttgctgttcagccttgggggccccctgggctgggggctgctgggggcatgggcccaggcttccagtactagcctctctgatctgcagagctccaggacacctggggtctggaaggcagaggctgaggacaccggcaaggaccccgtaggacgtaactggtgcccctacccaatgtccaagctggtcaccttactagctctttgcaaaacagagaaattcctcatccactcgcagcagccgtgtccgcagggagctccagactgccagaaagtcaaagtcatgtaccgcatggcccacaagccagtgtaccaggtcaagcagaaggtgctgacctctttggcctggaggtgctgccctggctacacgggccccaactgcgagcaccacgattccatggcaatccctgagcctgcagatcctggtgacagccaccaggaacctcaggatggaccagtcagcttcaaacctggccaccttgctgcagtgatcaatgaggttgaggtgcaacaggaacagcaggaacatctgctgggagatctccagaatgatgtgcaccgggtggcagacagcctgccaggcctgtggaaagccctgcctggtaacctcacagctgcaagcctgagcaacgacgtcaagaatgtcgggcggtgctgcgaggccgaggccggggccggggccgcctccctcaacgcctcccttcacggcctccacaacgcactcttcgccactcagcgcagcttggagcagcaccagcggctcttccacagcctctttgggaacttccaagggctcatggaagccaacgtcagcctggacctggggaagctgcagaccatgctgagcaggaaagggaagaagcagcagaaagacctggaagctccccggaagagggacaagaaggaagcggagcctttggtggacatacgggtcacagggcctgtgccaggtgccttgggcgcggcgctctgggaggcaggatcccctgtggccttctatgccagcttttcagaagggacggctgccctgcagacagtgaagttcaacaccacatacatcaacattggcagcagctacttccctgaacatggctacttccgagcccctgagcgtggtgtctacctgtttgcagtgagcgttgaatttggcccagggccaggcaccgggcagctggtgtttggaggtcaccatcggactccagtctgtaccactgggcaggggagtggaagcacagcaacggtctttgccatggctgagctgcagaagggtgagcgagtatggtttgagttaacccagggatcaataacaaagagaagcctgtcgggcactgcatttgggggcttcctgatgtttaagacctga

SEQ ID NO: 8 - protein, 417 aa, Mus musculus
  PEPTIDE (1)...(417): Q8K1Z7, a murine homologue of SEQ ID NO1
MIPTLLLGFGVYLSWGLLGSWAQDPGTKFSHLNRPGMPEGWRLGAEDTSRDPIRRNWCPYQKSRLVTFVAACKTEKFLVHSQQPCPQGAPDCQGVRVMYRVAQKPVYQVQQKVLISVDWRCCPGFQGPDCQDHNPTANPEPTEPSGKLQETWDSMDGFELGHPVPEFNEIKVPQEQQEIRRLSSDVKQIGQCCEASWAASLNSSLEDLHSMLLDTQHGLRQHRQLFHNLFQNFQGLVASNISLDLGKLQAMLSKKDKKQPRGPGESRKRDKKQVVMSTDAHAKGLELWETGSPVAFYAGSSEGATALQMVKFNTTSINVGSSYFPEHGYFRAPKRGVYLFAVSITFGPGPGMGQLVFEGHHRVPVYSTEQRGGSTATTFAMVELQKGERAWFELIQGSATKGSQPGTAFGGFLMFKT

(19) United States
(12) Patent Application Publication
(10) Pub. No.: US 2007/0105169 A1
(43) Pub. Date: May 10, 2007

(54) Secreted polypeptide species associated with cardiovascular disorders

(76) Inventors: Lydie Bougueleret, Petit-Lancy (CH); Isabelle Cusin, Douvaine (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/561,292

(22) PCT Filed: Jun. 29, 2004
(86) PCT No.: PCT/EP04/07047

§ 371 Date: Aug. 8, 2006

Related U.S. Application Data
(60) Provisional application No. 60/484,153, filed on Jun. 30, 2003.

(51) Int. Cl. G01N 33/53; C07K 16/18

(52) U.S. Cl. 435/7.92; 530/388.25

(57) ABSTRACT

The invention discloses human secreted polypeptides that circulate at an increased level in the plasma of patients with cardiovascular disorders. The invention also provides methods of using compositions including the polypeptides, polynucleotides encoding them, and antibodies specific for these polypeptides, for diagnosis, prognosis, and treatment of cardiovascular disorders.

### FIELD OF THE INVENTION

The invention relates to polypeptide species secreted preferentially in individuals with cardiovascular disorders, to isolated polynucleotides encoding such polypeptides, to polymorphic variants thereof, and to the use of said nucleic acids and polypeptides or compositions thereof in detection assays, for cardiovascular disorder diagnosis, for cardiovascular disorder treatment, and for drug development.

### BACKGROUND

Cardiovascular disease is a major health risk throughout the industrialized world. Coronary Artery Disease (CAD) is characterized by atherosclerosis or hardening of the arteries. Atherosclerosis is the most prevalent of cardiovascular diseases, is the principal cause of heart attack, stroke, and gangrene of the extremities, and thereby the principle cause of death in the United States. Atherosclerosis is a complex disease involving many cell types and molecular factors (described in, for example, Ross, 1993, Nature 362: 801-809). In normal circumstances a protective response to insults to the endothelium and smooth muscle cells (SMCs) of the wall of the artery consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of many conditions that predispose an individual to accelerated development of atherosclerotic cardiovascular disease.

Atherosclerotic plaques occlude the blood vessel concerned and restrict the flow of blood, resulting in ischemia. Ischemia is a condition characterized by a lack of oxygen supply in tissues of organs due to inadequate perfusion. Such inadequate perfusion can have a number of natural causes, including atherosclerotic or restenotic lesions, anemia, or stroke. The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults.

Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis.

Extensive clinical studies have identified factors that increase the risk of cardiovascular disorders. Some of these risk factors, such as age, gender, and family history cannot be changed. Other risk factors include the following: smoking, high blood pressure, high fat and high cholesterol diet, diabetes, lack of exercise, obesity, and stress.

Fortunately, many contributing factors are controllable through lifestyle changes. The risk of cardiovascular disorders for smokers is more than twice that of non-smokers. When a person stops smoking, regardless of how much he or she may have smoked in the past, their risk of developing a disorder rapidly declines. Serum cholesterol level is directly related to prevalence of cardiovascular disorder and hypertension or high blood pressure is an important risk factor. Physical activity has been postulated to reduce the risk of developing a cardiovascular disorder through various mechanisms: it increases myocardial oxygen supply, decreases oxygen demand, and improves myocardial contraction and its electrical impulse stability. Reduced oxygen demand and myocardial work are reflected in lowered heart rate and blood pressure at rest. Physical activity also increases the diameter and dilatory capacity of coronary arteries, increases collateral artery formation, and reduces rates of progression of coronary artery atherosclerosis. Obesity and the serum fatty acids are reduced by activity.

There may be no noticeable symptoms of a cardiovascular disorder at rest, but symptoms such as chest pressure may occur with increased activity or stress. Other first signs that can appear are heartburn, nausea, vomiting, numbness, shortness of breath, heavy cold sweating, unexplained fatigue, and feelings of anxiety. The more severe symptoms of cardiovascular disorders are chest pain (angina pectoris), rhythm disturbances (arrhythmias), stroke, or heart attack (myocardial infarction). Strokes and heart attacks result from a blocked artery in the brain and heart tissue, respectively. Because symptoms vary, the tests and treatments chosen can be very different from one patient to another.

Diagnostic tests useful in determining the extent and severity of cardiovascular disorder include: electrocardiogram (EKG), stress test, nuclear scanning, coronary angiography, resting EKG, EKG Multiphase Information Diagnosis Indexes, Holter monitor, late potentials, EKG mapping, echocardiogram, Thallium scan, PET, MRI, CT, angiogram and IVUS. Additional risk factor measures and useful diagnostics are common and best applied by one of skill in the art of medicine. There are many different therapeutic approaches, depending on the seriousness of the disease. For many people, cardiovascular disorders are managed with lifestyle changes and medications. More severe diagnoses may indicate a need for surgery.

Surgical approaches to the treatment of ischemic atherosclerosis include bypass grafting, coronary angioplasty, laser angioplasty, atherectomy, endarterectomy, and percutaneous translumenal angioplasty (PCTA). The failure rate after these approaches due to restenosis, in which the occlusions recur and often become even worse, is extraordinarily high (30-50%). It appears that much of the restenosis due to further inflammation, smooth muscle accumulation, and thrombosis. Additional therapeutic approaches to cardiovascular disease have included treatments that encouraged angiogenesis in such conditions as ischemic heart and limb disease.

The non-specific nature of most CAD and cardiovascular disorder symptoms makes definitive diagnosis difficult. More quantitative diagnostic methods suffer from variability, both between individuals and between readings on a single individual. Thus, diagnostic measures must be standardized and applied to individuals with well-documented and extensive medical histories. Further, current diagnostic methods often do not reveal the underlying cause for a given observation or reading. Therefore, a therapeutic strategy based on a particular positive result likely will not address the causative problem and may even be harmful to the individual.

Methods of diagnosis that rely on nucleotide detection include genetic approaches and expression profiling. For example, genes that are known to be involved in cardiovascular disorders may be screened for mutations using common genotyping techniques such as sequencing, hybridization-based techniques, or PCR. In another example, expression from a known gene may be tracked by standard techniques including RTPCR, various hybridization-based techniques, and sequencing. These strategies often do not enable a practitioner to detect differences in mRNA processing and splicing, translation rate, mRNA stability, and posttranslational modifications such as proteolytic processing, phosphorylation, glycosylation, and amidation.

To address the current weaknesses in the diagnostic state of the art for cardiovascular disorders, the invention provides a specific polypeptide that is differentially increased in plasma from individuals with Coronary Artery Disease compared to control plasma. By providing the actual polypeptide species, differences in mRNA processing and splicing, translation rate, mRNA stability, and posttranslational modifications such as proteolytic processing, phosphorylation, glycosylation, and amidation are revealed. The polypeptides of the invention are thus described as “Cardiovascular disorder Plasma Polypeptides” or CPPs. These polypeptide sequences are described as SEQ ID NOs:1-3, and those comprising at least one of the amino acid sequences selected from the tryptic peptides of Table 1.

The present invention discloses “Cardiovascular disorder Plasma Polypeptides” (CPPs), fragments, and post-translationally modified species of CPPs that are present at a higher level in plasma obtained from individuals with Coronary Artery Disease (CAD). Thus, the CPPs of the invention represent an important diagnostic tool for determining the risk of CAD, coronary heart disease (CHD), peripheral vascular disease, cerebral ischemia (stroke), congestive heart failure, atherosclerosis, hypertension, and other cardiovascular diseases. CPPs are secreted factors and as such, are ideal candidates for protein-based therapies. For dosage modulation in a clinical setting, protein therapy is preferable to genetic therapy, which is hampered by the lack of finely regulable expression. Further, as secreted factors, the polypeptide species of the invention are easy to target, e.g., with a small molecule or protein modulator. Thus, the polypeptide species of the invention are useful for drug development and design of therapeutic strategies to prevent and treat cardiovascular disease.

### SUMMARY OF TH INVENTION

The present invention is directed to compositions related to secreted polypeptide species that are preferentially increased in plasma from individuals with a cardiovascular disorder. These polypeptide species are designated herein “Cardiovascular disorder Plasma Polypeptides,” or CPPs. Such Cardiovascular disorder Plasma Polypeptides comprise an amino acid sequence selected from the group consisting of SEQ ID NOs:1-6. SEQ ID NO:3 represents a preferred CPP (hereinafter, CPP 22). Compositions include nucleotides encoding CPPs, CPP precursors, antibodies specific for CPPs, including monoclonal antibodies and other binding compositions derived therefrom. Further included are methods of making and using these compositions. Precursors of the invention include unmodified precursors, proteolytic precursors of SEQ ID NOs:1-6, and intermediates resulting from alternative proteolytic sites in the amino acid sequences of SEQ ID NOs:1-6.

A preferred embodiment of the invention includes CPPs having a posttranslational modification, such as a phosphorylation, glycosylation, acetylation, amidation, or a C-, N- or O-linked carbohydrate group. Additionally preferred are CPPs with intra- or inter-molecular interactions, e.g., disulfide and hydrogen bonds that result in higher order structures. Also preferred are CPPs that result from differential mRNA processing or splicing. Preferably, the CPPs represent posttranslationally modified species, structural variants, or splice variants that are present in plasma from individuals with a cardiovascular disorder.

In another aspect, the invention includes CPPs comprising a sequence which is at least 75 percent identical to a sequence selected from the group consisting of SEQ ID NOs:1-6. Preferably, the invention includes polypeptides comprising at least 80 percent, and more preferably at least 85 percent, and still more preferably at least 90 percent, identity with any one of the sequences selected from SEQ ID NOs:1-6. Most preferably, the invention includes polypeptides comprising a sequence at least 95 percent identical to a sequence selected from the group consisting of SEQ ID NOs:1-6.

In another aspect, the invention includes natural variants of CPPs having a frequency in a selected population of at least two percent. More preferably, such natural variant has a frequency in a selected population of at least five percent, and still more preferably, at least ten percent. Most preferably, such natural variant has a frequency in a selected population of at least twenty percent. The selected population may be any recognized population of study in the field of population genetics. Preferably, the selected population is Caucasian, Negroid, or Asian. More preferably, the selected population is French, German, English, Spanish, Swiss, Japanese, Chinese, Irish, Korean, Singaporean, Icelandic, North American, Israeli, Arab, Turkish, Greek, Italian, Polish, Pacific Islander, Finnish, Norwegian, Swedish, Estonian, Austrian, or Indian. More preferably, the selected population is Icelandic, Saami, Finnish, French of Caucasian ancestry, Swiss, Singaporean of Chinese ancestry, Korean, Japanese, Quebecian, North American Pima Indians, Pennsylvanian Amish and Amish Mennonite, Newfoundlander, or Polynesian.

A preferred aspect of the invention provides a composition comprising an isolated CPP, i.e., a CPP free from proteins or protein isoforms having a significantly different isoelectric point or a significantly different apparent molecular weight from the CPP. The isoelectric point and molecular weight of a CPP may be indicated by affinity and size-based separation chromatography, 2-dimensional gel analysis, and mass spectrometry.

In a preferred aspect, the invention provides particular polypeptide species that comprise an amino acid sequence selected from the group consisting of SEQ ID NOs:3-6. Preferably, the particular polypeptide species further comprises contiguous amino acid sequence from SEQ ID NO:1 or 2. Preferred species are polypeptides that i) comprise an amino acid sequence of one of SEQ ID NOs:3-6; ii) appear at a higher level in plasma from individuals with a cardiovascular disorder; and iii) optionally result from proteolytic processing of the polypeptide of SEQ ID NO:1 or 2.

In an additional aspect, the invention includes modified CPPs. Such modifications include protecting/blocking groups, linkage to an antibody molecule or other cellular ligand, and detectable labels, such as an enzymatic, fluorescent, isotopic or affinity label to allow for detection and isolation of the protein. Chemical modifications may be carried out by known techniques, including but not limited, to specific chemical cleavage by cyanogen bromide, trypsin, chymotrypsin, papain, V8 protease, NaBH4, acetylation, formylation, oxidation, reduction, or metabolic synthesis in the presence of tunicamycin.

Also provided by the invention are chemically modified derivatives of the polypeptides of the invention which may provide additional advantages such as increased solubility, stability and circulating time of the polypeptide, or decreased immunogenicity (e.g., water soluble polymers such as polyethylene glycol, ethylene glycol/propylene glycol copolymers, carboxymethylcellulose, dextran, polyvinyl alcohol). The CPPs are modified at random positions within the molecule, or at predetermined positions within the molecule and may include one, two, three or more attached chemical moieties.

In another embodiment, the invention provides a method of identifying a modulator of at least one CPP biological activity comprising the steps of: i) contacting a test modulator of a CPP biological activity with the polypeptide comprising the amino acid sequence selected from the group consisting of SEQ ID NOs:1-4; ii) detecting the level of said CPP biological activity; and iii) comparing the level of said CPP biological activity to that of a control sample lacking said test modulator. Where the difference in the level of CPP protein biological activity is a decrease, the test modulator is an inhibitor of at least one CPP biological activity. Where the difference in the level of CPP biological activity is an increase, the test substance is an activator of at least one CPP biological activity.

In another aspect of the invention, a method of identifying a modulator of a cardiovascular disorder is provided, which comprises the steps of: (a) administering a candidate agent to a non-human test animal which is predisposed to be affected or which is affected by the cardiovascular disorder; (b) administering the candidate agent of (a) to a matched control non-human animal not predisposed to be affected or not being affected by the cardiovascular disorder; (c) detecting and/or quantifying the level of a polypeptide in a biological sample obtained from the non-human test animal of step (a) and from the control animal of step (b), wherein the polypeptide is selected from: (i) a polypeptide comprising the amino acid sequence of SEQ ID NO: 3; (ii) a variant, with at least 75% sequence identity, having one or more amino acid substitutions, deletions or insertions relative to the amino acid sequence shown in SEQ ID NO: 3; and (iii) a fragment of a polypeptide as defined in i) or ii) above which is a least ten amino acids long; and step (d) comparing the levels of the polypeptide of step (c); wherein a displacement of the level of the polypeptide in the biological sample obtained from the non-human test animal towards the level of the polypeptide in the biological sample obtained from the control animal indicates that the candidate agent is a modulator of the cardiovascular disorder. A preferred embodiment of the invention provides that the non-human test animal which is predisposed to be affected or which is affected by the cardiovascular disorder comprises an increased plasma level of a polypeptide selected from (i) a polypeptide comprising the amino acid sequence of SEQ ID NO: 3; (ii) a variant, with at least 75% sequence identity, having one or more amino acid substitutions, deletions or insertions relative to the amino acid sequence shown in SEQ ID NO: 3; and (iii) a fragment of a polypeptide as defined in i) or ii) above which is a least ten amino acids long.

In another aspect, the invention includes polynucleotides encoding a CPP of the invention, polynucleotides encoding a polypeptide having an amino acid sequence selected from the group consisting of SEQ ID NOs:1-6, antisense oligonucleotides complementary to such sequences, oligonucleotides complementary to CPP gene sequences for diagnostic and analytical assays (e.g., PCR, hybridization-based techniques), and vectors for expressing CPPs. A preferred polynucleotide comprises the sequence of SEQ ID NO:7.

In another aspect, the invention provides a vector comprising DNA encoding a CPP. The invention also includes host cells and transgenic nonhuman animals comprising such a vector. There is also provided a method of making a CPP or CPP precursor. One preferred method comprises the steps of (a) providing a host cell containing an expression vector as disclosed above; (b) culturing the host cell under conditions whereby the DNA segment is expressed; and (c) recovering the protein encoded by the DNA segment. Another preferred method comprises the steps of: (a) providing a host cell capable of expressing a CPP; (b) culturing said host cell under conditions that allow expression of said CPP; and (c) recovering said CPP. Within one embodiment the expression vector further comprises a secretory signal sequence operably linked to the DNA segment, the cell secretes the protein into a culture medium, and the protein is recovered from the medium. An especially preferred method of making a CPP includes chemical synthesis using standard peptide synthesis techniques, as described in the section titled “Chemical Manufacture of CPP compositions” and in Example 3.

In another aspect, the invention includes isolated antibodies specific for any of the polypeptides, peptide fragments, or peptides described above. Preferably, the antibodies of the invention are monoclonal antibodies. Further preferred are antibodies that bind to a CPP exclusively, that is, antibodies that do not recognize other polypeptides with high affinity. Anti-CPP antibodies have purification, diagnostic and therapeutic applications, particularly in treating CPP-related disorders. Preferred anti-CPP antibodies for purification and diagnosis are attached to a label group. Preferred CPP-related disorders for diagnosis include coronary artery disease (CAD), coronary heart disease (CHD), peripheral vascular disease, cerebral ischemia (stroke), congestive heart failure, atherosclerosis, hypertension, and other cardiovascular diseases. Diagnostic methods include, but are not limited to, those that employ antibodies or antibody-derived compositions specific for a CPP antigen. Diagnostic methods for detecting CPPs in specific tissue samples and biological fluids (preferably plasma), and for detecting levels of expression of CPPs in tissues, also form part of the invention. Compositions comprising one or more antibodies described above, together with a pharmaceutically acceptable carrier are also within the scope of the invention.

The invention further provides methods for diagnosis of cardiovascular disorders that comprise detecting the level of at least one CPP in a sample of body fluid, preferably blood plasma. Further included are methods of using CPP compositions, including primers complementary to CPP genes and/or messenger RNA and anti-CPP antibodies, for detecting and measuring quantities of CPPs in tissues and biological fluids, preferably plasma. These methods are also suitable for clinical screening, prognosis, monitoring the results of therapy, identifying patients most likely to respond to a particular therapeutic treatment, drug screening and development, and identifying new targets for drug treatment. Preferably, the detecting of the level of at least one CPP in a sample of body fluid is performed ex vivo.

A still further aspect of the invention relates to a method for monitoring the efficacy of a treatment of a subject having or at risk of developing a cardiovascular disorder with an agent, which comprises the steps of: (a) obtaining a pre-administration biological sample from the subject prior to administration of the agent; (b) detecting and/or quantifying the level of a polypeptide in the biological sample from said subject, wherein the polypeptide is selected from (i) a polypeptide comprising the amino acid sequence of SEQ ID NO: 3; (ii) a variant, with at least 75% sequence identity, having one or more amino acid substitutions, deletions or insertions relative to the amino acid sequence shown in SEQ ID NO: 3; and (iii) a fragment of a polypeptide as defined in i) or ii) above which is a least ten amino acids long, and which comprises steps (c) obtaining one or more post-administration biological samples from the subject; (d) detecting the level of the polypeptide in the post-administration sample or samples; (e) comparing the level of the polypeptide in the pre-administration sample with the level of the polypeptide in the post-administration sample; and (f) adjusting the administration of the agent accordingly.

The invention provides kits that may be used in the above-recited methods and that may comprise single or multiple preparations, or antibodies, together with other reagents, label groups, substrates, if needed, and directions for use. The kits may be used for diagnosis of disease, or may be assays for the identification of new diagnostic and/or therapeutic agents.

The invention further includes methods of using CPP compositions to prevent or treat disorders associated with aberrant expression or processing of CPPs of SEQ ID NOs:1-6 in an individual. Preferred CPP-related disorders include coronary artery disease (CAD), coronary heart disease (CHD), peripheral vascular disease, cerebral ischemia (stroke), congestive heart failure, atherosclerosis, hypertension, and other cardiovascular diseases. A preferred embodiment of the invention is a method of preventing or treating a CPP-related disorder in an individual comprising the steps of: determining that an individual suffers from or is at risk of a CPP-related disorder and introducing a CPP-modulating composition to said individual.

In still a further aspect, the invention includes pharmaceutical compositions and formulations comprising a polypeptide having an amino acid sequence selected from the group consisting of SEQ ID NOs:1-6, and a pharmaceutically acceptable carrier compound.

In one embodiment, Coronary Artery Disease (CAD) is defined by the appearance of at least one symptom. Such symptoms become more serious as the disease progresses. CAD is often accompanied by reduced left ventricle capacity or output. Early CAD symptoms include elevated plasma levels of cholesterol and low-density lipoprotein (especially oxidized forms), as well as platelet-rich plasma aggregations. The vascular endothelium responds to inflammation and thus formation of plaques and levels of inflammatory and fibrinogenic factors increase. In addition, CAD, or atherosclerosis, is characterized by vascular calcification and hardening of the arteries. The resulting partial occlusion of the blood vessels leads to hypertension and ischemic heart disease. Eventual complete vascular occlusion results in myocardial infarction, stroke, or gangrene.

In a preferred embodiment, detection of increased plasma levels of at least one CPP of the invention indicates an increased risk that an individual will develop CAD. Preferably, said detection indicates that an individual has at least a 1.05-fold, 1.1-fold, 1.15-fold, and more preferably at least a 1.2-fold increased likelihood of developing CAD. Alternatively, detection of increased plasma levels of at least one CPP of the invention indicates that an individual has CAD. The amount of CPP increase observed in an individual compared to a control sample will correlate with the certainty of the prediction or diagnosis of CAD. As individual plasma CPP levels will vary depending on family history and other risk factors, each will preferably be examined on a case-by-case basis. In preferred embodiments, CPP is detected in a human plasma sample by the methods of the invention. Especially preferred techniques are mass spectrometry and immunodetection. Preferably, a prediction or diagnosis of CAD is based on at least a 1.1-, 1.15-, 1.2-, 1.25-, and more preferably a 1.5-fold increase in the experimental CPP level as compared to the control.

Further aspects of the invention are also described in the specification and in the claims.

### BRIEF DESCRIPTION OF THE SEQUENCE LISTING

SEQ ID NO:1 describes the amino acid sequence of the precursor protein of the invention, whereas SEQ ID NO:2 is the polypeptide sequence after removal of the signal peptide.

SEQ ID NO:3 is the amino acid sequence of the CPP 22 polypeptide species found at an elevated level in CAD plasma from which the tryptic peptides were released.

SEQ ID NOs:4-6 are the amino acid sequences of tryptic peptides found by MS-MS mass spectrometry in plasma samples of individuals with coronary artery disease.

SEQ ID NO:7 represents a novel splice variant of the Endoglyx-1 gene and comprises the cDNA coding sequence for SEQ ID NO:1.

### BRIEF DESCRIPTION OF THE FIGURES

FIG. 1 shows the sequence of precursor protein of the invention (SEQ ID NO:1), the sequence of the mature protein (SEQ ID NO:2), the CPP 22 plasma polypeptide (SEQ ID NO:3), and peptide sequences found by MS-MS mass spectrometry in the plasma of individuals with coronary artery disease (SEQ ID NOs:4-6). The tryptic peptides observed by tandem mass spectrometry are in bold and double-underline, and the polypeptide found at an increased level in plasma of CAD patients is underlined in SEQ ID NOs:1 and 2. The signal peptide is in italics and underlined in SEQ ID NO:1. The coding sequence of the full length polypeptide is described as SEQ ID NO:7.

FIG. 2 shows the codon-amino acid alignment of the cDNA sequence of SEQ ID NO:7 with the protein sequence of SEQ ID NO:1.

FIG. 3 discloses the similarity of the protein of the invention to the murine homologue (Q8K1Z7). The two sequences were aligned using Clustal W 1.81 software. Asterisks (*) mark exact matches, while colons (:) and dots (.) mark interchangeable and similar residues, respectively.

### DETAILED DESCRIPTION OF THE INVENTION

The present invention described in detail below provides methods, compositions, and kits useful for screening, diagnosis and treatment of a cardiovascular disorder in a mammalian individual; for identifying individuals most likely to respond to a particular therapeutic treatment; for monitoring the results of cardiovascular disorder therapy; for screening CPP modulators; and for drug development. The invention also encompasses the administration of therapeutic compositions to a mammalian individual to treat or prevent cardiovascular disorders. The mammalian individual may be a non-human mammal, but is preferably human, more preferably a human adult. For clarity of disclosure, and not by way of limitation, the invention will be described with respect to the analysis of blood plasma samples. However, as one skilled in the art will appreciate, the assays and techniques described below can be applied to other biological fluid samples (e.g. cerebrospinal fluid, lymph, bile, serum, saliva or urine) or tissue samples from an individual at risk of having or developing a cardiovascular disorder. The methods and compositions of the present invention are useful for screening, diagnosis and prognosis of a living individual, but may also be used for postmortem diagnosis in an individual, for example, to identify family members who are at risk of developing the same disorder.

Definitions

As used herein, the term “nucleic acids” and “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. Throughout the present specification, the expression “nucleotide sequence” may be employed to designate indifferently a polynucleotide or a nucleic acid. More precisely, the expression “nucleotide sequence” encompasses the nucleic material itself and is thus not restricted to the sequence information (i.e. the succession of letters chosen among the four base letters) that biochemically characterizes a specific DNA or RNA molecule. Also, used interchangeably herein are terms “nucleic acids”, “oligonucleotides”, and “polynucleotides”.

An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CPP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. Using all or a portion of the nucleic acid as a hybridization probe, CPP nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

As used herein, the term “hybridizes to” is intended to describe conditions for moderate stringency or high stringency hybridization, preferably where the hybridization and washing conditions permit nucleotide sequences at least 60% homologous to each other to remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. In a preferred, non-limiting example, stringent hybridization conditions for nucleic acid interactions are as follows: the hybridization step is realized at 65° C. in the presence of 6×SSC buffer, 5× Denhardt's solution, 0.5% SDS and 100 μg/ml of salmon sperm DNA. The hybridization step is followed by four washing steps:
two washings during 5 min, preferably at 65° C. in a 2×SSC and 0.1% SDS buffer;
one washing during 30 min, preferably at 65° C. in a 2×SSC and 0.1% SDS buffer,
one washing during 10 min, preferably at 65° C. in a 0.1×SSC and 0.1% SDS buffer,
   these hybridization conditions being suitable for a nucleic acid molecule of about 20 nucleotides in length. It will be appreciated that the hybridization conditions described above are to be adapted according to the length of the desired nucleic acid, following techniques well known to the one skilled in the art, for example be adapted according to the teachings disclosed in Hames B. D. and Higgins S. J. (1985) Nucleic Acid Hybridization: A Practical Approach. Hames and Higgins Ed., IRL Press, Oxford; and Current Protocols in Molecular Biology.

“Percent homology” is used herein to refer to both nucleic acid sequences and amino acid sequences. Amino acid or nucleic acid “identity” is equivalent to amino acid or nucleic acid “homology”. To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). The length of a reference sequence aligned for comparison purposes is at least 30%, preferably at least 40%, more preferably at least 50%, even more preferably at least 60%, and even more preferably at least 70%, 80%, 90% or 95% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position. The percent homology between the two sequences is a function of the number of identical positions shared by the sequences (i.e., % homology=# of identical positions/total # of positions 100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77, the disclosures of which are incorporated herein by reference in their entireties. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the sequences of the invention. BLAST protein searches can be performed with the XBLAST program, score=50, wordlength=3 to obtain amino acid sequences homologous to the polypeptide sequences of the invention. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used. See http://www.ncbi.nlm.nih.gov, the disclosures of which are incorporated herein by reference in their entireties. Another preferred, non-limiting example of a mathematical algorithm utilized for the comparison of sequences is the algorithm of Myers and Miller, CABIOS (1989), the disclosures of which are incorporated herein by reference in their entireties. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The term “polypeptide” refers to a polymer of amino acids without regard to the length of the polymer; thus, peptides, oligopeptides, and proteins are included within the definition of polypeptide. This term also does not specify or exclude post-translational modifications of polypeptides, for example, polypeptides which include the covalent attachment of glycosyl, acetyl, phosphate, amide, lipid, carboxyl, acyl, or carbohydrate groups are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “protein” as used herein may be used synonymously with the term “polypeptide” or may refer to, in addition, a complex of two or more polypeptides which may be linked by bonds other than peptide bonds, for example, such polypeptides making up the protein may be linked by disulfide bonds. The term “protein” may also comprehend a family of polypeptides having identical amino acid sequences but different post-translational modifications, particularly as may be added when such proteins are expressed in eukaryotic hosts.

An “isolated” or “purified” protein or biologically active portion thereof is substantially free of cellular material or other contaminating proteins from the cell or tissue source from which the protein of the invention (i.e., CPP or biologically active fragment thereof) is derived, or substantially free from chemical precursors or other chemicals when chemically synthesized. The language “substantially free of cellular material” includes preparations of a protein according to the invention in which the protein is separated from cellular components of the cells from which it is isolated or recombinantly produced. In one embodiment, the language “substantially free of cellular material” includes preparations of a protein according to the invention having less than about 30% (by dry weight) of protein other than the protein of the invention (also referred to herein as a “contaminating protein”), more preferably less than about 20% of protein other than the protein according to the invention, still more preferably less than about 10% of protein other than the protein according to the invention, and most preferably less than about 5% of protein other than the protein according to the invention. When the protein according to the invention or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of a protein of the invention in which the protein is separated from chemical precursors or other chemicals which are involved in the synthesis of the protein. In one embodiment, the language “substantially free of chemical precursors or other chemicals” includes preparations of a protein of the invention having less than about 30% (by dry weight) of chemical precursors or non-protein chemicals, more preferably less than about 20% chemical precursors or non-protein chemicals, still more preferably less than about 10% chemical precursors or non-protein chemicals, and most preferably less than about 5% chemical precursors or non-protein chemicals.

The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

The term “Cardiovascular disorder Plasma Polypeptide” or “CPP” refers to a polypeptide comprising the sequence described by any one of SEQ ID NOs:1-6. Such polypeptide may be post-translationally modified as described herein. CPPs may also contain other structural or chemical modifications such as disulfide linkages or amino acid side chain interactions such as hydrogen and amide bonds that result in complex secondary or tertiary structures. CPPs also include mutant polypeptides, such as deletion, addition, swap, or truncation mutants, fusion polypeptides comprising such polypeptides, and polypeptide fragments of at least three, but preferably 8, 10, 12, 15, or 21 contiguous amino acids of the sequence of SEQ ID NOs:1-6. Further included are CPP proteolytic precursors and intermediates of the sequence selected from the group consisting of SEQ ID NOs:1-6. The invention embodies polypeptides encoded by the nucleic acid sequences of CPP genes or CPP mRNA species, preferably human CPP genes and mRNA species, including isolated CPPs consisting of, consisting essentially of, or comprising the sequence of SEQ ID NOs:1-6. Preferred CPPs have a sequence comprising the sequence of SEQ ID NO:3. Preferred nucleotide species comprise the coding sequence of SEQ ID NO:7. Further preferred is the fragment of SEQ ID NO:7 encoding SEQ ID NO:3. Preferred CPPs retain at least one biological activity of CPPs of SEQ ID NOs:1-6.

The term “biological activity” as used herein refers to any single function carried out by a CPP. These include but are not limited to: (1) indicating that an individual has or will have a cardiovascular disorder; (2) circulating through the bloodstream of individuals with a cardiovascular disorder; (3) antigenicity, or the ability to bind an anti-CPP specific antibody; (4) immunogenicity, or the ability to generate an anti-CPP specific antibody; (5) forming intermolecular amino acid side chain interactions such as hydrogen, amide, or preferably disulfide links; (6) interaction with a CPP target molecule, or in particular, an endothelial cell surface; and (7) undergoing posttranslational processing, in particular, specific proteolysis or glycosylation.

As used herein, a “CPP modulator” or “CPP 22 modulator” is a molecule (e.g., polynucleotide, polypeptide, small molecule, or antibody) that is capable of modulating (i.e., increasing or decreasing) either the expression or the biological activity of the CPPs of the invention. A CPP modulator that enhances CPP expression or activity is described as a CPP activator or agonist. Conversely, a CPP modulator that represses CPP expression or activity is described as a CPP inhibitor or antagonist. Preferably, CPP modulators increase/decrease the expression or activity by at least 5, 10, or 20%. CPP inhibitors include anti-CPP antibodies, fragments thereof, antisense polynucleotides, and molecules characterized by screening assays, as described herein. CPP agonists include polynucleotide expression vectors and molecules characterized by screening assays as described herein.

A “CPP-related disorder” or “CPP-related disease” describes a cardiovascular disorder. Preferred disorders include coronary artery disease (CAD), coronary heart disease (CHD), peripheral vascular disease, cerebral ischemia (stroke), congestive heart failure, atherosclerosis, hypertension, and other cardiovascular diseases. Preferably, the likelihood that an individual will develop or already has such a disorder is indicated by higher than normal plasma levels of at least one CPP.

Another aspect of the invention pertains to anti-CPP antibodies. The term “antibody” as used herein refers to immunoglobulin molecules and immunologically active portions of immunoglobulin molecules, i.e., molecules that contain an antigen-binding site which specifically binds (immunoreacts with) an antigen, such as CPP, or a biologically active fragment or homologue thereof. Preferred antibodies bind to a CPP exclusively and do not recognize other polypeptides with high affinity. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind a CPP, or a biologically active fragment or homologue thereof. The term “monoclonal antibody” or “monoclonal antibody composition”, as used herein, refers to a population of antibody molecules that contain only one species of an antigen-binding site capable of immunoreacting with a particular epitope of a CPP. A monoclonal antibody composition thus typically displays a single binding affinity for a particular CPP with which it immunoreacts. Preferred CPP antibodies are attached to a label group.

As used herein, a “label group” is any compound that, when attached to a polynucleotide or polypeptide (including antibodies), allows for detection or purification of said polynucleotide or polypeptide. Label groups may be detected or purified directly or indirectly by a secondary compound, including an antibody specific for said label group. Useful label groups include radioisotopes (e.g., ³²P, ³⁵S, ³H, ¹²⁵I), fluorescent compounds (e.g., 5-bromodesoxyuridin, umbelliferone, fluorescein, fluorescein isothiocyanate, rhodamine, dichlorotriazinylamine fluorescein, dansyl chloride, phycoerythrin acetylaminofluorene, digoxigenin), luminescent compounds (e.g., luminol, GFP, luciferin, aequorin), enzymes or enzyme co-factor detectable labels (e.g., peroxidase, luciferase, alkaline phosphatase, galactosidase, or acetylcholinesterase), or compounds that are recognized by a secondary factor such as strepavidin, GST, or biotin. Preferably, a label group is attached to a polynucleotide or polypeptide in such a way as to not interfere with the biological activity of the polynucleotide or polypeptide.

Radioisotopes may be detected by direct counting of radioemission, film exposure, or by scintillation counting, for example. Enzymatic labels may be detected by determination of conversion of an appropriate substrate to product, usually causing a fluorescent reaction. Fluorescent and luminescent compounds and reactions may be detected by, e.g., radioemission, fluorescent microscopy, fluorescent activated cell sorting, or a luminometer.

As used herein with respect to antibodies, an antibody is said to “selectively bind” or “specifically bind” to a target if the antibody recognizes and binds the target of interest but does not substantially recognize and bind other molecules in a sample, e.g., a biological sample, which includes the target of interest.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, “effective amount” describes the amount of an agent, preferably a CPP or CPP modulator of the invention, sufficient to have a desired effect. For example, an anticardiovascular disorder effective amount is the amount of an agent required to reduce a symptom of a cardiovascular disorder in an individual by at least 1, 2, 5, 10, 15, or preferably 25%. The term may also describe the amount of an agent required to ameliorate a cardiovascular disorder-caused symptom in an individual. Common symptoms of cardiovascular disorders include: chest pressure, heartburn, nausea, vomiting, numbness, shortness of breath, heavy cold sweating, unexplained fatigue, and feelings of anxiety. The more severe symptoms of cardiovascular disorders are chest pain (angina pectoris), rhythm disturbances (arrhythmias), stroke, or heart attack. The effective amount for a particular patient may vary depending on such factors as the diagnostic method of the symptom being measured, the state of the condition being treated, the overall health of the patient, method of administration, and the severity of side-effects.

CPPs of the Invention

The Cardiovascular disorder Plasma Polypeptides (CPPs) of the invention are described in the sequence listing as SEQ ID NOs:1-6. SEQ ID NO:3 is the sequence of the CAD plasma peptide released from SEQ ID NOs:1 and 2. CPPs comprising an amino acid sequence selected from the group consisting of SEQ ID NOs:2-6 are secreted and circulate at an elevated level in blood plasma of individuals that have or are at risk of developing a cardiovascular disorder.

Further included CPPs are polypeptides comprising an amino acid sequence of SEQ ID NO:3-6. Preferably, such CPPs also comprise additional amino acids from SEQ ID NO:1 or 2. Such additional amino acids are fused in frame with the selected sequence to form contiguous amino acid sequence from the protein of SEQ ID NO:1 or 2.

The CPPs of the invention, and in particular the polypeptide species with the amino acid sequence of SEQ ID NO:3 (CPP 22), is a previously unreported plasma protein. The protein of SEQ ID NO:3 is released from a precursor that is a novel splice variant of the human Endoglyx-1 gene (Q9H8L6). Interestingly, the level of the CPPs of the invention is dramatically increased (9.7-fold) in the plasma of individuals suffering from cardiovascular disorders. As such, the CPPs of the invention provide a useful diagnostic tool, wherein an increased level of a CPP indicates an increased risk of developing, or the presence of, a cardiovascular disorder. Further, CPPs are useful for drug design and in therapeutic strategies for prevention and treatment of cardiovascular disorders.

Endoglyx-1 was cloned from vascular endothelial cells and characterized by Christian, et al. (J. Biol. Chem. 276:48588-48595 (2001)). Endoglyx is present as part of a multimeric complex associated with the extracellular matrix of vascular endothelial cells. The N terminus comprises an EMI (Elastin Microfibril Interacting) domain with extensive disulfide-mediated structure linked to a C terminal C1q domain by a coiled coil region. The protein is glycosylated and shares homology with other glucosaminoglycan-binding proteins such as EMILIN and Multimerin. The presently described novel splice variant (SEQ ID NO:1) lacks much of the coiled coil structure Endoglyx-1, but retains the EMI and C1q domains. The preferred sequence of the invention, CPP 22 from SEQ ID NO:3, comprises solely the C-terminal C1q domain of the EndoGlyx-1 protein. This domain is proposed to interact with glycosaminoglycans such as heparin and heparan sulfate.

Full length Endoglyx-1 protein is normally associated with the endothelial membrane, thus the presence of the polypeptides in blood plasma is unexpected. The present inventors have discovered elevated levels of a peptide species encoded by the Endoglyx gene in the plasma of CAD patients. Thus, the current invention provides an important diagnostic tool for determining an individual's risk of developing a cardiovascular disorder.

The role of heparin in Coronary Artery Disease has been studied for many years, and heparin has been suggested to have a preventive role against Coronary Artery Disease (see for example, Ragazzi and Chinellato, Gen Pharmacol., 1995, July; 26(4):697-701). The CPP 22 protein of the invention, as a molecule circulating in plasma with a potential to bind glycosaminoglycans and in particular heparin, therefore represents a candidate therapeutic molecule for the treatment and prophylaxis of Coronary Artery Diseases.

The terms “Cardiovascular disorder Plasma Polypeptide” and “CPP” are used herein to embrace any and all of the peptides, polypeptides and proteins of the present invention. Also forming part of the invention are polypeptides encoded by the polynucleotides of the invention, as well as fusion polypeptides comprising such polypeptides. The invention embodies CPPs from humans, including isolated or purified CPPs consisting of, consisting essentially of, or comprising an amino acid sequence selected from the group consisting of SEQ ID NOs:1-6. Further included are unmodified precursors, proteolytic precursors and intermediates of the sequence selected from the group consisting of SEQ ID NOs:1-6.

The present invention embodies isolated, purified, and recombinant polypeptides comprising a contiguous span of at least 3 amino acids, preferably at least 8 to 10 amino acids, with a CPP biological activity. In preferred embodiments the contiguous stretch of amino acids comprises the site of a mutation or functional mutation, including a deletion, addition, swap or truncation of the amino acids in the CPP sequence. The invention also concerns the polypeptide encoded by the CPP nucleotide sequences of the invention, or a complementary sequence thereof or a fragment thereof.

One aspect of the invention pertains to isolated CPPs, and biologically active portions thereof, as well as polypeptide fragments suitable for use as immunogens to raise anti-CPP antibodies. In one embodiment, native CPP peptides can be isolated from plasma, cells or tissue sources by an appropriate purification scheme using standard protein purification techniques. In another embodiment, CPPs are produced by recombinant DNA techniques. Alternative to recombinant expression, a CPP can be synthesized chemically using peptide synthesis techniques, as described in the section titled “Chemical Manufacture of CPP compositions” and in Example 3.

Typically, biologically active portions comprise a domain or motif with at least one activity of a CPP. CPP domains include disufide bonds, an EMI domain, and a C1q domain. A biologically active CPP may, for example, comprise at least 1, 2, 3, or 5 amino acid changes from the sequence selected from the group consisting of SEQ ID NOs:1-6, or comprise at least 1%, 2%, 3%, 5%, 8%, 10% or 15% change in amino acids from the sequence selected from the group consisting of SEQ ID NOs:1-6.

Characterization of CPPs

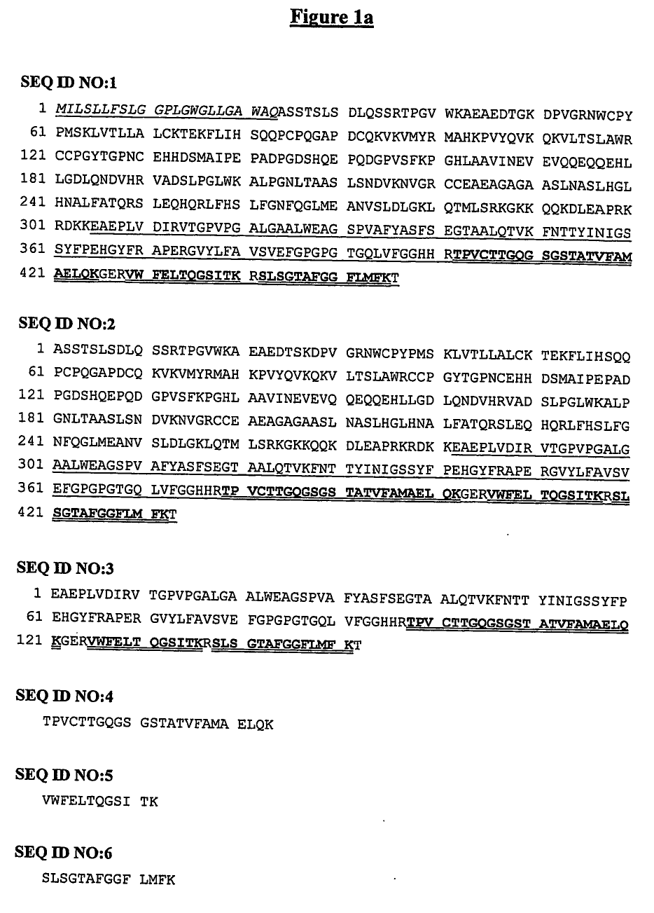

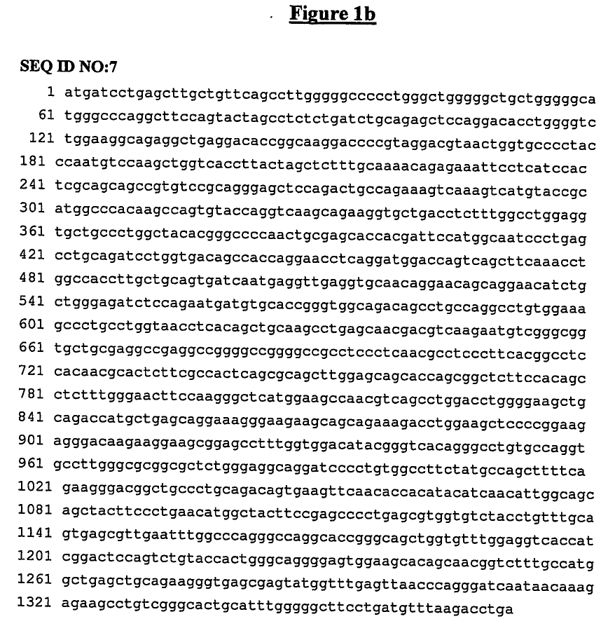

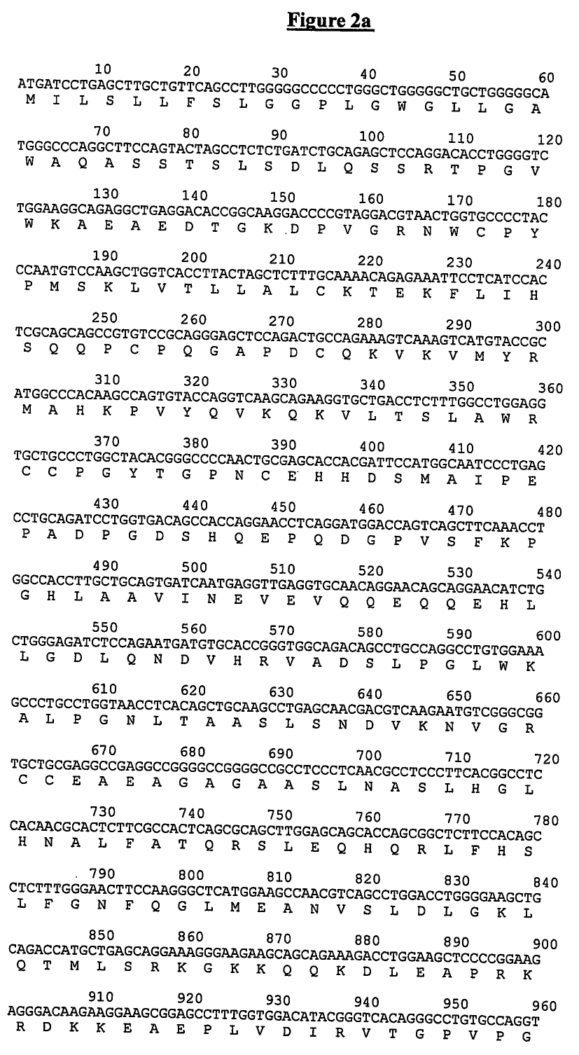

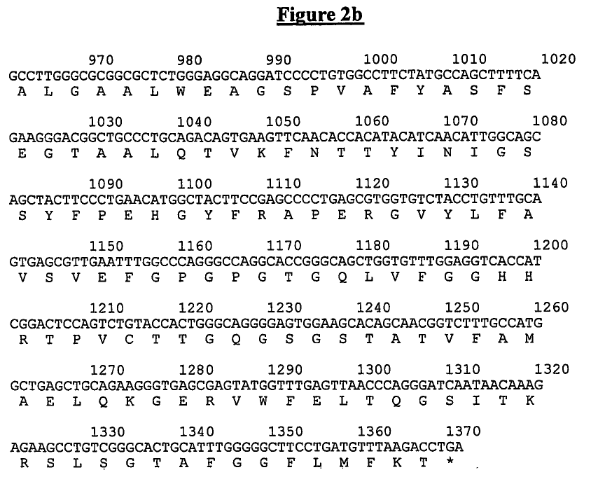

The polypeptides of the invention, CPPs, are defined by the tryptic peptides of SEQ ID NOs:4-6 (FIG. 1 and Table 1). These peptides were isolated from the plasma of Coronary Artery Disease patients and characterized according to the MicroProt® method, as described in Example 1. SEQ ID NO:3 represents the polypeptide species found at an elevated level in CAD plasma from which the tryptic peptides were released.

The CPPs of the invention are all less than or around 20 kD in molecular weight, as the plasma sample is first separated based on molecular weight. Higher molecular weight polypeptide species are separated and characterized by a different method. As described in Example 1, the plasma sample is subjected to a number of chromatography separations. Details about these chromatography methods are given in Example 1.

The first separation is on a cation exchange chromatography column, which is eluted with increasing salt concentration. Eighteen fractions are collected. The CEX column in Table 1 lists which fraction contained each tryptic peptide, as well as its elution conditions. Separation by cation exchange provides an indication of the overall positive charge of a polypeptide species. Cation exchange is followed by a reverse phase HPLC separation. The RP1 column in Table 1 lists in which of the 30 fractions each tryptic peptide eluted, as well as its elution conditions. Separation by reverse phase provides an indication of the overall hydrophobicity of a polypeptide species. The last two digits of the column labeled Run Number indicate which of the 24 eluted fractions from the second reverse phase HPLC separation contained the tryptic peptides (see Example 1). Olav scores reflect, among other things, the strength of the experimental MS-MS signal over noise, as detected by the MS-MS data identification software, and thus give an indication of the protein concentration in the sample.

The ratio of protein levels in CAD versus control plasma samples is calculated by two methods. The first method calculates the CAD/Control ratio by the number of fractions from each sample containing the CPPs. The result is 7.5 (see Table 1). Alternatively, and more accurately, the Olav scores obtained for each peptide in the mass spectrometry data analysis software are used to give a weighted ratio. This result is 9.7, indicating that the CPPs of the invention are present at 9.7 times the level in CAD plasma compared to control plasma. As such, the CPPs provide a useful diagnostic tool, wherein an increased level of a CPP indicates an increased risk of developing, or the presence of, a cardiovascular disorder.

TABLE 1

| | | | | | | | |
|---|---|---|---|---|---|---|---|
| Peptide Sequence | CEX | Salt | RP1 | % B | Run Number | Proteome | Olav Score |

| | | | | | | | |
|---|---|---|---|---|---|---|---|
| SLSGTAFGGFLMFK | 2 | 75 | 22 | 60.56 | 121271_09 | Control | 50.33 |
| SLSGTAFGGFLMFK | | | | | 121271_06 | Control | 49.44 |
| SLSGTAFGGFLMFK | | | | | 121271_08 | Control | 32.2 |
| SLSGTAFGGFLMFK | | | | | 121271_06 | Control | 50.91 |
| VWFELTQGSITK | | | 23 | 62.48 | 121279_05 | Control | 23.73 |
| SLSGTAFGGFLMFK | | | | | 121279_04 | Control | 20.3 |
| SLSGTAFGGFLMFK | | | | | 121279_04 | Control | 37.52 |
| SLSGTAFGGFLMFK | | | | | 121279_05 | Control | 41.59 |
| VWFELTQGSITK | 5 | 75 | 29 | 74 | 117742_06 | Control | 26.41 |
| VWFELTQGSITK | | | | | 117742_07 | Control | 41.51 |
| VWFELTQGSITK | | | | | 117742_05 | Control | 22.35 |
| SLSGTAFGGFLMFK | | | | | 117742_07 | Control | 18.04 |
| VWFELTQGSITK | 2 | 75 | 19 | 54.8 | 84230_08 | CAD | 29.5 |
| SLSGTAFGGFLMFK | | | | | 84230_08 | CAD | 47.22 |
| TPVCTTGQGSGSTATVFAMAELQK | | | 20 | 56.71 | 84234_07 | CAD | 63.89 |
| SLSGTAFGGFLMFK | | | | | 84234_07 | CAD | 40.75 |
| TPVCTTGQGSGSTATVFAMAELQK | | | 21 | 58.64 | 84250_07 | CAD | 73.41 |
| VWFELTQGSITK | | | | | 130111_22 | CAD | 24.98 |
| VWFELTQGSITK | | | | | 84250_07 | CAD | 43.52 |
| VWFELTQGSITK | | | | | 84250_12 | CAD | 12.04 |
| VWFELTQGSITK | | | | | 130111_23 | CAD | 38.91 |
| VWFELTQGSITK | | | | | 130111_24 | CAD | 22.86 |
| SLSGTAFGGFLMFK | | | | | 130111_19 | CAD | 21.21 |
| SLSGTAFGGFLMFK | | | | | 130111_23 | CAD | 37.55 |
| SLSGTAFGGFLMFK | | | | | 130111_22 | CAD | 15.42 |
| SLSGTAFGGFLMFK | | | | | 130111_23 | CAD | 13.9 |
| SLSGTAFGGFLMFK | | | | | 130111_22 | CAD | 33.41 |
| SLSGTAFGGFLMFK | | | | | 84250_07 | CAD | 30.73 |
| SLSGTAFGGFLMFK | | | | | 84250_07 | CAD | 37.91 |
| TPVCTTGQGSGSTATVFAMAELQK | | | 22 | 60.56 | 84182_07 | CAD | 76.08 |
| TPVCTTGQGSGSTATVFAMAELQK | | | | | 84182_20 | CAD | 30.8 |
| TPVCTTGQGSGSTATVFAMAELQK | | | | | 84182_12 | CAD | 50.04 |
| TPVCTTGQGSGSTATVFAMAELQK | 2 | 75 | 22 | 60.56 | 84182_24 | CAD | 50.48 |
| TPVCTTGQGSGSTATVFAMAELQK | | | | | 84182_08 | CAD | 78.12 |
| TPVCTTGQGSGSTATVFAMAELQK | | | | | 84182_10 | CAD | 72.44 |
| TPVCTTGQGSGSTATVFAMAELQK | | | | | 84182_16 | CAD | 52.88 |
| TPVCTTGQGSGSTATVFAMAELQK | | | | | 84182_18 | CAD | 54.71 |
| TPVCTTGQGSGSTATVFAMAELQK | | | | | 84182_23 | CAD | 36.85 |
| TPVCTTGQGSGSTATVFAMAELQK | | | | | 84182_17 | CAD | 47.43 |
| TPVCTTGQGSGSTATVFAMAELQK | | | | | 84182_11 | CAD | 83.02 |
| TPVCTTGQGSGSTATVFAMAELQK | | | | | 84182_09 | CAD | 49.7 |
| TPVCTTGQGSGSTATVFAMAELQK | | | | | 84182_06 | CAD | 98.73 |
| TPVCTTGQGSGSTATVFAMAELQK | | | | | 84182_21 | CAD | 49.06 |
| VWFELTQGSITK | | | | | 84182_07 | CAD | 44.84 |
| VWFELTQGSITK | | | | | 84182_11 | CAD | 56.72 |
| VWFELTQGSITK | | | | | 84182_19 | CAD | 32.03 |
| VWFELTQGSITK | | | | | 84182_09 | CAD | 46.77 |
| VWFELTQGSITK | | | | | 84182_12 | CAD | 25.76 |
| VWFELTQGSITK | | | | | 84182_08 | CAD | 58.26 |
| VWFELTQGSITK | | | | | 84182_06 | CAD | 58.39 |
| VWFELTQGSITK | | | | | 84182_10 | CAD | 44.02 |
| VWFELTQGSITK | | | | | 84182_07 | CAD | 53.6 |
| VWFELTQGSITK | | | | | 84182_13 | CAD | 34.27 |
| SLSGTAFGGFLMFK | | | | | 84182_12 | CAD | 45.85 |
| SLSGTAFGGFLMFK | | | | | 84182_07 | CAD | 47.35 |
| SLSGTAFGGFLMFK | | | | | 84182_08 | CAD | 62.76 |
| SLSGTAFGGFLMFK | | | | | 84182_21 | CAD | 43.02 |
| SLSGTAFGGFLMFK | | | | | 84182_19 | CAD | 41.96 |
| SLSGTAFGGFLMFK | | | | | 84182_10 | CAD | 49.59 |
| SLSGTAFGGFLMFK | | | | | 84182_17 | CAD | 28.45 |
| SLSGTAFGGFLMFK | | | | | 84182_20 | CAD | 32.74 |
| SLSGTAFGGFLMFK | | | | | 84182_07 | CAD | 46 |
| SLSGTAFGGFLMFK | | | | | 84182_12 | CAD | 58.89 |
| SLSGTAFGGFLMFK | | | | | 84182_13 | CAD | 40.9 |
| SLSGTAFGGFLMFK | | | | | 84182_24 | CAD | 24.53 |
| SLSGTAFGGFLMFK | 2 | 75 | 22 | 60.56 | 84182_09 | CAD | 60.09 |
| SLSGTAFGGFLMFK | | | | | 84182_18 | CAD | 39.43 |
| SLSGTAFGGFLMFK | | | | | 84182_06 | CAD | 66.16 |
| SLSGTAFGGFLMFK | | | | | 84182_16 | CAD | 43.61 |
| SLSGTAFGGFLMFK | | | | | 84182_06 | CAD | 48.52 |
| SLSGTAFGGFLMFK | | | | | 84182_12 | CAD | 24.83 |
| TPVCTTGQGSGSTATVFAMAELQK | 2 | 75 | 23 | 62.48 | 84198_12 | CAD | 58.33 |
| TPVCTTGQGSGSTATVFAMAELQK | | | | | 84198_06 | CAD | 89.15 |
| TPVCTTGQGSGSTATVFAMAELQK | | | | | 84198_07 | CAD | 74.28 |
| TPVCTTGQGSGSTATVFAMAELQK | | | | | 84198_11 | CAD | 57.07 |
| TPVCTTGQGSGSTATVFAMAELQK | | | | | 84198_08 | CAD | 64.65 |
| VWFELTQGSITK | | | | | 84198_11 | CAD | 30.51 |
| VWFELTQGSITK | | | | | 84198_11 | CAD | 13.87 |
| SLSGTAFGGFLMFK | | | | | 84198_08 | CAD | 47.61 |
| SLSGTAFGGFLMFK | | | | | 84198_11 | CAD | 40.78 |
| SLSGTAFGGFLMFK | | | | | 84198_07 | CAD | 29.8 |
| SLSGTAFGGFLMFK | | | | | 84198_12 | CAD | 22.95 |
| SLSGTAFGGFLMFK | | | | | 84198_06 | CAD | 23.18 |
| VWFELTQGSITK | | | 24 | 64.4 | 84214_07 | CAD | 52.57 |
| VWFELTQGSITK | | | | | 84214_08 | CAD | 57.63 |
| VWFELTQGSITK | | | | | 84214_08 | CAD | 56.92 |
| TPVCTTGQGSGSTATVFAMAELQK | | | 25 | 68.25 | 84226_07 | CAD | 95.58 |
| VWFELTQGSITK | | | | | 84226_08 | CAD | 50.95 |
| VWFELTQGSITK | | | | | 84226_07 | CAD | 64.76 |
| VWFELTQGSITK | | | | | 84226_08 | CAD | 67.38 |
| VWFELTQGSITK | | | | | 84226_05 | CAD | 39.82 |
| SLSGTAFGGFLMFK | | | | | 84226_08 | CAD | 23.62 |
| SLSGTAFGGFLMFK | 3 | 75 | 24 | 64.4 | 89830_08 | CAD | 29.1 |
| SLSGTAFGGFLMFK | | | | | 89830_08 | CAD | 15.96 |
| TPVCTTGQGSGSTATVFAMAELQK | 4 | 75 | 23 | 62.48 | 88133_07 | CAD | 15.54 |
| SLSGTAFGGFLMFK | | | | | 88133_10 | CAD | 11.72 |
| SLSGTAFGGFLMFK | | | | | 88133_09 | CAD | 19.78 |
| VWFELTQGSITK | 5 | 75 | 25 | 68.25 | 100678_07 | CAD | 51.38 |
| SLSGTAFGGFLMFK | | | | | 100678_03 | CAD | 11.67 |
| SLSGTAFGGFLMFK | | | | | 100678_07 | CAD | 65.56 |
| SLSGTAFGGFLMFK | | | | | 100678_04 | CAD | 29.13 |
| SLSGTAFGGFLMFK | | | | | 100678_08 | CAD | 10.9 |
| SLSGTAFGGFLMFK | | | | | 100678_07 | CAD | 18.7 |

Number of Control Fractions: 12
Number of CAD Fractions: 90
CAD/Control: 7.5
Control Olav Score: 414.33
CAD Olav Score: 4017.74
CAD/Control: 9.70
CPP Nucleic Acids

One aspect of the invention pertains to purified or isolated nucleic acid molecules that encode CPPs or biologically active portions thereof as further described herein, as well as nucleic acid fragments thereof. Said nucleic acids may be used for example in therapeutic and diagnostic methods and in drug screening assays as further described herein.

An object of the invention is a purified, isolated, or recombinant nucleic acid coding for a CPP, complementary sequences thereto, and fragments thereof. A preferred polynucleotide sequence comprises the coding sequence of SEQ ID NO:7. Also preferred are fragments of SEQ ID NO:7 encoding the polypeptides of the invention. The invention also pertains to a purified or isolated nucleic acid comprising a polynucleotide having at least 95% nucleotide identity with a polynucleotide coding for a CPP, advantageously 99% nucleotide identity, preferably 99.5% nucleotide identity and most preferably 99.8% nucleotide identity with a polynucleotide coding for a CPP, or a sequence complementary thereto or a biologically active fragment thereof. Another object of the invention relates to purified, isolated or recombinant nucleic acids comprising a polynucleotide that hybridizes, under the stringent hybridization conditions defined herein, with a polynucleotide coding for a CPP, or a sequence complementary thereto or a variant thereof or a biologically active fragment thereof.

In another preferred aspect, the invention pertains to purified or isolated nucleic acid molecules that encode a portion or variant of a CPP, wherein the portion or variant displays a CPP biological activity. Preferably said portion or variant is a portion or variant of a naturally occurring CPP or precursor thereof.

Another object of the invention is a purified, isolated, or recombinant nucleic acid encoding a CPP comprising, consisting essentially of, or consisting of the amino acid sequence selected from the group of SEQ ID NOs:1-6, or fragments thereof, wherein the isolated nucleic acid molecule encodes one or more motifs such as a complement C1q motif or a disulfide bond.

The nucleotide sequence determined from the cloning of the CPP-encoding gene allows for the generation of probes and primers designed for use in identifying and/or cloning other CPPs (e.g. sharing the novel functional domains), as well as CPP homologues from other species.

A nucleic acid fragment encoding a “biologically active portion of a CPP” can be prepared by isolating a portion of a nucleotide sequence coding for a CPP, which encodes a polypeptide having a CPP biological activity, expressing the encoded portion of the CPP (e.g., by recombinant expression in vitro or in vivo) and assessing the activity of the encoded portion of the CPP.

The invention further encompasses nucleic acid molecules that differ from the CPP nucleotide sequences of the invention due to degeneracy of the genetic code and encode the same CPPs of the invention.

In addition to the CPP nucleotide sequences described above, it will be appreciated by those skilled in the art that DNA sequence polymorphisms that lead to changes in the amino acid sequences of the CPPs may exist within a population (e.g., the human population). Such genetic polymorphism may exist among individuals within a population due to natural allelic variation. Such natural allelic variations can typically result in 1-5% variance in the nucleotide sequence of a CPP-encoding gene or nucleic acid sequence.

Nucleic acid molecules corresponding to natural allelic variants and homologues of the CPP nucleic acids of the invention can be isolated based on their homology to the CPP nucleic acids disclosed herein using the cDNAs disclosed herein, or a portion thereof, as a hybridization probe according to standard hybridization techniques under stringent hybridization conditions.

It will be appreciated that the invention comprises polypeptides having an amino acid sequence encoded by any of the polynucleotides of the invention.

Uses of CPP Nucleic Acids

Polynucleotide sequences (or the complements thereof) encoding CPPs have various applications, including uses as hybridization probes, in chromosome and gene mapping, and in the generation of antisense RNA and DNA. In addition, CPP-encoding nucleic acids are useful as targets for pharmaceutical intervention, e.g. for the development of DNA vaccines, and for the preparation of CPPs by recombinant techniques, as described herein. The polynucleotides described herein, including sequence variants thereof, can be used in diagnostic assays. Accordingly, diagnostic methods based on detecting the presence of such polynucleotides in body fluids or tissue samples are a feature of the present invention. Examples of nucleic acid based diagnostic assays in accordance with the present invention include, but are not limited to, hybridization assays, e.g., in situ hybridization, and PCR-based assays. Polynucleotides, including extended length polynucleotides, sequence variants and fragments thereof, as described herein, may be used to generate hybridization probes or PCR primers for use in such assays. Such probes and primers will be capable of detecting polynucleotide sequences, including genomic sequences that are similar, or complementary to, the CPP polynucleotides described herein.

The invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair form a perfectly match duplex with the complementary strand of the same polynucleotide, and ii) the primers of a pair form such perfectly matched duplexes at sites on the polynucleotide that separated by a distance of between 10 and 2500 nucleotides. Preferably, the annealing temperature of each primer of a pair to its respective complementary sequence is substantially the same.

Hybridization probes derived from polynucleotides of the invention can be used, for example, in performing in situ hybridization on tissue samples, such as fixed or frozen tissue sections prepared on microscopic slides or suspended cells. Briefly, a labeled DNA or RNA probe is allowed to bind its DNA or RNA target sample in the tissue section on a prepared microscopic, under controlled conditions. Generally, dsDNA probes consisting of the DNA of interest cloned into a plasmid or bacteriophage DNA vector are used for this purpose, although ssDNA or ssRNA probes may also be used. Probes are generally oligonucleotides between about 15 and 40 nucleotides in length. Alternatively, the probes can be polynucleotide probes generated by PCR random priming primer extension or in vitro transcription of RNA from plasmids (riboprobes). These latter probes are typically several hundred base pairs in length. The probes can be labeled by any of a number of label groups and the particular detection method will correspond to the type of label utilized on the probe (e.g., autoradiography, X-ray detection, fluorescent or visual microscopic analysis, as appropriate). The reaction can be further amplified in situ using immunocytochemical techniques directed against the label of the detector molecule used, such as an antibody directed to a fluorescein moiety present on a fluorescently labeled probe. Specific labeling and in situ detection methods can be found, for example, in Howard, G. C., Ed., Methods in Nonradioactive Detection, Appleton & Lange, Norwalk, Conn., (1993), herein incorporated by reference.

Hybridization probes and PCR primers may also be selected from the genomic sequences corresponding to the full-length proteins identified in accordance with the present invention, including promoter, enhancer elements and introns of the gene encoding the naturally occurring polypeptide. Nucleotide sequences encoding a CPP can also be used to construct hybridization probes for mapping the gene encoding that CPP and for the genetic analysis of individuals. Individuals carrying variations of, or mutations in the gene encoding a CPP of the present invention may be detected at the DNA level by a variety of techniques. Nucleic acids used for diagnosis may be obtained from a patient's cells, including, for example, tissue biopsy and autopsy material. Genomic DNA may be used directly for detection or may be amplified enzymatically by using PCR (Saiki, et al. Nature 324:163-166 (1986)) prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype. Point mutations can be identified by hybridizing amplified DNA to radiolabeled RNA of the invention or alternatively, radiolabeled antisense DNA sequences of the invention. Sequence changes at specific locations may also be revealed by nuclease protection assays, such as RNase and S1 protection or the chemical cleavage method (e.g. Cotton, et al., Proc. Natl. Acad. Sci. USA 85:4397-4401 (1985)), or by differences in melting temperatures. “Molecular beacons” (Kostrkis L. G. et al., Science 279:1228-1229 (1998)), hairpin-shaped, single-stranded synthetic oligonucleotides containing probe sequences which are complementary to the nucleic acid of the present invention, may also be used to detect point mutations or other sequence changes as well as monitor expression levels of CPPs.

Oligonucleotide and Antisense Compounds

Oligonucleotides of the invention, including PCR primers and antisense compounds, are synthesized by conventional means on a commercially available automated DNA synthesizer, e.g. an Applied Biosystems (Foster City, Calif.) model 380B, 392 or 394 DNA/RNA synthesizer, or like instrument. Preferably, phosphoramidite chemistry is employed, e.g. as disclosed in the following references: Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like. For therapeutic use, nuclease resistant backbones are preferred. Many types of modified oligonucleotides are available that confer nuclease resistance, e.g. phosphorothioate, phosphorodithioate, phosphoramidate, or the like, described in many references, e.g. phosphorothioates: Stec et al, U.S. Pat. No. 5,151,510; Hirschbein, U.S. Pat. No. 5,166,387; Bergot, U.S. Pat. No. 5,183,885; phosphoramidates: Froehler et al, International application PCT/US90/03138; and for a review of additional applicable chemistries: Uhlmann and Peyman (cited above). The length of the antisense oligonucleotides has to be sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

Primers and Probes

Primers and probes of the invention can be prepared by any suitable method, including, for example, cloning and restriction of appropriate sequences and direct chemical synthesis by a method such as the phosphodiester method of Narang S A et al (Methods Enzymol 1979; 68:90-98), the phosphodiester method of Brown E L et al (Methods Enzymol 1979; 68:109-151), the diethylphosphoramidite method of Beaucage et al (Tetrahedron Lett 1981, 22: 1859-1862) and the solid support method described in EP 0 707 592, the disclosures of which are incorporated herein by reference in their entireties.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047. If desired, the probe may be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group.

Any of the polynucleotides of the present invention can be labeled, if desired, by incorporating any label group known in the art to be detectable by spectroscopic, photochemical, biochemical, immunochemical, or chemical means. Additional examples include non-radioactive labeling of nucleic acid fragments as described in Urdea et al. (Nucleic Acids Research. 11:4937-4957, 1988) or Sanchez-Pescador et al. (J. Clin. Microbiol. 26(10):1934-1938, 1988). In addition, the probes according to the present invention may have structural characteristics such that they allow the signal amplification, such structural characteristics being, for example, branched DNA probes as those described by Urdea et al (Nucleic Acids Symp. Ser. 24:197-200, 1991) or in the European patent No. EP 0225807 (Chiron).

A label can also be used to capture the primer, so as to facilitate the immobilization of either the primer or a primer extension product, such as amplified DNA, on a solid support. A capture label is attached to the primers or probes and can be a specific binding member which forms a binding pair with the solid's phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in CPP-encoding genes or mRNA using other techniques.

Any of the nucleic acids, polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member attached to the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The nucleic acids, polynucleotides, primers and probes of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Any polynucleotide provided herein may be attached in overlapping areas or at random locations on a solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092, the disclosures of which are incorporated herein by reference in their entireties.

Methods for Obtaining Variant Nucleic Acids and Polypeptides

In addition to naturally-occurring allelic variants of the CPP sequences that may exist in the population, the skilled artisan will appreciate that changes can be introduced by mutation into the nucleotide sequences coding for CPPs, thereby leading to changes in the amino acid sequence of the encoded CPPs, with or without altering the functional ability of the CPPs.

Several types of variants are contemplated including 1) one in which one or more of the amino acid residues are substituted with a conserved or non-conserved amino acid residue and such substituted amino acid residue may or may not be one encoded by the genetic code, or 2) one in which one or more of the amino acid residues includes a substituent group, or 3) one in which the mutated CPP is fused with another compound, such as a compound to increase the half-life of the polypeptide (for example, polyethylene glycol), or 4) one in which the additional amino acids are fused to the CPP, such as a leader, a signal or anchor sequence, a sequence which is employed for purification of the CPP, or sequence from a precursor protein. Such variants are deemed to be within the scope of those skilled in the art.

For example, nucleotide substitutions leading to amino acid substitutions can be made in the sequences that do not substantially change the biological activity of the protein. An amino acid residue can be altered from the wild-type sequence encoding a CPP, or a biologically active fragment or homologue thereof without altering the biological activity. In general, amino acid residues that are shared among the CPPs of the present invention are predicted to be less amenable to alteration.

In another aspect, the invention pertains to nucleic acid molecules encoding CPPs that contain changes in amino acid residues that result in increased biological activity, or a modified biological activity. In another aspect, the invention pertains to nucleic acid molecules encoding CPPs that contain changes in amino acid residues that are essential for a CPP biological activity. Such CPPs differ in amino acid sequence from SEQ ID NOs:1-6 and display reduced activity, or essentially lack one or more CPP biological activities.

Mutations, substitutions, additions, or deletions can be introduced into any of SEQ ID NOs:1-6, by standard techniques, such as site-directed mutagenesis and PCR-mediated mutagenesis. For example, conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a CPP, or a biologically active fragment or homologue thereof may be replaced with another amino acid residue from the same side chain family. Alternatively, in another embodiment, mutations can be introduced randomly along all or part of a CPP coding sequence, such as by saturation mutagenesis, and the resultant mutants can be screened for CPP biological activity to identify mutants that retain activity. Following mutagenesis of the nucleotide encoding one of SEQ ID NOs:1-6, the encoded protein can be expressed recombinantly and the activity of the protein can be determined in any suitable assay, for example, as provided herein.

The invention also provides CPP chimeric or fusion proteins. As used herein, a CPP “chimeric protein” or “fusion protein” comprises a CPP of the invention or fragment thereof, operatively linked or fused in frame to a non-CPP polypeptide sequence. In a preferred embodiment, a CPP fusion protein comprises at least one biologically active portion of a CPP. In another preferred embodiment, a CPP fusion protein comprises at least two biologically active portions of a CPP. For example, in one embodiment, the fusion protein is a GST-CPP fusion protein in which CPP domain sequences are fused to the C-terminus of the GST sequences. Such fusion proteins can facilitate the purification of recombinant CPPs. In another embodiment, the fusion protein is a CPP containing a heterologous signal sequence at its N-terminus, for example, to allow for a desired cellular localization in a certain host cell. In yet another embodiment, the fusion is a CPP biologically active fragment and an immunoglobulin molecule. Such fusion proteins are useful, for example, to increase the valency of CPP binding sites. For example, a bivalent CPP binding site may be formed by fusing biologically active CPP fragments to an IgG Fc protein.

The CPP fusion proteins of the invention can be incorporated into pharmaceutical compositions and administered to a subject in vivo. Moreover, the CPP fusion proteins of the invention can be used as immunogens to produce anti-CPP antibodies in a subject, to purify CPP or CPP ligands and in screening assays to identify CPP modulators.

Furthermore, isolated fragments of CPPs can also be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a CPP of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments with a CPP biological activity, for example, by microinjection assays or in vitro protein binding assays. In an illustrative embodiment, peptidyl portions of a CPP, such as a CPP target binding region, can be tested for CPP activity by expression as thioredoxin fusion proteins, each of which contains a discrete fragment of the CPP (see, for example, U.S. Pat. Nos. 5,270,181 and 5,292,646; and PCT publication WO94/02502, the disclosures of which are incorporated herein by reference).

In addition, libraries of fragments of a CPP coding sequence can be used to generate a variegated population of CPP fragments for screening and subsequent selection of variants of a CPP. In one embodiment, a library of coding sequence fragments can be generated by treating a double stranded PCR fragment of CPP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the CPP.

Modified CPPs can be used for such purposes as enhancing therapeutic or prophylactic efficacy, or stability (e.g., ex vivo shelf life and resistance to proteolytic degradation in vivo). Such modified peptides, when designed to retain at least one activity of the naturally occurring form of the protein, are considered functional equivalents of the CPP described in more detail herein. Such modified peptide can be produced, for instance, by amino acid substitution, deletion, or addition.

Whether a change in the amino acid sequence of a peptide results in a functional CPP homolog can be readily determined by assessing at least one CPP biological activity of the variant peptide. Peptides in which more than one replacement has taken place can readily be tested in the same manner.

This invention further contemplates a method of generating sets of combinatorial mutants of the presently disclosed CPPs, as well as truncation and fragmentation mutants, and is especially useful for identifying potential variant sequences which are functional in binding to a CPP target protein but differ from a wild-type form of the protein by, for example, efficacy, potency and/or intracellular half-life. One purpose for screening such combinatorial libraries is, for example, to isolate novel CPP homologs with altered biological activity, when compared with the wild-type protein, or alternatively, possessing novel activities all together. For example, mutagenesis can give rise to CPP homologs which have intracellular half-lives dramatically different than the corresponding wild-type protein. The altered protein can be rendered either more stable or less stable to proteolytic degradation, or cellular processes which result in destruction of, or otherwise inactivation of, a CPP. Such CPP homologs, and the genes which encode them, can be utilized to alter the envelope of expression for a particular recombinant CPP by modulating the half-life of the recombinant protein. For instance, a short half-life can give rise to more transient biological effects associated with a particular recombinant CPP and, when part of an inducible expression system, can allow tighter control of recombinant protein levels within a cell and in circulating plasma. As above, such proteins, and particularly their recombinant nucleic acid constructs, can be used in gene therapy protocols.

In an illustrative embodiment of this method, the amino acid sequences for a population of CPP homologs or other related proteins are aligned, preferably to promote the highest homology possible. Such a population of variants can include, for example, CPP homologs from one or more species, or CPP homologs from the same species but which differ due to mutation. Amino acids which appear at each position of the aligned sequences are selected to create a degenerate set of combinatorial sequences. There are many ways by which the library of potential CPP homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential CPP sequences. The synthesis of degenerate oligonucleotides is well known in the art (see for example. Narang, S A (1983) Tetrahedron 393; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477). Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815). The disclosures of the above references are incorporated herein by reference in their entireties.

Alternatively, other forms of mutagenesis can be utilized to generate a combinatorial library, particularly where no other naturally occurring homologs have yet been sequenced. For example, CPP homologs can be generated and isolated from a library by screening using, for example, alanine scanning mutagenesis and the like (Ruf et al. (1994) Biochemistry 33:1565-1572; Wang et al. (1994) J Biol. Chem. 269:3095-3099; Balint et al. (1993) Gene 137:109-118; Grodberg et al. (1993) Eur. J Biochem. 218:597-601; Nagashima et al. (1993) J Biol. Chem. 268:2888-2892; Lowman et al. (1991) Biochemistry 30:10832-10838; and Cunningham et al. (1989) Science 244:1081-1085), by linker scanning mutagenesis (Gustin et al. (1993) Virology 193:653-660; Brown et al. (1992) Mol. Cell Biol. 12:2644 2652; McKnight et al. (1982) Science 232:316); by saturation mutagenesis (Meyers et al. (1986) Science 232:613); by PCR mutagenesis (Leung et al. (1989) Method Cell Mol Biol 1:1-19); or by random mutagenesis (Miller et al. (1992) A Short Course in Bacterial Genetics, CSHL Press, Cold Spring Harbor, N.Y.; and Greener et al. (1994) Strategies in Mol Biol 7:32-34, the disclosures of which are incorporated herein by reference in their entireties).

A further method exploits automatic protein design to generate protein libraries for screening and optimization of the sequence of a protein of the invention. See, for example, U.S. Pat. No. 6,403,312, disclosure of which is incorporated herein by reference. Briefly, a primary library is generated using computational processing based on the sequence and structural characteristics of the CPP. Generally speaking, the goal of the computational processing is to determine a set of optimized protein sequences that result in the lowest energy conformation of any possible sequence. However, a plurality of sequences that are not the global minimum may have low energies and be useful. Thus, a primary library comprising a rank ordered list of sequences, generally in terms of theoretical quantitative stability, is generated. These sequences may be used to synthesize or express peptides displaying an extended half-life or stabilized interactions with CPP binding compounds and proteins.

A wide range of techniques are known in the art for screening gene products of combinatorial libraries made by point mutations, as well as for screening cDNA libraries for gene products having a certain property. Such techniques will be generally adaptable for rapid screening of the gene libraries generated by the combinatorial mutagenesis of CPPs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Each of the illustrative assays described below are amenable to high throughput analysis as necessary to screen large numbers of degenerate CPP sequences created by combinatorial mutagenesis techniques. In one screening assay, the candidate gene products are displayed on the surface of a cell or viral particle, and the ability of particular cells or viral particles to bind a CPP target molecule (for example a modified peptide substrate) via this gene product is detected in a “panning assay”. For instance, the gene library can be cloned into the gene for a surface membrane protein of a bacterial cell, and the resulting fusion protein detected by panning (Ladner et al., WO 89/06630; Fuchs et al. (1991) BioTechnology 9:1370-1371, and Goward et al. (1992) TIBS 18:136 140). In a similar fashion, fluorescently labeled CPP target can be used to score for potentially functional CPP homologs. Cells can be visually inspected and separated under a fluorescence microscope, or, where the morphology of the cell permits, separated by a fluorescence-activated cell sorter.

In an alternate embodiment, the gene library is expressed as a fusion protein on the surface of a viral particle. For instance, in the filamentous phage system, foreign peptide sequences can be expressed on the surface of infectious phage, thereby conferring two significant benefits. First, since these phages can be applied to affinity matrices at very high concentrations, a large number of phage can be screened at one time. Second, since each infectious phage displays the combinatorial gene product on its surface, if a particular phage is recovered from an affinity matrix in low yield, the phage can be amplified by another round of infection. The group of almost identical E. coli filamentous phages M13, fd, and fl are most often used in phage display libraries, as either of the phage gIII or gVIII coat proteins can be used to generate fusion proteins without disrupting the ultimate packaging of the viral particle (Ladner et al. PCT publication WO 90/02909; Garrard et al., PCT publication WO 92/09690; Marks et al. (1992) J Biol. Chem. 267:16007-16010; Griffiths et al. (1993) EMBO J 12:725-734; Clackson et al. (1991) Nature 352:624-628; and Barbas et al. (1992) PNAS 89:4457 4461, the disclosures of which are incorporated herein by reference in their entireties). In an illustrative embodiment, the recombinant phage antibody system (RPAS, Pharmacia Catalog number 27-9400-01) can be easily modified for use in expressing CPP combinatorial libraries, and the CPP phage library can be panned on immobilized CPP target molecule (glutathione immobilized CPP target-GST fusion proteins). Successive rounds of phage amplification and panning can greatly enrich for CPP homologs which retain an ability to bind a CPP target and which can subsequently be screened further for biological activities in automated assays, in order to distinguish between agonists and antagonists.

The invention also provides for identification and reduction to functional minimal size of the CPP functional domains, to generate mimetics, e.g. peptide or non-peptide agents, which are able to disrupt binding of a polypeptide of the present invention with a CPP target molecule. Thus, such mutagenic techniques as described above are also useful to map the determinants of CPPs participating in protein-protein interactions involved in, for example, binding to a CPP target protein. To illustrate, the critical residues of a CPP involved in molecular recognition of the CPP target can be determined and used to generate CPP target-13P-derived peptidomimetics that competitively inhibit binding of the CPP to the CPP target. For instance, non hydrolysable peptide analogs of such residues can be generated using retro-inverse peptides (e.g., see U.S. Pat. Nos. 5,116,947 and 5,219,089; and Pallai et al. (1983) Int J Pept Protein Res 21:84-92), benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides. Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), P-turn dipeptide cores (Nagai et al. (1985) Tetrahedron Lett 26:647; and Sato et al. (1986) J Chem Soc Perkin Trans 1: 123 1), and P-aminoalcohols (Gordon et al. (1985) Biochem Biophys Res Commun 126:419; and Dann et al. (1986) Biochem Biophys Res Commun 134:71, the disclosures of which are incorporated herein by reference in their entireties).

Chemical Manufacture of CPP Compositions

Peptides of the invention are synthesized by standard techniques (e.g. Stewart and Young, Solid Phase Peptide Synthesis, 2nd Ed., Pierce Chemical Company, Rockford, Ill., 1984). Preferably, a commercial peptide synthesizer is used, e.g. Applied Biosystems, Inc. (Foster City, Calif.) model 430A, and polypeptides of the invention may be assembled from multiple, separately synthesized and purified, peptide in a convergent synthesis approach, e.g. Kent et al, U.S. Pat. No. 6,184,344 and Dawson and Kent, Annu. Rev. Biochem., 69: 923-960 (2000). Peptides of the invention may be assembled by solid phase synthesis on a cross-linked polystyrene support starting from the carboxyl terminal residue and adding amino acids in a stepwise fashion until the entire peptide has been formed. The following references are guides to the chemistry employed during synthesis: Schnolzer et al, Int. J. Peptide Protein Res., 40: 180-193 (1992); Merrifield, J. Amer. Chem. Soc., Vol. 85, pg. 2149 (1963); Kent et al., pg 185, in Peptides 1984, Ragnarsson, Ed. (Almquist and Weksell, Stockholm, 1984); Kent et al., pg. 217 in Peptide Chemistry 84, Izumiya, Ed. (Protein Research Foundation, B. H. Osaka, 1985); Merrifield, Science, Vol. 232, pgs. 341-347 (1986); Kent, Ann. Rev. Biochem, Vol. 57, pgs. 957-989 (1988), and references cited in these latter two references.

Preferably, chemical synthesis of polypeptides of the invention is carried out by the assembly of peptide fragments by native chemical ligation, as described by Dawson et al, Science, 266: 776-779 (1994) and Kent et al, U.S. Pat. No. 6,184,344. Briefly, in the approach a first peptide fragment is provided with an N-terminal cysteine having an unoxidized sulfhydryl side chain, and a second peptide fragment is provided with a C-terminal thioester. The unoxidized sulfhydryl side chain of the N-terminal cysteine is then condensed with the C-terminal thioester to produce an intermediate peptide fragment which links the first and second peptide fragments with a β-aminothioester bond. The β-aminothioester bond of the intermediate peptide fragment then undergoes an intramolecular rearrangement to produce the peptide fragment product which links the first and second peptide fragments with an amide bond. Preferably, the N-terminal cysteine of the internal fragments is protected from undesired cyclization and/or concatenation reactions by a cyclic thiazolidine protecting group as described below. Preferably, such cyclic thiazolidine protecting group is a thioprolinyl group.

Peptide fragments having a C-terminal thioester may be produced as described in the following references, which are incorporated by reference: Kent et al, U.S. Pat. No. 6,184,344; Tam et al, Proc. Natl. Acad. Sci., 92: 12485-12489 (1995); Blake, Int. J. Peptide Protein Res., 17: 273 (1981); Canne et al, Tetrahedron Letters, 36: 1217-1220 (1995); Hackeng et al, Proc. Natl. Acad. Sci., 94: 7845-7850 (1997); or Hackeng et al, Proc. Natl. Acad. Sci., 96: 10068-10073 (1999). Preferably, the method described by Hackeng et al (1999) is employed. Briefly, peptide fragments are synthesized on a solid phase support (described below) typically on a 0.25 mmol scale by using the in situ neutralization/HBTU activation procedure for Boc chemistry disclosed by Schnolzer et al, Int. J. Peptide Protein Res., 40: 180-193 (1992), which reference is incorporated herein by reference. (HBTU is 2-1H-benzotriazol-1-yl)-1,1,3,3-tetramethyluronium hexafluorophosphate and Boc is tert-butoxycarbonyl). Each synthetic cycle consists of N-Boc removal by a 1- to 2-minute treatment with neat TFA, a 1-minute DMF flow wash, a 10- to 20-minute coupling time with 1.0 mmol of preactivated Boc-amino acid in the presence of DIEA, and a second DMF flow wash. (TFA is trifluoroacetic acid, DMF is N,N-dimethylformamide, and DIEA is N,N-diisopropylethylamine). N^{α}-Boc-amino acids (1.1 mmol) are preactivated for 3 minutes with 1.0 mmol of HBTU (0.5 M in DMF) in the presence of excess DIEA (3 mmol). After each coupling step, yields are determined by measuring residual free amine with a conventional quantitative ninhydrin assay, e.g. as disclosed in Sarin et al, Anal. Biochem., 117: 147-157 (1981). After coupling of Gln residues, a DCM flow wash is used before and after deprotection by using TFA, to prevent possible high-temperature (TFA/DMF)-catalyzed pyrrolidone formation. After chain assembly is completed, the peptide fragments are deprotected and cleaved from the resin by treatment with anhydrous HF for 1 hour at 0° C. with 4% p-cresol as a scavenger. The imidazole side-chain 2,4-dinitrophenyl (dnp) protecting groups remain on the His residues because the dnp-removal procedure is incompatible with C-terminal thioester groups. However, dnp is gradually removed by thiols during the ligation reaction. After cleavage, peptide fragments are precipitated with ice-cold diethylether, dissolved in aqueous acetonitrile, and lyophilized.

Thioester peptide fragments described above are preferably synthesized on a trityl-associated mercaptopropionic acid-leucine (TAMPAL) resin, made as disclosed by Hackeng et al (1999), or comparable protocol. Briefly, N^{α}-Boc-Leu (4 mmol) is activated with 3.6 mmol of HBTU in the presence of 6 mmol of DIEA and coupled for 16 minutes to 2 mmol of p-methylbenzhydrylamine (MBHA) resin, or the equivalent. Next, 3 mmol of S-trityl mercaptopropionic acid is activated with 2.7 mmol of HBTU in the presence of 6 mmol of DIEA and coupled for 16 minutes to Leu-MBHA resin. The resulting TAMPAL resin can be used as a starting resin for polypeptide-chain assembly after removal of the trityl protecting group with two 1-minute treatments with 3.5% triisopropylsilane and 2.5% H₂O in TFA. The thioester bond can be formed with any desired amino acid by using standard in situ-neutralization peptide coupling protocols for 1 hour, as disclosed in Schnolzer et al (cited above). Treatment of the final peptide fragment with anhydrous HF yields the C-terminal activated mercaptopropionic acid-leucine (MPAL) thioester peptide fragments.

Preferably, thiazolidine-protected thioester peptide fragment intermediates are used in native chemical ligation under conditions as described by Hackeng et al (1999), or like conditions. Briefly, 0.1 M phosphate buffer (pH 8.5) containing 6 M guanidine, 4% (vol/vol) benzylmercaptan, and 4% (vol/vol) thiophenol is added to dry peptides to be ligated, to give a final peptide concentration of 1-3 mM at about pH 7, lowered because of the addition of thiols and TFA from the lyophilized peptide. Preferably, the ligation reaction is performed in a heating block at 37° C. and is periodically vortexed to equilibrate the thiol additives. The reaction may be monitored for degree of completion by MALDI-MS or HPLC and electrospray ionization MS.

After a native chemical ligation reaction is completed or stopped, the N-terminal thiazolidine ring of the product is opened by treatment with a cysteine deprotecting agent, such as O-methylhydroxylamine (0.5 M) at pH 3.5-4.5 for 2 hours at 37° C., after which a 10-fold excess of Tris-(2-carboxyethyl)-phosphine is added to the reaction mixture to completely reduce any oxidizing reaction constituents prior to purification of the product by conventional preparative HPLC. Preferably, fractions containing the ligation product are identified by electrospray MS, are pooled, and lyophilized.

After the synthesis is completed and the final product purified, the final polypeptide product may be refolded by conventional techniques, e.g. Creighton, Meth. Enzymol., 107: 305-329 (1984); White, Meth. Enzymol., 11: 481-484 (1967); Wetlaufer, Meth. Enzymol., 107: 301-304 (1984); and the like. Preferably, a final product is refolded by air oxidation by the following, or like: The reduced lyophilized product is dissolved (at about 0.1 mg/mL) in 1 M guanidine hydrochloride (or like chaotropic agent) with 100 mM Tris, 10 mM methionine, at pH 8.6. After gentle overnight stirring, the re-folded product is isolated by reverse phase HPLC with conventional protocols.

Recombinant Expression Vectors and Host Cells

The polynucleotide sequences described herein can be used in recombinant DNA molecules that direct the expression of the corresponding polypeptides in appropriate host cells. Because of the degeneracy in the genetic code, other DNA sequences may encode the equivalent amino acid sequence, and may be used to clone and express the CPPs. Codons preferred by a particular host cell may be selected and substituted into the naturally occurring nucleotide sequences, to increase the rate and/or efficiency of expression. The nucleic acid (e.g., cDNA or genomic DNA) encoding the desired CPP may be inserted into a replicable vector for cloning (amplification of the DNA), or for expression. The polypeptide can be expressed recombinantly in any of a number of expression systems according to methods known in the art (Ausubel, et al., editors, Current Protocols in Molecular Biology, John Wiley & Sons, New York 1990). Appropriate host cells include yeast, bacteria, archebacteria, fungi, and insect and animal cells, including mammalian cells, for example primary cells, including stern cells, including, but not limited to bone marrow stem cells. More specifically, these include, but are not limited to, microorganisms such as bacteria transformed with recombinant bacteriophage, plasmid or cosmid DNA expression vectors, and yeast transformed with yeast expression vectors. Also included, are insect cells infected with a recombinant insect virus (such as baculovirus), and mammalian expression systems. The nucleic acid sequence to be expressed may be inserted into the vector by a variety of procedures. In general, DNA is inserted into an appropriate restriction endonuclease site using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The CPPs of the present invention are produced by culturing a host cell transformed with an expression vector containing a nucleic acid encoding a CPP, under the appropriate conditions to induce or cause expression of the protein. The conditions appropriate for CPP expression will vary with the choice of the expression vector and the host cell, as ascertained by one skilled in the art. For example, the use of constitutive promoters in the expression vector may require routine optimization of host cell growth and proliferation, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

A host cell strain may be chosen for its ability to modulate the expression of the inserted sequences or to process the expressed protein in the desired fashion. Such modifications of the protein include, but are not limited to, glycosyl, acetyl, phosphate, amide, lipid, carboxyl, acyl, or carbohydrate groups. Post-translational processing, which cleaves a “prepro” form of the protein, may also be important for correct insertion, folding and/or function. By way of example, host cells such as CHO, HeLa, BHK, MDCK, 293, W138, etc. have specific cellular machinery and characteristic mechanisms for such post-translational activities and may be chosen to ensure the correct modification and processing of the introduced, foreign protein. Of particular interest are Drosophila melanogaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, human cells and other primary cells.

The nucleic acid encoding a CPP must be “operably linked” by placing it into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” DNA sequences are contiguous, and, in the case of a secretory leader or other polypeptide sequence, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Promoter sequences encode either constitutive or inducible promoters. The promoters may be either naturally occurring promoters or hybrid promoters. Hybrid promoters, which combine elements of more than one promoter, are also known in the art, and are useful in the present invention. The expression vector may comprise additional elements, for example, the expression vector may have two replication systems, thus allowing it to be maintained in two organisms, for example in mammalian or insect cells for expression and in a procaryotic host for cloning and amplification. Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells. Further, for integrating expression vectors, the expression vector contains at least one sequence homologous to the host cell genome, and preferably, two homologous sequences which flank the expression construct. The integrating vector may be directed to a specific locus in the host cell by selecting the appropriate homologous sequence for inclusion in the vector. Constructs for integrating vectors are well known in the art. In an additional embodiment, a heterologous expression control element may be operably linked with the endogenous gene in the host cell by homologous recombination (described in U.S. Pat. Nos. 6,410,266 and 6,361,972, disclosures of which are hereby incorporated by reference in their entireties). This technique allows one to regulate expression to a desired level with a chosen control element while ensuring proper processing and modification of CPP endogenously expressed by the host cell. Useful heterologous expression control elements include but are not limited to CMV immediate early promoter, the HSV thymidine kinase promoter, the early and late SV40 promoters, the promoters of retroviral LTRs, such as those of the Rous Sarcoma Virus (RSV), and metallothionein promoters.

Preferably, the expression vector contains a selectable marker gene to allow the selection of transformed host cells. Selection genes are well known in the art and will vary with the host cell used. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available for from complex media, e.g., the gene encoding D-alanine racemase for Bacilli.

Host cells transformed with a nucleotide sequence encoding a CPP may be cultured under conditions suitable for the expression and recovery of the encoded protein from cell culture. The protein produced by a recombinant cell may be secreted, membrane-bound, or contained intracellularly depending on the sequence and/or the vector used. As will be understood by those of skill in the art, expression vectors containing polynucleotides encoding the CPP can be designed with signal sequences which direct secretion of the CPP through a prokaryotic or eukaryotic cell membrane. The desired CPP may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the CPP-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published Apr. 4, 1990), or the signal described in WO 90113646 published Nov. 15, 1990. In mammalian cell expression, mammalian signal sequences may be used to direct secretion of the protein, such as signal sequences from secreted polypeptides of the same or related species, as well as viral secretory leaders. According to the expression system selected, the coding sequence is inserted into an appropriate vector, which in turn may require the presence of certain characteristic “control elements” or “regulatory sequences.” Appropriate constructs are known generally in the art (Ausubel, et al., 1990) and, in many cases, are available from commercial suppliers such as Invitrogen (San Diego, Calif.), Stratagene (La Jolla, Calif.), Gibco BRL (Rockville, Md.) or Clontech (Palo Alto, Calif.).

Expression in Bacterial Systems

Transformation of bacterial cells may be achieved using an inducible promoter such as the hybrid lacZ promoter of the “BLUESCRIPT” Phagemid (Stratagene) or “pSPORT1” (Gibco BRL). In addition, a number of expression vectors may be selected for use in bacterial cells to produce cleavable fusion proteins that can be easily detected and/or purified, including, but not limited to “BLUESCRIPT” (a-galactosidase; Stratagene) or pGEX (glutathione S-transferase; Promega, Madison, Wis.). A suitable bacterial promoter is any nucleic acid sequence capable of binding bacterial RNA polymerase and initiating the downstream (3′) transcription of the coding sequence of the CPP gene into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tat promoter is a hybrid of the trp and lac promoter sequences. Furthermore, a bacterial promoter can include naturally occurring promoters of non-bacterial origin that have the ability to bind bacterial RNA polymerase and initiate transcription. An efficient ribosome-binding site is also desirable. The expression vector may also include a signal peptide sequence that provides for secretion of the CPP in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include drug resistance genes such as ampicillin, chloramphenicol, erythromycin, kanamycin, neomycin and tetracycline. Selectable markers also include biosynthetic genes, such as those in the histidine, tryptophan and leucine biosynthetic pathways. When large quantities of CPPs are needed, e.g., for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the CPP coding sequence may be ligated into the vector in-frame with sequences for the amino-terminal Met and the subsequent 7 residues of beta-galactosidase so that a hybrid protein is produced; PIN vectors (Van Heeke & Schuster J Biol Chem 264:5503-5509 1989)); PET vectors (Novagen, Madison Wis.); and the like. Expression vectors for bacteria include the various components set forth above, and are well known in the art. Examples include vectors for Bacillus subtilis, E. coli, Streptococcus cremoris, and Streptococcus lividans, among others. Bacterial expression vectors are transformed into bacterial host cells using techniques well known in the art, such as calcium chloride mediated transfection, electroporation, and others.

Expression in Yeast

Yeast expression systems are well known in the art, and include expression vectors for Saccharomyces cerevisiae, Candida albicans and C. maltosa, Hansenula polymorpha, Kluyveromyces fragilis and K lactis, Pichia guillermondii and P pastoris, Schizosaccharomyces pombe, and Yarrowia lipolytica. Examples of suitable promoters for use in yeast hosts include the promoters for 3-phosphoglycerate kinase (Hitzeman et al., J. Biol. Chem. 255:2073 (1980)) or other glycolytic enzymes (Hess et al., J. Adv. Enzyme Reg. 7:149 (1968); Holland, Biochemistry 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, alpha factor, the ADH2IGAPDH promoter, glucokinase alcohol oxidase, and PGH. See, for example, Ausubel, et al., 1990; Grant et al., Methods in Enzymology 153:516-544, (1987). Other yeast promoters, which are inducible have the additional advantage of transcription controlled by growth conditions, include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast selectable markers include ADE2.HIS4.LEU2.TRP1. and ALG7, which confers resistance to tunicamycin; the neomycin phosphotransferase gene, which confers resistance to G418; and the CUP1 gene, which allows yeast to grow in the presence of copper ions. Yeast expression vectors can be constructed for intracellular production or secretion of a CPP from the DNA encoding the CPP of interest. For example, a selected signal peptide and the appropriate constitutive or inducible promoter may be inserted into suitable restriction sites in the selected plasmid for direct intracellular expression of the CPP. For secretion of the CPP, DNA encoding the CPP can be cloned into the selected plasmid, together with DNA encoding the promoter, the yeast alpha-factor secretory signal/leader sequence, and linker sequences (as needed), for expression of the CPP. Yeast cells, can then be transformed with the expression plasmids described above, and cultured in an appropriate fermentation media. The protein produced by such transformed yeast can then be concentrated by precipitation with 10% trichloroacetic acid and analyzed following separation by SDS-PAGE and staining of the gels with Coomassie Blue stain. The recombinant CPP can subsequently be isolated and purified from the fermentation medium by techniques known to those of skill in the art.

Expression in Mammalian Systems

The CPP may be expressed in mammalian cells. Mammalian expression systems are known in the art, and include retroviral vector mediated expression systems. Mammalian host cells may be transformed with any of a number of different viral-based expression systems, such as adenovirus, where the coding region can be ligated into an adenovirus transcription/translation complex consisting of the late promoter and tripartite leader sequence. Insertion in a nonessential E1 or E3 region of the viral genome results in a viable virus capable of expression of the polypeptide of interest in infected host cells. A preferred expression vector system is a retroviral vector system such as is generally described in PCT/US97/01019 and PCT/US97/101048. Suitable mammalian expression vectors contain a mammalian promoter which is any DNA sequence capable of binding mammalian RNA polymerase and initiating the downstream (3′) transcription of a coding sequence for CPP into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems. Transcription of DNA encoding a CPP by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer, the cytomegalovirus early promoter enhancer, the polyoma enhancer on the late side of the replication origin, and adenovirus enhancers. The enhancer is preferably located at a site 5′ from the promoter. In general the transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Long term, high-yield production of recombinant proteins can be effected in a stable expression system. Expression vectors which contain viral origins of replication or endogenous expression elements and a selectable marker gene may be used for this purpose. Appropriate vectors containing selectable markers for use in mammalian cells are readily available commercially and are known to persons skilled in the art. Examples of such selectable markers include, but are not limited to herpes simplex virus thymidine kinase and adenine phosphoribosyltransferase for use in tk- or hprt-cells, respectively. The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

CPPs can be purified from culture supernatants of mammalian cells transiently transfected or stably transformed by an expression vector carrying a CPP-encoding sequence. Preferably, CPP is purified from culture supernatants of COS 7 cells transiently transfected by the pcD expression vector. Transfection of COS 7 cells with pcD proceeds as follows: One day prior to transfection, approximately 10⁶ COS 7 monkey cells are seeded onto individual 100 mm plates in Dulbecco's modified Eagle medium (DME) containing 10% fetal calf serum and 2 mM glutamine. To perform the transfection, the medium is aspirated from each plate and replaced with 4 ml of DME containing 50 mM Tris.HCl pH 7.4, 400 mg/ml DEAE-Dextran and 50 μg of plasmid DNA. The plates are incubated for four hours at 37° C., then the DNA-containing medium is removed, and the plates are washed twice with 5 ml of serum-free DME. DME is added back to the plates which are then incubated for an additional 3 hrs at 37° C. The plates are washed once with DME, after which DME containing 4% fetal calf serum, 2 mM glutamine, penicillin (100 μL) and streptomycin (100 μg/L) at standard concentrations is added. The cells are then incubated for 72 hrs at 37° C., after which the growth medium is collected for purification of CPP. Plasmid DNA for the transfections is obtained by growing pcD(SRα), or like expression vector, containing the CPP-encoding cDNA insert in E. coli MC1061 (described by Casadaban and Cohen, J. Mol. Biol., Vol. 138, pgs. 179-207 (1980)), or like organism. The plasmid DNA is isolated from the cultures by standard techniques, e.g. Sambrook et al., Molecular Cloning: A Laboratory Manual, Second Edition (Cold Spring Harbor Laboratory, New York 1989) or Ausubel et al (1990, cited above).

Expression in Insect Cells

CPPs may also be produced in insect cells. Expression vectors for the transformation of insect cells, and in particular, baculovirus-based expression vectors, are well known in the art. In one such system, the CPP-encoding DNA is fused upstream of an epitope tag contained within a baculovirus expression vector. Autographa californica nuclear polyhedrosis virus (AcNPV) is used as a vector to express foreign genes in Spodoptera frugiperda Sf9 cells or in Trichoplusia larvae. The CPP-encoding sequence is cloned into a nonessential region of the virus, such as the polyhedrin gene, and placed under control of the polyhedrin promoter. Successful insertion of a CPP-encoding sequence will render the polyhedrin gene inactive and produce recombinant virus lacking coat protein coat. The recombinant viruses are then used to infect S. frugiperda cells or Trichoplusia larvae in which the CPP is expressed (Smith et al., J. Wol. 46:584 (1994); Engelhard E K. et al., Proc. Nat. Acad. Sci. 91:3224-3227 (1994)). Suitable epitope tags for fusion to the CPP-encoding DNA include poly-his tags and immunoglobulin tags (like Fc regions of IgG). A variety of plasmids may be employed, including commercially available plasmids such as pVL1393 (Novagen). Briefly, the CPP-encoding DNA or the desired portion of the CPP-encoding DNA is amplified by PCR with primers complementary to the 5′ and 3′ regions. The 5′ primer may incorporate flanking restriction sites. The PCR product is then digested with the selected restriction enzymes and subcloned into an expression vector. Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL), or other methods known to those of skill in the art. Virus is produced by day 4-5 of culture in Sf9 cells at 28° C., and used for further amplifications. Procedures are performed as further described in O'Reilley et al., BACULOVIRUS EXPRESSION VECTORS: A LABORATORY MANUAL, Oxford University Press (1994). Extracts may be prepared from recombinant virus-infected Sf9 cells as described in Rupert et al., Nature 362:175-179 (1993). Alternatively, expressed epitope-tagged CPP can be purified by affinity chromatography, or for example, purification of an IgG tagged (or Fc tagged) CPP can be performed using chromatography techniques, including Protein A or protein G column chromatography.

Evaluation of Gene Expression

Gene expression may be evaluated in a sample directly, for example, by standard techniques known to those of skill in the art, e.g., Northern blotting to determine the transcription of RNA, dot blotting (DNA or RNA), or in situ hybridization, using an appropriately labeled probe, based on the sequences provided herein. Alternatively, antibodies may be used in assays for detection of polypeptides, nucleic acids, such as specific duplexes, including DNA duplexes, RNA duplexes, and DNA-RNA hybrid duplexes or DNA-protein duplexes. Such antibodies may be labeled and the assay carried out where the duplex is bound to a surface, so that upon the formation of duplex on the surface, the presence of antibody bound to the duplex can be detected. Gene expression, alternatively, may be measured by immunohistochemical staining of cells or tissue sections and assay of cell culture or body fluids, to directly evaluate the expression of a CPP polypeptide or polynucleotide. Antibodies useful for such immunological assays may be either monoclonal or polyclonal, and may be prepared against a native sequence CPP. Protein levels may also be detected by mass spectrometry. A further method of protein detection is with protein chips.

Purification of Expressed Protein

Expressed CPP may be purified or isolated after expression, using any of a variety of methods known to those skilled in the art. The appropriate technique will vary depending upon what other components are present in the sample. Contaminant components that are removed by isolation or purification are materials that would typically interfere with diagnostic or therapeutic uses for the polypeptide, and may include enzymes, hormones, and other solutes. The purification step(s) selected will depend, for example, on the nature of the production process used and the particular CPP produced. As CPPs are secreted, they may be recovered from culture medium. Alternatively, the CPP may be recovered from host cell lysates. If membrane-bound, it can be released from the membrane using a suitable detergent solution (e.g. Triton-X 100) or by enzymatic cleavage. Alternatively, cells employed in expression of CPP can be disrupted by various physical or chemical means, such as freeze-thaw cycling, sonication, mechanical disruption, or by use of cell lysing agents. Exemplary purification methods include, but are not limited to, ion-exchange column chromatography; chromatography using silica gel or a cation-exchange resin such as DEAE; gel filtration using, for example, Sephadex G-75; protein A Sepharose columns to remove contaminants such as IgG; chromatography using metal chelating columns to bind epitope-tagged forms of the CPP; ethanol precipitation; reverse phase HPLC; chromatofocusing; SDS-PAGE; and ammonium sulfate precipitation. Ordinarily, an isolated CPP will be prepared by at least one purification step. For example, the CPP may be purified using a standard anti-CPP antibody column. Ultrafiltration and dialysis techniques, in conjunction with protein concentration, are also useful (see, for example, Scopes, R., PROTEIN PURIFICATION, Springer-Verlag, New York N.Y., 1982). The degree of purification necessary will vary depending on the use of the CPP. In some instances no purification will be necessary. Once expressed and purified as needed, the CPPs and nucleic acids of the present invention are useful in a number of applications, as detailed herein.

Transgenic Animals

The host cells of the invention can also be used to produce nonhuman transgenic animals. For example, in one embodiment, a host cell of the invention is a fertilized oocyte or an embryonic stem cell into which CPP-coding sequences have been introduced. Such host cells can then be used to create non-human transgenic animals in which exogenous CPP sequences have been introduced into their genome or homologous recombinant animals in which endogenous CPP sequences have been altered. Such animals are useful for studying the function and/or activity of a CPP or fragment thereof and for identifying and/or evaluating modulators of CPP biological activity. As used herein, a “transgenic animal” is a non-human animal, preferably a mammal, more preferably a rodent such as a rat or mouse, in which one or more of the cells of the animal include a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a CPP-encoding nucleic acid into the male pronuclei of a fertilized oocyte, e.g., by microinjection or retroviral infection, and allowing the oocyte to develop in a pseudopregnant female foster animal. The CPP cDNA sequence or a fragment thereof can be introduced as a transgene into the genome of a non-human animal. Alternatively, a nonhuman homologue of a human CPP-encoding gene, such as from mouse or rat, can be used as a transgene. Intronic sequences and polyadenylation signals can also be included in the transgene to increase the efficiency of expression of the transgene. A tissue-specific regulatory sequence(s) can be operably linked to a CPP transgene to direct expression of a CPP to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986, the disclosure of which is incorporated herein by reference in its entirety). Similar methods are used for production of other transgenic animals. A transgenic founder animal can be identified based upon the presence of a CPP transgene in its genome and/or expression of CPP mRNA in tissues or cells of the animals. A transgenic founder animal can then be used to breed additional animals carrying the transgene. Moreover, transgenic animals carrying a transgene encoding a CPP can further be bred to other transgenic animals carrying other transgenes.

To create an animal in which a desired nucleic acid has been introduced into the genome via homologous recombination, a vector is prepared which contains at least a portion of a CPP-encoding sequence into which a deletion, addition or substitution has been introduced to thereby alter, e.g., functionally disrupt, the CPP-encoding sequence. The CPP-encoding sequence can be a human gene, but more preferably, is a non-human homologue of a human CPP-encoding sequence (e.g., a cDNA isolated by stringent hybridization with a nucleotide sequence coding for a CPP). For example, a mouse CPP-encoding sequence can be used to construct a homologous recombination vector suitable for altering an endogenous gene in the mouse genome. In a preferred embodiment, the vector is designed such that, upon homologous recombination, the endogenous CPP-encoding sequence is functionally disrupted (i.e., no longer encodes a functional protein; also referred to as a “knock out” vector). Alternatively, the vector can be designed such that, upon homologous recombination, the endogenous CPP-encoding sequence is mutated or otherwise altered but still encodes functional protein (e.g., the upstream regulatory region can be altered to thereby alter the expression of the endogenous CPP-encoding sequence). In the homologous recombination vector, the altered portion of the CPP-encoding sequence is flanked at its 5′ and 3′ ends by additional nucleic acid sequence of the CPP gene to allow for homologous recombination to occur between the exogenous sequence carried by the vector and an endogenous gene in an embryonic stem cell. The additional flanking nucleic acid sequence is of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503, the disclosure of which is incorporated herein by reference in its entirety, for a description of homologous recombination vectors). The vector is introduced into an embryonic stem cell line (e.g., by electroporation) and cells in which the introduced CPP-encoding sequence has homologously recombined with the endogenous gene are selected (see e.g., Li, E. et al. (1992) Cell 69:915, the disclosure of which is incorporated herein by reference in its entirety). The selected cells are then injected into a blastocyst of an animal (e.g., a mouse) to form aggregation chimeras (see e.g., Bradley, A. in Teratocarcinomas and Embryonic Stem Cells. A Practical Approach, E. J. Robertson, ed. (IRL, Oxford, 1987) pp. 113-152, the disclosure of which is incorporated herein by reference in its entirety). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al., the disclosures of which are incorporated herein by reference in their entireties.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236, the disclosure of which is incorporated herein by reference in its entirety. Another example of a recombinase system is the FLP recombinase system of Saccharomyces cerevisiae (O'Gorman et al. (1991) Science 251:1351-1355, the disclosure of which is incorporated herein by reference in its entirety). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Assessing CPP Activity

It will be appreciated that the invention further provides methods of testing the activity of or obtaining functional fragments and variants of CPPs and CPP sequences. Such methods involve providing a variant or modified CPP-encoding nucleic acid and assessing whether the encoded polypeptide displays a CPP biological activity. Encompassed is thus a method of assessing the function of a CPP comprising: (a) providing a CPP, or a biologically active fragment or homologue thereof; and (b) testing said CPP, or a biologically active fragment or homologue thereof for a CPP biological activity under conditions suitable for CPP activity. Cell free, cell-based and in vivo assays may be used to test CPP activity. For example, said assay may comprise expressing a CPP nucleic acid in a host cell, and observing CPP activity in said cell and other affected cells. In another example, a CPP, or a biologically active fragment or homologue thereof is contacted with a cell, and a CPP biological activity is observed.

CPP biological activities include: (1) indicating that an individual has or will have a cardiovascular disorder; (2) circulating through the bloodstream of individuals with a cardiovascular disorder, (3) antigenicity, or the ability to bind an anti-CPP specific antibody; (4) immunogenicity, or the ability to generate an anti-CPP specific antibody; (5) forming intermolecular amino acid side chain interactions such as hydrogen, amide, or preferably disulfide links; (6) interaction with a CPP target molecule, or in particular, an endothelial cell surface; and (7) undergoing posttranslational processing, in particular, specific protealysis or glycosylation.

CPP biological activity can be assayed by any suitable method known in the art. Antigenicity and immunogenicity may be detected, for example, as described in the sections titled “Anti CPP antibodies” and “Uses of CPP antibodies”. Circulation in blood plasma may be detected as described in “Diagnostic and Prognostic Uses”. Determining the ability of the CPP to bind to or interact with a CPP target molecule can be accomplished by a method for directly or indirectly determining binding, as is common to the art. Such methods are further described in the section titled “Drug screening assays.”

Determining the ability of the CPP to bind to or interact with a CPP target molecule can be accomplished by a method for directly or indirectly determining binding, as is common to the art. Such methods can be cell-based (e.g., such that binding to a membrane-bound CPP is detected) or cell free. Interaction of a test compound with a CPP can be detected, for example, by coupling the CPP or biologically active portion thereof with a label group such that binding of the CPP or biologically active portion thereof to its cognate target molecule can be determined by detecting the labeled CPP or biologically active portion thereof in a complex. For example, the extent of complex formation may be measured by immunoprecipitating the complex or by performing gel electrophoresis. Determining the ability of the CPP to bind to a CPP target molecule may also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct Biol. 5:699-705, the disclosures of which are incorporated herein by reference in their entireties. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

Cardiovascular disorders may be diagnosed by any method determined appropriate for an individual by one of skill in the art. Further examples of symptoms and diagnostics may be found in the Background section, and are best determined appropriately by one of skill in the art based on the particular profile of a patient.

Intramolecular interactions may be detected by sequence-based structural predictions. Such predictions are generally based on X-ray crystallography or NMR structural data for a polypeptide with similar sequence. Detection of intramolecular interactions may also be accomplished using SDS-PAGE. For the example of disulfide bonds, links formed between different portions of a given protein result in a more compacted protein, and thus, a reduced apparent molecular weight. Disulfide bonds may be disrupted by a reducing agent, for example, dithiothreitol (DTT). A protein sample that has been treated with a reducing agent may thus be compared to an untreated control by SDS-PAGE to detect a change in apparent molecular weight. Such methods are common to the art.

Association with an endothelial cell surface may be detected, for example, by immunodetection techniques as is common in the art. HUVEC cells or in situ vesicular membranes may be used for detection assays. Christian, et al. (JBC, 276:48588-48595 (2001), disclosure of which is incorporated herein by reference in its entirety) describe an exemplary immunohistochemical method of detection by comparing localization of the protein of interest to that of VE-Cadherin.

Glycosylation state may be determined, for example, as described in EP Patent 0693559B1. Other common methods include comparison of observed and expected molecular weights for an arginine-containing peptide sequence, wherein a higher than expected molecular weight indicates that the peptide is glycolsylated. Specific proteolysis may also be detected by comparing the molecular weight of a sample peptide to that of a peptide of known molecular weight. Molecular weights are easily compared according to any method common to the art such as SDS-PAGE, gel chromatography, or mass spectrometry. Preferably, the molecular weight of a test peptide is obtained by mass spectrometry and compared to a database comprising molecular weights of peptides with posttranslational modifications. Exemplary databases include Genpept, SWISSPROT, EMBL, and the Protein Sequence Database. Such techniques are detailed further herein.

Anti-CPP Antibodies

The present invention provides antibodies and binding compositions specific for CPPs. Such antibodies and binding compositions include polyclonal antibodies, monoclonal antibodies, Fab and single chain Fv fragments thereof, bispecific antibodies, heteroconjugates, and humanized antibodies. Such antibodies and binding compositions may be produced in a variety of ways, including hybridoma cultures, recombinant expression in bacteria or mammalian cell cultures, and recombinant expression in transgenic animals. There is abundant guidance in the literature for selecting a particular production methodology, e.g. Chadd and Chamow, Curr. Opin. Biotechnol., 12: 188-194 (2001).

The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments are highly selective for in vivo tumors, show good tumor penetration and low immunogenicity, and are cleared rapidly from the blood, e.g. Freyre et al, J. Biotechnol., 76: 157-163 (2000). Thus, such molecules are desirable for radioimmunodetection and in situ radiotherapy. Whenever pharmacokinetic activity in the form of increased half-life is required for therapeutic purposes, then full-length antibodies are preferable. For example, the immunoglobulin G (IgG) molecule may be one of four subclasses: γ1, γ2, γ3, or γ4. If a full-length antibody with effector function is required, then IgG subclasses γ1 or γ3 are preferred, and IgG subclass γ1 is most preferred. The γ1 and γ3 subclasses exhibit potent effector function, complement activation, and promote antibody-dependent cell-mediated cytotoxicity through interaction with specific Fc receptors, e.g. Raju et al, Glycobiology, 10: 477-486 (2000); Lund et al, J. Immunol., 147: 2657-2662 (1991).

Polyclonal Antibodies

The anti-CPP antibodies of the present invention may be polyclonal antibodies. Such polyclonal antibodies can be produced in a mammal, for example, following one or more injections of an immunizing agent, and preferably, an adjuvant. Typically, the immunizing agent and/or adjuvant will be injected into the mammal by a series of subcutaneous or intraperitoneal injections. The immunizing agent may include CPPs or a fusion protein thereof. It may be useful to conjugate the antigen to a protein known to be immunogenic in the mammal being immunized. Examples of such immunogenic proteins include, but are not limited to, keyhole limpet hemocyanin (KLH), methylated bovine serum albumin (mBSA), bovine serum albumin (BSA), Hepatitis B surface antigen, serum albumin, bovine thyroglobulin, and soybean trypsin inhibitor. Adjuvants include, for example, Freund's complete adjuvant and MPL-TDM adjuvant (monophosphoryl Lipid A, synthetic trehalose dicoryno-mycolate). The immunization protocol may be determined by one skilled in the art based on standard protocols or by routine experimentation.

Alternatively, a crude protein preparation which has been enriched for a CPP or a portion thereof can be used to generate antibodies. Such proteins, fragments or preparations are introduced into the non-human mammal in the presence of an appropriate adjuvant. If the serum contains polyclonal antibodies to undesired epitopes, the polyclonal antibodies are purified by immunoaffinity chromatography.

Effective polyclonal antibody production is affected by many factors related both to the antigen and the host species. Also, host animals vary in response to site of inoculations and dose, with both inadequate and excessive doses of antigen resulting in low titer antisera. Small doses (ng level) of antigen administered at multiple intradermal sites appear to be most reliable. Techniques for producing and processing polyclonal antisera are known in the art, see for example, Mayer and Walker (1987), the disclosure of which is incorporated herein by reference in its entirety. An effective immunization protocol for rabbits can be found in Vaitukaitis, J. et al. J. Clin. Endocrinol. Metab. 33:988-991 (1971), the disclosure of which is incorporated by reference in its entirety. Booster injections can be given at regular intervals, and antiserum harvested when antibody titer thereof, as determined semi-quantitatively, for example, by double immunodiffusion in agar against known concentrations of the antigen, begins to fall. See, for example, Ouchterlony, O. et al., Chap. 19 in: Handbook of Experimental Immunology D. Wier (ed) Blackwell (1973). Plateau concentration of antibody is usually in the range of 0.1 to 0.2 mg/ml of serum. Affinity of the antisera for the antigen is determined by preparing competitive binding curves, as described, for example, by Fisher, D., Chap. 42 in: Manual of Clinical Immunology, 2d Ed. (Rose and Friedman, Eds.) Amer. Soc. For Microbiol., Washington, D.C. (1980).

Monoclonal Antibodies

Alternatively, the anti-CPP antibodies may be monoclonal antibodies. Monoclonal antibodies may be produced by hybridomas, wherein a mouse, hamster, or other appropriate host animal, is immunized with an immunizing agent to elicit lymphocytes that produce or are capable of producing antibodies that will specifically bind to the immunizing agent, e.g. Kohler and Milstein, Nature 256:495 (1975). The immunizing agent will typically include the CPP or a fusion protein thereof and optionally a carrier. Alternatively, the lymphocytes may be immunized in vitro. Generally, spleen cells or lymph node cells are used if non-human mammalian sources are desired, or peripheral blood lymphocytes (“PBLs”) are used if cells of human origin are desired. The lymphocytes are fused with an immortalized cell line using a suitable fusing agent, such as polyethylene glycol, to produce a hybridoma cell, e.g. Goding, MONOCLONAL ANTIBODIES: PRINCIPLES AND PRACTICE, Academic Press, pp. 59-103 (1986); Liddell and Cryer, A Practical Guide to Monoclonal Antibodies (John Wiley & Sons, New York, 1991); Malik and Lillenoj, Editors, Antibody Techniques (Academic Press, New York, 1994). In general, immortalized cell lines are transformed mammalian cells, for example, myeloma cells of rat, mouse, bovine or human origin. The hybridoma cells are cultured in a suitable culture medium that preferably contains one or more substances that inhibit the growth or survival of unfused, immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT), substances which prevent the growth of HGPRT-deficient cells. Preferred immortalized cell lines are those that fuse efficiently, support stable high level production of antibody, and are sensitive to a medium such as HAT medium. More preferred immortalized cell lines are murine or human myeloma lines, which can be obtained, for example, from the American Type Culture Collection (ATCC), Rockville, Md. Human myeloma and mouse-human heteromyeloma cell lines also have been described for the production of human monoclonal antibodies, e.g. Kozbor, J. Immunol. 133:3001 (1984); Brodeur et al., Monoclonal Antibody Production Techniques and Applications, Marcel Dekker, Inc., New York, pp. 51-63 (1987).

The culture medium (supernatant) in which the hybridoma cells are cultured can be assayed for the presence of monoclonal antibodies directed against a CPP. Preferably, the binding specificity of monoclonal antibodies present in the hybridoma supernatant is determined by immunoprecipitation or by an in vitro binding assay, such as radio-immunoassay (RIA) or Enzyme-Linked Immuno Sorbent Assay (ELISA). Appropriate techniques and assays are known in the art. The binding affinity of the monoclonal antibody can, for example, be determined by the Scatchard analysis of Munson and Pollard, Anal. Biochem. 107:220 (1980). After the desired antibody-producing hybridoma cells are identified, the cells may be cloned by limiting dilution procedures and grown by standard methods (Goding, 1986, supra). Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by selected clones may be isolated or purified from the culture medium or ascites fluid by immunoglobulin purification procedures routinely used by those of skill in the art such as, for example, protein A-Sepharose, hydroxyl-apatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be isolated from the CPP-specific hybridoma cells and sequenced, e.g., by using oligonucleotide probes that are capable of binding specifically to genes encoding the heavy and light chains of murine antibodies. Once isolated, the DNA may be inserted into an expression vector, which is then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for the murine heavy and light chain constant domains for the homologous human sequences (Morrison et al., Proc. Nat. Acad. Sci. 81:6851-6855 (1984); Neuberger et al., Nature 312:604-608 (1984); Takeda et al., Nature 314:452-454 (1985)), or by covalently joining to the immunoglobulin coding sequence all or part of the coding sequence for a non-immunoglobulin polypeptide. The non-immunoglobulin polypeptide can be substituted for the constant domains of an antibody of the invention, or can be substituted for the variable domains of one antigen-combining site of an antibody of the invention to create a chimeric bivalent antibody. The antibodies may also be monovalent antibodies. Methods for preparing monovalent antibodies are well known in the art. For example, in vitro methods are suitable for preparing monovalent antibodies. Digestion of antibodies to produce fragments thereof, particularly, Fab fragments, can be accomplished using routine techniques known in the art.

Antibodies and antibody fragments characteristic of hybridomas of the invention can also be produced by recombinant means by extracting messenger RNA, constructing a cDNA library, and selecting clones which encode segments of the antibody molecule. The following are exemplary references disclosing recombinant techniques for producing antibodies: Wall et al., Nucleic Acids Research, Vol. 5, pgs. 3113-3128 (1978); Zakut et al., Nucleic Acids Research, Vol. 8, pgs. 3591-3601 (1980); Cabilly et al., Proc. Natl. Acad. Sci., Vol. 81, pgs. 3273-3277 (1984); Boss et al., Nucleic Acids Research, Vol. 12, pgs. 3791-3806 (1984); Amster et al., Nucleic Acids Research, Vol. 8, pgs. 2055-2065 (1980); Moore et al., U.S. Pat. No. 4,642,334; Skerra et al, Science, Vol. 240, pgs. 1038-1041 (1988); Huse et al, Science, Vol. 246, pgs. 1275-1281 (1989); and U.S. Pat. Nos. 6,054,297; 5,530,101; 4,816,567; 5,750,105; and 5,648,237; which patents are incorporated by reference. In particular, such techniques can be used to produce interspecific monoclonal antibodies, wherein the binding region of one species is combined with non-binding region of the antibody of another species to reduce immunogenicity, e.g. Liu et al., Proc. Natl. Acad. Sci., Vol. 84, pgs. 3439-3443 (1987), and U.S. Pat. Nos. 6,054,297 and 5,530,101. Preferably, recombinantly produced Fab and Fv fragments are expressed in bacterial host systems. Preferably, full-length antibodies are produced by mammalian cell culture techniques. More preferably, full-length antibodies are expressed in Chinese Hamster Ovary (CHO) cells or NSO cells.

Both polyclonal and monoclonal antibodies can be screened by ELISA. As in other solid phase immunoassays, the test is based on the tendency of macromolecules to adsorb nonspecifically to plastic. The irreversibility of this reaction, without loss of immunological activity, allows the formation of antigen-antibody complexes with a simple separation of such complexes from unbound material. To titrate anti-peptide serum, peptide conjugated to a carrier different from that used in immunization is adsorbed to the wells of a 96-well microtiter plate. The adsorbed antigen is then allowed to react in the wells with dilutions of anti-peptide serum. Unbound antibody is washed away, and the remaining antigen-antibody complexes are allowed to react with an antibody specific for the IgG of the immunized animal. This second antibody is conjugated to an enzyme such as alkaline phosphatase. A visible colored reaction produced when the enzyme substrate is added indicates which wells have bound antipeptide antibodies. The use of spectrophotometer readings allows better quantification of the amount of peptide-specific antibody bound. High-titer antisera yield a linear titration curve between 10⁻³ and 10⁻⁵ dilutions.

CPP Peptide Carriers

The invention includes immunogens derived from CPPs, and immunogens comprising conjugates between carriers and peptides of the invention. The term immunogen as used herein refers to a substance which is capable of causing an immune response. The term carrier as used herein refers to any substance which when chemically conjugated to a peptide of the invention permits a host organism immunized with the resulting conjugate to generate antibodies specific for the conjugated peptide. Carriers include red blood cells, bacteriophages, proteins, or synthetic particles such as agarose beads. Preferably, carriers are proteins, such as serum albumin, gamma-globulin, keyhole limpet hemocyanin (KLH), thyroglobulin, ovalbumin, or fibrinogen.

The general technique of linking synthetic peptides to a carrier is described in several references, e.g. Walter and Doolittle, “Antibodies Against Synthetic Peptides,” in Setlow et al., eds., Genetic Engineering, Vol. 5, pgs. 61-91 (Plenum Press, N.Y., 1983); Green et al. Cell, Vol. 28, pgs. 477-487 (1982); Lerner et al., Proc. Natl. Acad. Sci., Vol. 78, pgs. 3403-3407 (1981); Shimizu et al., U.S. Pat. No. 4,474,754; and Ganfield et al., U.S. Pat. No. 4,311,639. Accordingly, these references are incorporated by reference. Also, techniques employed to link haptens to carriers are essentially the same as the above-referenced techniques, e.g. chapter 20 in Tijssen, Practice and Theory of Enzyme Immunoassays (Elsevier, New York, 1985). The four most commonly used schemes for attaching a peptide to a carrier are (1) glutaraldehyde for amino coupling, e.g. as disclosed by Kagan and Glick, in Jaffe and Behrman, eds. Methods of Hormone Radioimmunoassay, pgs. 328-329 (Academic Press, N.Y., 1979), and Walter et al. Proc. Natl. Acad. Sci., Vol. 77, pgs. 5197-5200 (1980); (2) water-soluble carbodiimides for carboxyl to amino coupling, e.g. as disclosed by Hoare et al., J. Biol. Chem., Vol. 242, pgs. 2447-2453 (1967); (3) bis-diazobenzidine (BDB) for tyrosine to tyrosine sidechain coupling, e.g. as disclosed by Bassiri et al., pgs. 46-47, in Jaffe and Behrman, eds. (cited above), and Walter et al. (cited above); and (4) maleimidobenzoyl-N-hydroxysuccinimide ester (MBS) for coupling cysteine (or other sulfhydryls) to amino groups, e.g. as disclosed by Kitagawa et al., J. Biochem. Tokyo), Vol. 79, pgs. 233-239 (1976), and Lerner et al. (cited above). A general rule for selecting an appropriate method for coupling a given peptide to a protein carrier can be stated as follows: the group involved in attachment should occur only once in the sequence, preferably at the appropriate end of the segment. For example, BDB should not be used if a tyrosine residue occurs in the main part of a sequence chosen for its potentially antigenic character. Similarly, centrally located lysines rule out the glutaraldehyde method, and the occurrences of aspartic and glutamic acids frequently exclude the carbodiimide approach. On the other hand, suitable residues can be positioned at either end of chosen sequence segment as attachment sites, whether or not they occur in the “native” protein sequence. Internal segments, unlike the amino and carboxy termini, will differ significantly at the “unattached end” from the same sequence as it is found in the native protein where the polypeptide backbone is continuous. The problem can be remedied, to a degree, by acetylating the α-amino group and then attaching the peptide by way of its carboxy terminus. The coupling efficiency to the carrier protein is conveniently measured by using a radioactively labeled peptide, prepared either by using a radioactive amino acid for one step of the synthesis or by labeling the completed peptide by the iodination of a tyrosine residue. The presence of tyrosine in the peptide also allows one to set up a sensitive radioimmune assay, if desirable. Therefore, tyrosine can be introduced as a terminal residue if it is not part of the peptide sequence defined by the native polypeptide.

Preferred carriers are proteins, and preferred protein carriers include bovine serum albumin, myoglobulin, ovalbumin (OVA), keyhole limpet hemocyanin (KLH), or the like. Peptides can be linked to KLH through cysteines by MBS as disclosed by Liu et al., Biochemistry, Vol. 18, pgs. 690-697 (1979). The peptides are dissolved in phosphate-buffered saline (pH 7.5), 0.1 M sodium borate buffer (pH 9.0) or 1.0 M sodium acetate buffer (pH 4.0). The pH for the dissolution of the peptide is chosen to optimize peptide solubility. The content of free cysteine for soluble peptides is determined by Ellman's method, Ellman, Arch. Biochem. Biophys., Vol. 82, pg. 7077 (1959). For each peptide, 4 mg KLH in 0.25 ml of 10 mM sodium phosphate buffer (pH 7.2) is reacted with 0.7 mg MBS (dissolved in dimethyl formamide) and stirred for 30 min at room temperature. The MBS is added dropwise to ensure that the local concentration of formamide is not too high, as KLH is insoluble in >30% formamide. The reaction product, KLH-MBS, is then passed through Sephadex G-25 equilibrated with 50 mM sodium phosphate buffer (pH 6.0) to remove free MBS, KLH recovery from peak fractions of the column eluate (monitored by OD280) is estimated to be approximately 80%. KLH-MBS is then reacted with 5 mg peptide dissolved in 1 ml of the chosen buffer. The pH is adjusted to 7-7.5 and the reaction is stirred for 3 hr at room temperature. Coupling efficiency is monitored with radioactive peptide by dialysis of a sample of the conjugate against phosphate-buffered saline, and may range from 8% to 60%. Once the peptide-carrier conjugate is available, polyclonal or monoclonal antibodies are produced by standard techniques, e.g. as disclosed by Campbell, Monoclonal Antibody Technology (Elsevier, New York, 1984); Hurrell, ed. Monoclonal Hybridoma Antibodies: Techniques and Applications (CRC Press, Boca Raton, Fla., 1982); Schreier et al. Hybridoma Techniques (Cold Spring Harbor Laboratory, New York, 1980); U.S. Pat. No. 4,562,003; or the like. In particular, U.S. Pat. No. 4,562,003 is incorporated by reference.

Humanized Antibodies

The anti-CPP antibodies of the invention may further comprise humanized antibodies or human antibodies. The term “humanized antibody” refers to humanized forms of non-human (e.g., murine) antibodies that are chimeric antibodies, immunoglobulin chains or fragments thereof (such as Fv, Fab, Fab′, F(ab′), or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986) and Presta, Curr. Op. Struct. Biol. 2:593-596 (1992)). Methods for humanizing non-human antibodies are well known in the art. Generally, a humanized antibody has one or more amino acids introduced into it from a source which is non-human in order to more closely resemble a human antibody, while still retaining the original binding activity of the antibody. Methods for humanization of antibodies are further detailed in Jones et al., Nature 321:522-525 (1986); Riechmann et al., Nature 332:323-327 (1988); and Verhoeyen et al., Science 239:1534-1536 (1988). Such “humanized” antibodies are chimeric antibodies in that substantially less than an intact human variable domain has been substituted by the corresponding sequence from a non-human species.

Heteroconjugate Antibodies

Heteroconjugate antibodies which comprise two covalently joined antibodies, are also within the scope of the present invention. Heteroconjugate antibodies may be prepared in vitro using known methods in synthetic protein chemistry, including those involving crosslinking agents. For example, immunotoxins may be prepared using a disulfide exchange reaction or by forming a thioether bond.

Bispecific Antibodies

Bispecific antibodies have binding specificities for at least two different antigens. Such antibodies are monoclonal, and preferably human or humanized. One of the binding specificities of a bispecific antibody of the present invention is for a CPP, and the other one is preferably for a cell-surface protein or receptor or receptor subunit. Methods for making bispecific antibodies are known in the art, and in general, the recombinant production of bispecific antibodies is based on the co-expression of two immunoglobulin heavy-chain/light-chain pairs in hybridoma cells, where the two heavy chains have different specificities, e.g. Milstein and Cuello, Nature 305:537-539 (1983). Given that the random assortment of immunoglobulin heavy and light chains results in production of potentially ten different antibody molecules by the hybridomas, purification of the correct molecule usually requires some sort of affinity purification, e.g. affinity chromatography.

Uses of CPP Antibodies

CPP antibodies are preferably specific for the CPPs of the invention and, as such, do not bind peptides derived from other proteins with high affinity. As used herein, the term “heavy chain variable region” means a polypeptide (1) which is from 110 to 125 amino acids in length, and (2) whose amino acid sequence corresponds to that of a heavy chain of an antibody of the invention, starting from the heavy chain's N-terminal amino acid. Likewise, the term “light chain variable region” means a polypeptide (1) which is from 95 to 115 amino acids in length, and (2) whose amino acid sequence corresponds to that of a light chain of an antibody of the invention, starting from the light chain's N-terminal amino acid. As used herein the term “monoclonal antibody” refers to homogeneous populations of immunoglobulins which are capable of specifically binding to CPPs.

CPP antibodies may be used as functional modulators, preferably as antagonists. Preferably, antibody modulators of the invention are derived from monoclonal antibodies specific for CPPs. Monoclonal antibodies capable of blocking, or neutralizing, CPPs are selected by their ability to inhibit a CPP biological activity.

The use of antibody fragments is also well known, e.g. Fab fragments: Tijssen, Practice and Theory of Enzyme Immunoassays (Elsevier, Amsterdam, 1985); and Fv fragments: Hochman et al. Biochemistry, Vol. 12, pgs. 1130-1135 (1973), Sharon et al., Biochemistry, Vol. 15, pgs. 1591-1594 (1976) and Ehrlich et al., U.S. Pat. No. 4,355,023; and antibody half molecules: Auditore-Hargreaves, U.S. Pat. No. 4,470,925.

Preferably, monoclonal antibodies, Fv fragments, Fab fragments, or other binding compositions derived from monoclonal antibodies of the invention have a high affinity to CPPs. The affinity of monoclonal antibodies and related molecules to CPPs may be measured by conventional techniques including plasmon resonance, ELISA, or equilibrium dialysis. Affinity measurement by plasmon resonance techniques may be carried out, for example, using a BIAcore 2000 instrument (Biacore AB, Uppsala, Sweden) in accordance with the manufacturer's recommended protocol. Preferably, affinity is measured by ELISA, as described in U.S. Pat. No. 6,235,883, for example. Preferably, the dissociation constant between CPPs and monoclonal antibodies of the invention is less than 10⁻⁵ molar. More preferably, such dissociation constant is less than 10⁻⁸ molar; still more preferably, such dissociation constant is less than 10⁻⁹ molar; and most preferably, such dissociation constant is in the range of 10⁻⁹ to 10⁻¹¹ molar.

In addition, the antibodies of the present invention are useful for detecting CPPs. Such detection methods are advantageously applied to diagnosis of cardiovascular disorders, in particular, coronary artery disease. The antibodies of the invention may be used in most assays involving antigen-antibody reactions. The assays may be homogeneous or heterogeneous. In a homogeneous assay approach, the sample can be a biological sample or fluid such as serum, urine, whole blood, lymphatic fluid, plasma, saliva, cells, tissue, and material secreted by cells or tissues cultured in vitro. The sample can be pretreated if necessary to remove unwanted materials. The immunological reaction usually involves the specific antibody, a labeled analyte, and the sample suspected of containing the antigen. The signal arising from the label is modified, directly or indirectly, upon the binding of the antibody to the labeled analyte. Both immunological reaction and detection of the extent thereof are carried out in a homogeneous solution. Immunochemical labels which may be employed include free radicals, fluorescent dyes, enzymes, bacteriophages, coenzymes, and so forth.

In a heterogeneous assay approach, the reagents are usually the sample, the specific antibody, and means for producing a detectable signal. The specimen is generally placed on a support, such as a plate or a slide, and contacted with the antibody in a liquid phase. The support is then separated from the liquid phase and either the support phase or the liquid phase is examined for a detectable signal employing means for producing such signal or signal producing system. The signal is related to the presence of the antigen in the sample. Means for producing a detectable signal includes the use of radioactive labels, fluorescent compounds, enzymes, and so forth. Exemplary heterogeneous immunoassays are the radioimmunoassay, immunofluorescence methods, enzyme-linked immunoassays, and the like.

For a more detailed discussion of the above immunoassay techniques, see “Enzyme-Immunoassay,” by Edward T. Maggio, CRC Press, Inc., Boca Raton, Fla., 1980. See also, for example, U.S. Pat. Nos. 3,690,834; 3,791,932; 3,817,837; 3,850,578; 3,853,987; 3,867,517; 3,901,654; 3,935,074; 3,984,533; 3,966,345; and 4,098,876, which listing is not intended to be exhaustive. Methods for conjugating labels to antibodies and antibody fragments are well known in the art. Such methods may be found in U.S. Pat. Nos. 4,220,450; 4,235,869; 3,935,974; and 3,966,345. Another example of a technique in which the antibodies of the invention may be employed is immunoperoxidase labeling. (Sternberger, Immunocytochemistry (1979) pp. 104-169). Alternatively, the antibodies may be bound to a radioactive material or to a drug to form a radiopharmaceutical or pharmaceutical, respectively. (Carrasquillo, et al., Cancer Treatment Reports (1984) 68:317-328).

One embodiment of an assay employing an antibody of the present invention involves the use of a surface to which the monoclonal antibody of the invention is attached. The underlying structure of the surface may take different forms, have different compositions and may be a mixture of compositions or laminates or combinations thereof. The surface may assume a variety of shapes and forms and may have varied dimensions, depending on the manner of use and measurement. Illustrative surfaces may be pads, beads, discs, or strips which may be flat, concave or convex. Thickness is not critical, generally being from about 0.1 to 2 mm thick and of any convenient diameter or other dimensions. The surface typically will be supported on a rod, tube, capillary, fiber, strip, disc, plate, cuvette and will typically be porous and polyfunctional or capable of being polyfunctionalized so as to permit covalent binding of an antibody and permit bonding of other compounds which form a part of a means for producing a detectable signal. A wide variety of organic and inorganic polymers, both natural and synthetic, and combinations thereof, may be employed as the material for the solid surface. Illustrative polymers include polyethylene, polypropylene, poly(4-methylbutene), polystyrene, polymethracrylate, poly(ethylene terephthalate), rayon, nylon, poly(vinyl butyrate), silicones, polyformaldehyde, cellulose, cellulose acetate, nitrocellulose, and latex. Other surfaces include paper, glasses, ceramics, metals, metaloids, semiconductor materials, cements, silicates or the like. Also included are substrates that form gels, gelatins, lipopolysaccharides, silicates, agarose and polyacrylamides or polymers which form several aqueous phases such as dextrans, polyalkylene glycols (alkylene of 2 to 3 carbon atoms) or surfactants such as phospholipids. The binding of the antibody to the surface may be accomplished by well known techniques, commonly available in the literature (see, for example, “Immobilized Enzymes,” Ichiro Chibata, Press, New York (1978) and Cuatrecasas, J. Bio. Chem., 245: 3059 (1970)). In carrying out the assay in accordance with this aspect of the invention, the sample is mixed with aqueous medium and the medium is contacted with the surface having an antibody bound thereto. Labels may be included in the aqueous medium, either concurrently or added subsequently so as to provide a detectable signal associated with the surface. The means for producing the detectable signal can involve the incorporation of a labeled analyte or it may involve the use of a second monoclonal antibody having a label conjugated thereto. Separation and washing steps will be carried out as needed. The signal detected is related to the presence of CPP in the sample. It is within the scope of the present invention to include a calibration on the same support. A particular embodiment of an assay in accordance with the present invention, by way of illustration and not limitation, involves the use of a support such as a slide or a well of a petri dish. The technique involves fixing the sample to be analyzed on the support with an appropriate fixing material and incubating the sample on the slide with a monoclonal antibody. After washing with an appropriate buffer such as, for example, phosphate buffered saline, the support is contacted with a labeled specific binding partner for the antibody. After incubation as desired, the slide is washed a second time with an aqueous buffer and the determination is made of the binding of the labeled monoclonal antibody to the antigen. If the label is fluorescent, the slide may be covered with a fluorescent antibody mounting fluid on a cover slip and then examined with a fluorescent microscope to determine the extent of binding. On the other hand, the label can be an enzyme conjugated to the monoclonal antibody and the extent of binding can be determined by examining the slide for the presence of enzyme activity, which may be indicated by the formation of a precipitate, color, etc. A particular example of an assay utilizing the present antibodies is a double determinant ELISA assay. A support such as, e.g., a glass or vinyl plate, is coated with an antibody specific for CPP by conventional techniques. The support is contacted with the sample suspected of containing CPP, usually in aqueous medium. After an incubation period from 30 seconds to 12 hours, the support is separated from the medium, washed to remove unbound CPP with, for example, water or an aqueous buffered medium, and contacted with an antibody specific for CPP, again usually in aqueous medium. The antibody is labeled with an enzyme directly or indirectly such as, e.g., horseradish peroxidase or alkaline phosphatase. After incubation, the support is separated from the medium, and washed as above. The enzyme activity of the support or the aqueous medium is determined. This enzyme activity is related to the amount of CPP in the sample.

The invention also includes kits, e.g., diagnostic assay kits, for carrying out the methods disclosed above. In one embodiment, the kit comprises in packaged combination (a) a monoclonal antibody more specifically defined above and (b) a conjugate of a specific binding partner for the above monoclonal antibody and a label capable of producing a detectable signal. The reagents may also include ancillary agents such as buffering agents and protein stabilizing agents, e.g., polysaccharides and the like. The kit may further include, where necessary, other members of the signal producing system of which system the label is a member, agents for reducing background interference in a test, control reagents, apparatus for conducting a test, and the like. In another embodiment, the diagnostic kit comprises a conjugate of monoclonal antibody of the invention and a label capable of producing a detectable signal. Ancillary agents as mentioned above may also be present.

Further, an anti-CPP antibody (e.g., monoclonal antibody) can be used to isolate CPPs by standard techniques, such as affinity chromatography or immunoprecipitation. For example, an anti-CPP antibody can facilitate the purification of natural CPPs from cells and of recombinantly produced CPP expressed in host cells. Moreover, an anti-CPP antibody can be used to isolate CPP to aid in detection of low concentrations of CPP (e.g., in plasma, cellular lysate or cell supernatant) or in order to evaluate the abundance and pattern of expression of the CPP. Anti-CPP antibodies can be used diagnostically to monitor protein levels in tissue as part of a clinical testing procedure, e.g., to determine the efficacy of a given treatment regimen. Detection can be facilitated by coupling (i.e., physically linking) the antibody to a label group.

Protein Arrays

Detection, purification, and screening of the polypeptides of the invention may be accomplished using retentate chromatography (preferably, protein arrays or chips), as described by U.S. Pat. No. 6,225,027 and U.S. patent application 20010014461, disclosures of which are herein incorporated by reference in their entireties. Briefly, retentate chromatography describes methods in which polypeptides (and/or other sample components) are retained on an adsorbent (e.g., array or chip) and subsequently detected. Such methods involve (1) selectively adsorbing polypeptides from a sample to a substrate under a plurality of different adsorbent/eluant combinations (“selectivity conditions”) and (2) detecting the retention of adsorbed polypeptides by desorption spectrometry (e.g., by mass spectrometry). In conventional chromatographic methods, polypeptides are eluted off of the adsorbent prior to detection. The coupling of adsorption chromatography with detection by desorption spectrometry provides extraordinary sensitivity, the ability to rapidly analyze retained components with a variety of different selectivity conditions, and parallel processing of components adsorbed to different sites (i.e., “affinity sites” or “spots”) on the array under different elution conditions.

These methods are useful for: combinatorial, biochemical separation and purification of the CPPs; study of differential gene expression; detection of differences in protein levels (e.g., for diagnosis); and detection of molecular recognition events (e.g., for screening and drug discovery). Thus, this invention provides a molecular discovery and diagnostic device that is characterized by the inclusion of both parallel and multiplex polypeptide processing capabilities. Polypeptides of the invention and CPP-binding substances are preferably attached to a label group, and thus directly detected, enabling simultaneous transmission of two or more signals from the same “circuit” (i.e., addressable “chip” location) during a single unit operation.

Detection of CPPs by Mass Spectrometry

In accordance with the present invention, any instrument, method, process, etc. can be utilized to determine the identity and abundance of proteins in a sample. A preferred method of obtaining identity is by mass spectrometry, where protein molecules in a sample are ionized and then the resultant mass and charge of the protein ions are detected and determined.

To use mass spectrometry to analyze proteins, it is preferred that the protein be converted to a gas-ion phase. Various methods of protein ionization are useful, including, e.g., fast ion bombardment (FAB), plasma desorption, laser desorption, thermal desorption, preferably, electrospray ionization (ESI) and matrix-assisted laser desorption/ionization (MALDI). Many different mass analyzers are available for peptide and protein analysis, including, but not limited to, Time-of-Flight (TOF), ion trap (ITMS), Fourier transform ion cyclotron (FTMS), quadrupole ion trap, and sector (electric and/or magnetic) spectrometers. See, e.g., U.S. Pat. No. 5,572,025 for an ion trap MS. Mass analyzers can be used alone, or in combination with other mass analyzers in tandem mass spectrometers. In the latter case, a first mass analyzer can be use to separate the protein ions (precursor ion) from each other and determine the molecular weights of the various protein constituents in the sample. A second mass analyzer can be used to analyze each separated constituents, e.g., by fragmenting the precursor ions into product ions by using, e.g. an inert gas. Any desired combination of mass analyzers can be used, including, e.g., triple quadrupoles, tandem time-of-flights, ion traps, and/or combinations thereof.

Different kinds of detectors can be used to detect the protein ions. For example, destructive detectors can be utilized, such as ion electron multipliers or cryogenic detectors (e.g., U.S. Pat. No. 5,640,010). Additionally, non-destructive detectors can be used, such as ion traps which are used as ion current pick-up devices in quadrupole ion trap mass analyzers or FTMS.

For MALDI-TOF, a number of sample preparation methods can be utilized including, dried droplet (Karasand Hillenkamp, Anal. Chem., 60:2299-2301, 1988), vacuum-drying (Winberger et al., In Proceedings of the 41st ASMS Conference on Mass Spectrometry and Allied Topics, San Francisco, May 31-Jun. 4, 1993, pp. 775a-b), crush crystals (Xiang et al., Rapid Comm. Mass Spectrom., 8:199-204, 1994), slow crystal growing (Xiang et al., Org. Mass Spectrom, 28:1424-1429, 1993); active film (Mock et al., Rapid Comm. Mass Spectrom., 6:233-238, 1992; Bai et al., Anal. Chem., 66:3423-3430, 1994), pneumatic spray (Kochling et al., Proceedings of the 43rd ASMS Conference on Mass Spectrometry and Allied Topics; Atlanta, Ga., May 21-26, 1995, p 1225); electrospray (Hensel et al., Proceedings of the 43rd ASMS Conference on Mass Spectrometry and Allied Topics; Atlanta, Ga., May 21-26, 1995, p 947); fast solvent evaporation (Vorm et al., Anal. Chem., 66:3281-3287, 1994); sandwich (Li et al., J. Am. Chem. Soc., 11 8:11662-11663, 1996); and two-layer methods (Dal et al., Anal. Chem., 71:1087-1091, 1999). See also, e.g., Liang et al., Rapid Commun. Mass Spectrom., 10: 1219-1226, 1996; van Adrichem et al., Anal. Chem., 70:923-930, 1998.

For MALDI analysis, samples are prepared as solid-state co-crystals or thin films by mixing them with an energy absorbing compound or colloid (the matrix) in the liquid phase, and ultimately drying the solution to the solid state upon the surface of an inert probe. In some cases an energy absorbing molecule (EAM) is an integral component of the sample presenting surface. Regardless of EAM application strategy, the probe contents are allowed to dry to the solid state prior to introduction into the laser desorption/ionization time-of-flight mass spectrometer (LDIMS).

Ion detection in TOF mass spectrometry is typically achieved with the use of electro-emissive detectors such as electron multipliers (EMP) or microchannel plates (MCP). Both of these devices function by converting primary incident charged particles into a cascade of secondary, tertiary, quaternary, etc. electrons. The probability of secondary electrons being generated by the impact of a single incident charged particle can be taken to be the ion-to-electron conversion efficiency of this charged particle (or more simply, the conversion efficiency). The total electron yield for cascading events when compared to the total number of incident charged particles is typically described as the detector gain. Because generally the overall response time of MCPs is far superior to that of EMPs, MCPs are the preferred electro-emissive detector for enhancing mass/charge resolving power. However, EMPs function well for detecting ion populations of disbursed kinetic energies, where rapid response time and broad frequency bandwidth are not necessary.

In a preferred aspect, for the analysis of digested proteins, a liquid-chromatography tandem mass spectrometer (LC-TMS) is used. This system provides an additional stage of sample separation via use of a liquid chromatograph followed by tandem mass spectrometry.

In preferred aspects, a protein eluted from a column according to the system described in Example 1 is analyzed using both MS and MS-MS analysis. For example, a small portion of intact proteins eluting from RP2 may be diverted to online detection using LC-ESI MS. The proteins are aliquoted on a number of plates allowing digestion or not with trypsin, preparation for MALDI-MS as well as for ESI-MS, as well as preparation of the MALDI plates with different matrices. The methods thus allow, in addition to information on intact mass, to conduct an analysis by both peptide mass fingerprinting and MS-MS techniques.

The methods described herein of separating and fractionating proteins provide individual proteins or fractions containing small numbers of distinct proteins. These proteins can be identified by mass spectral determination of the molecular masses of the protein and peptides resulting from the fragmentation thereof. Making use of available information in protein sequence databases, a comparison can be made between proteolytic peptide mass patterns generated in silicon and experimentally observed peptide masses. A “hit-list” can be compiled, ranking candidate proteins in the database, based on (among other criteria) the number of matches between the theoretical and experimental proteolytic fragments. Several Web sites are accessible that provide software for protein identification on-line, based on peptide mapping and sequence database search strategies (e.g., http://www.expasy.ch). Methods of peptide mapping and sequencing using MS are described in WO 95/252819, U.S. Pat. No. 5,538,897, U.S. Pat. No. 5,869,240, U.S. Pat. No. 5,572,259, and U.S. Pat. No. 5,696,376. See, also, Yates, J. Mass Spec., 33:1 (1998).

Data collected from a mass spectrometer typically comprises the intensity and mass to charge ratio for each detected event. Spectral data can be recorded in any suitable form, including, e.g., in graphical, numerical, or electronic formats, either in digital or analog form. Spectra are preferably recorded in a storage medium, including, e.g., magnetic, such as floppy disk, tape, or hard disk; optical, such as CD-ROM or laser-disc; or, ROM-CHIPS.

The mass spectrum of a given sample typically provides information on protein intensity, mass to charge ratio, and molecular weight. In preferred embodiments of the invention, the molecular weights of proteins in the sample are used as a matching criterion to query a database. The molecular weights are calculated conventionally, e.g., by subtracting the mass of the ionizing proton for singly-charged protonated molecular ions, by multiplying the measured mass/charge ratio by the number of charges for multiply-charged ions and subtracting the number of ionizing protons.

Various databases are useful in accordance with the present invention. Useful databases include, databases containing genomic sequences, expressed gene sequences, and/or expressed protein sequences. Preferred databases contain nucleotide sequence-derived molecular masses of proteins present in a known organism, organ, tissue, or cell-type. There are a number of algorithms to identify open reading frames (ORF) and convert nucleotide sequences into protein sequence and molecular weight information. Several publicly accessible databases are available, including, the SwissPROT/TrEMBL database (http://www.expasy.ch).

Typically, a mass spectrometer is equipped with commercial software that identifies peaks above a certain threshold level, calculates mass, charge, and intensity of detected ions. Correlating molecular weight with a given output peak can be accomplished directly from the spectral data, i.e., where the charge on an ion is one and the molecular weight is therefore equal to the numerator value minus the mass of the ionizing proton. However, protein ions can be complexed with various counter-ions and adducts, such as N, C, and K′. In such a case, it would be expected that a given protein ion would exhibit multiple peaks, such as a triplet, representing different ionic states (or species) of the same protein. Thus, it may be necessary to analyze and process spectral data to determine families of peaks arising from the same protein. This analysis can be carried out conventionally, e.g., as described by Mann et al., anal. Chem., 61:1702-1708 (1989).

In matching a molecular mass calculated from a mass spectrometer to a molecular mass predicted from a database, such as a genomic or expressed gene database, post-translation processing may have to be considered. There are various processing events which modify protein structure, including, proteolytic processing, removal of N-terminal methionine, acetylation, methylation, glycosylation, phosphorylation, etc.

A database can be queried for a range of proteins matching the molecular mass of the unknown. The range window can be determined by the accuracy of the instrument, the method by which the sample was prepared, etc. Based on the number of hits (where a hit is match) in the spectrum, the unknown protein or peptide is identified or classified.

Methods of identifying one or more CPP by mass spectrometry are useful for diagnosis and prognosis of cardiovascular disorders. Preferably, such methods are used to detect one or more CPP present in human plasma. Exemplary techniques are described in U.S. patent application Ser. Nos. 02/0060290, 02/0137106, 02/0138208, 02/0142343, 02/0155509, disclosures of which are incorporated by reference in their entireties.

Diagnostic and Prognostic Uses

The nucleic acid molecules, proteins, protein homologues, and antibodies described herein can be used in one or more of the following methods: diagnostic assays, prognostic assays, monitoring clinical trials, and pharmacogenetics; and in drug screening and methods of treatment (e.g., therapeutic and prophylactic) as further described herein.

The invention provides diagnostic and prognostic assays for detecting CPP nucleic acids and proteins, as further described. Also provided are diagnostic and prognostic assays for detecting interactions between CPPs and CPP target molecules, particularly natural agonists and antagonists.

The present invention provides methods for identifying polypeptides that are differentially expressed between two or more samples. “Differential expression” refers to differences in the quantity or quality of a polypeptide between samples. Such differences could result at any stage of protein expression from transcription through post-translational modification. For example, using protein array methods, two samples are bound to affinity spots on different sets of adsorbents (e.g., chips) and recognition maps are compared to identify polypeptides that are differentially retained by the two sets of adsorbents. Differential retention includes quantitative retention as well as qualitative differences in the polypeptide. For example, differences in post-translational modification of a protein can result in differences in recognition maps detectable as differences in binding characteristics (e.g., glycosylated proteins bind differently to lectin adsorbents) or differences in mass (e.g., post-translational cleavage products). In certain embodiments, an adsorbent can have an array of affinity spots selected for a combination of markers diagnostic for a disease or syndrome.

Differences in polypeptide levels between samples (e.g., differentially expressed CPPs in plasma samples) can be identified by exposing the samples to a variety of conditions for analysis by desorption spectrometry (e.g., mass spectrometry). Unknown proteins can be identified by detecting physicochemical characteristics (e.g., molecular mass), and this information can be used to search databases for proteins having similar profiles.

Preferred methods of detecting a CPP utilize mass spectrometry techniques. Such methods provide information about the size and character of the particular CPP isoform that is present in a sample, e.g., a biological sample submitted for diagnosis or prognosis. Mass spectrometry techniques are detailed in the section titled “detection of CPPs by mass spectrometry”. Example 1 outlines a preferred detection scheme, wherein a biological sample is separated by chromatography before characterization by mass spectrometry. The invention provides a method of detecting a CPP in a biological sample comprising the steps of: fractionating a biological sample (e.g., plasma, serum, lymph, cerebrospinal fluid, cell lysate of a particular tissue) by at least one chromatographic step; subjecting a fraction to mass spectrometry; and comparing the characteristics of polypeptide species observed in mass spectrometry with known characteristics of CPP polypeptides (e.g., CPP 22, as disclosed in Table 1).

The isolated nucleic acid molecules of the invention can be used, for example, to detect CPP mRNA (e.g., in a biological sample) or a genetic alteration in a CPP-encoding gene, and to modulate a CPP activity, as described further below. The CPP can be used to treat disorders characterized by insufficient production of a CPP or by excessive production of a CPP target molecule. In addition, the CPPs can be used to screen for naturally occurring CPP target molecules, to screen for drugs or compounds which modulate CPP activity. Moreover, the anti-CPP antibodies of the invention can be used to detect and isolate CPPs, regulate the bioavailability of CPPs, and modulate CPP activity.

Accordingly one embodiment of the present invention involves a method of use (e.g., a diagnostic assay, prognostic assay, or a prophylactic/therapeutic method of treatment) wherein a molecule of the present invention (e.g., a CPP, CPP nucleic acid, or CPP modulator) is used, for example, to diagnose, prognose and/or treat a disorder in which any of the aforementioned CPP activities is indicated. In another embodiment the present invention involves a method of use (e.g., a diagnostic assay, prognostic assay, or a prophylactic/therapeutic method of treatment) wherein a molecule of the present invention is used, for example, for the diagnosis, prognosis, and/or treatment of subjects, preferably human subjects, in which any of the aforementioned activities is pathologically perturbed. In a preferred embodiment, the methods of use involve administering to a subject preferably a human subject, a molecule of the present invention for the diagnosis, prognosis, and/or therapeutic treatment. In another embodiment, the methods of use involve administering to a human subject a molecule of the present invention.

For example, the invention encompasses a method of determining whether a CPP is expressed within a biological sample comprising: a) contacting said biological sample with: i) a polynucleotide that hybridizes under stringent conditions to a CPP nucleic acid; or ii) a detectable polypeptide (e.g. antibody) that selectively binds to a CPP; and b) detecting the presence or absence of hybridization between said polynucleotide and an RNA species within said sample, or the presence or absence of binding of said detectable polypeptide to a polypeptide within said sample. Detection of said hybridization or of said binding indicates that said CPP is expressed within said sample. Preferably, the polynucleotide is a primer, and said hybridization is detected by detecting the presence of an amplification product comprising said primer sequence, or the detectable polypeptide is an antibody.

In certain embodiments, detection involves the use of a probe/primer in a polymerase chain reaction (PCR) (see, e.g., U.S. Pat. Nos. 4,683,195 and 4,683,202, the disclosures of which are incorporated herein by reference in their entireties), such as anchor PCR or RACE PCR, or, alternatively, in a ligation chain reaction (LCR) (see, e.g., Landegren et al. (1988) Science 241:1077-1080; and Nakazawa et al. (1994) PNAS 91:360-364, the disclosures of which are incorporated herein by reference in their entireties), the latter of which can be particularly useful for detecting point mutations in the CPP-encoding-gene (see Abravaya et al. (1995) Nucleic Acids Res. 23:675-682, the disclosure of which is incorporated herein by reference in its entirety).

Also envisioned is a method of determining whether a mammal, preferably human, has an elevated or reduced level of expression of a CPP, comprising: a) providing a biological sample from said mammal; and b) comparing the amount of a CPP or of a CPP RNA species encoding a CPP within said biological sample with a level detected in or expected from a control sample. An increased amount of said CPP or said CPP RNA species within said biological sample compared to said level detected in or expected from said control sample indicates that said mammal has an elevated level of CPP expression, and a decreased amount of said CPP or said CPP RNA species within said biological sample compared to said level detected in or expected from said control sample indicates that said mammal has a reduced level of expression of a CPP.

The present invention also pertains to the field of predictive medicine in which diagnostic assays, prognostic assays, and monitoring clinical trials are used for prognostic purposes to thereby treat an individual prophylactically. Accordingly, one aspect of the present invention relates to diagnostic assays for determining CPP and/or nucleic acid expression as well as CPP activity, in the context of a biological sample (e.g., blood, plasma, cells, tissue) to thereby determine whether an individual is afflicted with a disease or disorder, or is at risk of developing a disorder, associated with aberrant CPP expression or activity. The invention also provides for prognostic (or predictive) assays for determining whether an individual is at risk of developing a disorder associated with a CPP, nucleic acid expression or activity. For example, mutations in a CPP-encoding gene can be assayed in a biological sample. Such assays can be used for prognostic or predictive purpose to thereby prophylactically treat an individual prior to the onset of a disorder characterized by or associated with CPP expression or activity.

The term “biological sample” is intended to include tissues, cells and biological fluids isolated from an individual, as well as tissues, cells and fluids present within an individual. That is, the detection methods of the invention can be used to detect a CPP mRNA, protein, or genomic DNA in a biological sample in vitro as well as in vivo. Preferred biological samples are biological fluids such as lymph, cerebrospinal fluid, blood, and especially blood plasma. For example, in vitro techniques for detection of a CPP mRNA include Northern hybridizations and in situ hybridizations. In vitro techniques for detection of a CPP include mass spectrometry, Enzyme Linked Immuno Sorbent Assays (ELISAs), Western blots, immunoprecipitations and immunofluorescence. In vitro techniques for detection of a CPP-encoding genomic DNA include Southern hybridizations. Furthermore, in vivo techniques for detection of a CPP include introducing into an individual a labeled anti-CPP antibody.

In preferred embodiments, the subject methods can be characterized by generally comprising detecting, in a tissue sample of the individual (e.g. a human patient), the presence or absence of a genetic lesion characterized by at least one of (i) a mutation of a gene encoding one of the subject CPP or (ii) the mis-expression of a CPP-encoding gene. To illustrate, such genetic lesions can be detected by ascertaining the existence of at least one of (i) a deletion of one or more nucleotides from the CPP-encoding gene, (ii) an addition of one or more nucleotides to the gene, (iii) a substitution of one or more nucleotides of the gene, (iv) a gross chromosomal rearrangement or amplification of the gene, (v) a gross alteration in the level of a messenger RNA transcript of the gene, (vi) aberrant modification of the gene, such as of the methylation pattern of the genomic DNA, (vii) the presence of a non-wild type splicing pattern of a messenger RNA transcript of the gene, and (viii) reduced level of expression, indicating lesion in regulatory element or reduced stability of a CPP-encoding transcript.

In yet another exemplary embodiment, aberrant methylation patterns of a CPP nucleic acid can be detected by digesting genomic DNA from a patient sample with one or more restriction endonucleases that are sensitive to methylation and for which recognition sites exist in the CPP-encoding gene (including in the flanking and intronic sequences). See, for example, Buiting et al. (1994) Human Mol Genet 3:893-895. Digested DNA is separated by gel electrophoresis, and hybridized with probes derived from, for example, genomic or cDNA sequences. The methylation status of the CPP-encoding gene can be determined by comparison of the restriction pattern generated from the sample DNA with that for a standard of known methylation.

In yet another embodiment, a diagnostic assay is provided which detects the ability of a CPP to bind to a cell surface or extracellular protein. For instance, it will be desirable to detect CPP mutants which, while expressed at appreciable levels in the cell, are defective at binding a CPP target protein (having either diminished or enhanced binding affinity for the target). Such mutants may arise, for example, from mutations, e.g., point mutants, which may be impractical to detect by the diagnostic DNA sequencing techniques or by the immunoassays described above. The present invention accordingly further contemplates diagnostic screening assays which generally comprise cloning one or more CPP-encoding gene from the sample tissue, and expressing the cloned genes under conditions which permit detection of an interaction between that recombinant gene product and a target protein. As will be apparent from the description of the various drug screening assays set forth herein, a wide variety of techniques can be used to determine the ability of a CPP to bind to other components. These techniques can be used to detect mutations in a CPP-encoding gene which give rise to mutant proteins with a higher or lower binding affinity for a CPP target protein relative to the wild-type CPP. Conversely, by switching which of the CPP target protein and CPP is the “bait” and which is derived from the patient sample, the subject assay can also be used to detect CPP target protein mutants which have a higher or lower binding affinity for a CPP relative to a wild type form of that CPP target protein.

In an exemplary embodiment, a target protein can be provided as an immobilized protein (a “target”), such as by use of GST fusion proteins and glutathione treated microtiter plates as described herein.

In another embodiment, the methods further involve obtaining a control biological sample from a control subject, contacting the control sample with a compound or agent capable of detecting a CPP, mRNA, or genomic DNA, such that the level of a CPP, mRNA or genomic DNA is measured in the biological sample, and comparing the level of the CPP, mRNA or genomic DNA in the control sample to the level in the test sample. The invention also encompasses kits for detecting the presence of a CPP, mRNA or genomic DNA in a biological sample. For example, the kit can comprise: a labeled compound or agent capable of detecting a CPP, mRNA or genomic DNA in a biological sample; means for determining the amount of a CPP in the sample; and means for comparing the amount of CPP in the sample with a standard. The compound or agent can be packaged in a suitable container. The kit can further comprise instructions for using the kit to detect CPP or nucleic acid.

Drug Screening Assays

The invention provides a method (also referred to herein as a “screening assay”) for identifying candidate modulators (e.g., small molecules, peptides, antibodies, peptidomimetics or other drugs) which bind to CPPs, have a modulatory effect on, for example, CPP expression or preferably CPP biological activity, or have a modulatory effect on, for example, the activity of a CPP target molecule. In some embodiments small molecules can be generated using combinatorial chemistry or can be obtained from a natural products library. Assays may be cell based or non-cell based assays. Drug screening assays may be binding assays or more preferentially functional assays, as further described.

When the invention is used for drug development, e.g., to determine the ability of a CPP or candidate modulator to induce an anti-cardiovascular disorder response, the body fluid analyzed for the level of at least one CPP is preferably from a non-human mammal. The non-human mammal is preferably one in which the induction of an anti-cardiovascular disorder response by endogenous and/or exogenous agents is predictive of the induction of such a response in a human. Rodents (mice, rats, etc.) and primates are particularly suitable for use in this aspect of the invention.

Agents that are found, using screening assays as further described herein, to modulate CPP activity by at least 5%, more preferably by at least 10%, still more preferably by at least 30%, still more preferably by at least 50%, still more preferably by at least 70%, even more preferably by at least 90%, may be selected for further testing as a prophylactic and/or therapeutic anti-cardiovascular disease agent.

In another aspect, agents that are found, using screening assays as further described herein, to modulate CPP expression by at least 5%, more preferably by at least 10%, still more preferably by at least 30%, still more preferably by at least 50%, still more preferably by at least 70%, even more preferably by at least 90%, may be selected for further testing as a prophylactic and/or therapeutic anti-cardiovascular disease agent.

Agents that are found to modulate CPP activity may be used, for example, to modulate treatment regimens for cardiovascular disorders or to reduce the symptoms of a cardiovascular disorder alone or in combination with other appropriate agents or treatments.

Protein array methods are useful for screening and drug discovery. For example, one member of a receptor/ligand pair is docked to an adsorbent, and its ability to bind the binding partner is determined in the presence of the test substance. Because of the rapidity with which adsorption can be tested, combinatorial libraries of test substances can be easily screened for their ability to modulate the interaction. In preferred screening methods, CPPs are docked to the adsorbent. Binding partners are preferably labeled, thus enabling detection of the interaction.

Alternatively, in certain embodiments, a test substance is docked to the adsorbent. The polypeptides of the invention are exposed to the test substance and screened for binding.

In other embodiments, an assay is a cell-based assay in which a cell which expresses a CPP or biologically active portion thereof is contacted with a test compound and the ability of the test compound to modulate CPP activity determined. Determining the ability of the test compound to modulate CPP activity can be accomplished by monitoring the bioactivity of the CPP or biologically active portion thereof. The cell, for example, can be of mammalian origin, insect origin, bacterial origin or a yeast cell.

In one embodiment, the invention provides assays for screening candidate or test compounds which are target molecules of a CPP or biologically active portion thereof. In another embodiment, the invention provides assays for screening candidate or test compounds which bind to or modulate the activity of a CPP or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is used with peptide libraries, while the other four approaches are applicable to peptide, non-peptide oligomer or small molecule libraries of compounds (Lam, K. S. (1997) Anticancer Drug Des. 12:145, the disclosure of which is incorporated herein by reference in its entirety).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059 and 2061; and in Gallop et al. (1994) J. Med. Chem. 37:1233, the disclosures of which are incorporated herein by reference in their entireties.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devin (1990) Science 249:404-406); (Cwirla et al. (1990) Proc. Natl. Acad. Sci. 87:6378-6382); (Felici (1991) J. Mol. Biol. 222:301-310); (Ladner supra).

Determining the ability of the test compound to modulate CPP activity can also be accomplished, for example, by coupling the CPP or biologically active portion thereof with a label group such that binding of the CPP or biologically active portion thereof to its cognate target molecule can be determined by detecting the labeled CPP or biologically active portion thereof in a complex. For example, the extent of complex formation may be measured by immunoprecipitating the complex or by performing gel electrophoresis.

It is also within the scope of this invention to determine the ability of a compound to interact with its cognate target molecule without the labeling of any of the interactants. For example, a microphysiometer can be used to detect the interaction of a compound with its cognate target molecule without the labeling of either the compound or the target molecule. McConnell, H. M. et al. (1992) Science 257:1906-1912, the disclosure of which is incorporated by reference in its entirety. A microphysiometer such as a cytosensor is an analytical instrument that measures the rate at which a cell acidifies its environment using a Light-Addressable Potentiometric Sensor (LAPS). Changes in this acidification rate can be used as an indicator of the interaction between compound and receptor.

In a preferred embodiment, the assay comprises: contacting a cell which expresses a CPP or biologically active portion thereof with a target molecule to form an assay mixture, contacting the assay mixture with a test compound, and determining the ability of the test compound to modulate the activity of the CPP or biologically active portion thereof. Determining the ability of the test compound to modulate the activity of the CPP or biologically active portion thereof comprises: determining the ability of the test compound to modulate a biological activity of the CPP expressing cell (e.g., interaction with a CPP target molecule, as discussed above).

In another preferred embodiment, the assay comprises contacting a cell which is responsive to a CPP or biologically active portion thereof with a CPP or biologically active portion thereof, to form an assay mixture, contacting the assay mixture with a test compound, and determining the ability of the test compound to modulate the activity of the CPP or biologically active portion thereof. Determining the ability of the test compound to modulate the activity of the CPP or biologically active portion thereof comprises determining the ability of the test compound to modulate a biological activity of the CPP-responsive cell.

In another embodiment, an assay is a cell-based assay comprising contacting a cell expressing a CPP target molecule (i.e. a molecule with which CPPs interact) with a test compound and determining the ability of the test compound to modulate the activity of the CPP target molecule. Determining the ability of the test compound to modulate the activity of a CPP target molecule can be accomplished, for example, by assessing the activity of a target molecule, or by assessing the ability of the CPP to bind to or interact with the CPP target molecule.

Determining the ability of the CPP to bind to or interact with a CPP target molecule, for example, can be accomplished by one of the methods described above for directly or indirectly determining binding. In a preferred embodiment, the assay includes contacting the CPP or biologically active portion thereof with a known compound which binds said CPP (e.g., a CPP antibody or target molecule) to form an assay mixture, contacting the CPP with a test compound before or after said known compound, and determining the ability of the test compound to interact with the CPP. Determining the ability of the test compound to interact with a CPP comprises determining the ability of the test compound to preferentially bind to the CPP or biologically active portion thereof as compared to the known compound. Determining the ability of the CPP to bind to a CPP target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705, the disclosures of which are incorporated herein by reference in their entireties. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, the assay is a cell-free assay in which a CPP or biologically active portion thereof is contacted with a test compound and the ability of the test compound to modulate the activity of the CPP or biologically active portion thereof is determined. In a preferred embodiment, determining the ability of the CPP to modulate or interact with a CPP target molecule can be accomplished by determining the activity of the target molecule. For example, the activity of the target molecule can be determined by contacting the target molecule with the CPP or a fragment thereof and measuring induction of a cellular second messenger of the target (e.g., cAMP, STAT3, Akt, intracellular Ca2+, diacylglycerol, IP3, etc.), detecting catalytic/enzymatic activity of the target for an appropriate substrate, detecting the induction of a reporter gene (comprising a target-responsive regulatory element operatively linked to a nucleic acid encoding a detectable marker, e.g., luciferase), or detecting a target-regulated cellular response, for example, signal transduction or protein:protein interactions.

The cell-free assays of the present invention are amenable to use of both soluble and/or membrane-bound forms of isolated proteins (e.g. CPPs or biologically active portions thereof or molecules to which CPPs targets bind). In the case of cell-free assays in which a membrane-bound form an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton™ X-100, Triton™ X-114, Thesit™, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either a CPP or its target molecule to facilitate separation of complexed from uncomplexed forms of one or both of the proteins, as well as to accommodate automation of the assay. Binding of a test compound to a CPP, or interaction of a CPP with a target molecule in the presence and absence of a candidate compound, can be accomplished in any vessel suitable for containing the reactants and by any immobilization protocol described herein. Alternatively, the complexes can be dissociated from the matrix, and the level of CPP binding or activity determined using standard techniques.

Other techniques for immobilizing proteins on matrices can also be used in the screening assays of the invention. For example, either a CPP or a CPP target molecule can be immobilized utilizing conjugation of biotin and streptavidin. Biotinylated CPP or target molecules can be prepared from biotin-NHS(N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with CPP or target molecules but which do not interfere with binding of the CPP to its target molecule can be derivatized to the wells of the plate, and unbound target or CPP trapped in the wells by antibody conjugation. Methods for detecting such complexes, in addition to those described above for the GST-immobilized complexes, include immunodetection of complexes using antibodies reactive with the CPP or target molecule, as well as enzyme-linked assays which rely on detecting an enzymatic activity associated with the CPP or target molecule.

In another embodiment, modulators of CPP expression are identified in a method wherein a cell is contacted with a candidate compound and the expression of CPP mRNA or protein in the cell is determined. The level of expression of CPP mRNA or protein in the presence of the candidate compound is compared to the level of expression of CPP mRNA or protein in the absence of the candidate compound. The candidate compound can then be identified as a modulator of CPP expression based on this comparison. For example, when expression of CPP mRNA or protein is greater (statistically significantly greater) in the presence of the candidate compound than in its absence, the candidate compound is identified as a stimulator of CPP mRNA or protein expression. Alternatively, when expression of CPP mRNA or protein is less (statistically significantly less) in the presence of the candidate compound than in its absence, the candidate compound is identified as an inhibitor of CPP mRNA or protein expression. The level of CPP mRNA or protein expression in the cells can be determined by methods described herein for detecting CPP mRNA or protein.

In yet another aspect of the invention, the CPP can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300, the disclosures of which are incorporated herein by reference in their entireties), to identify other proteins, which bind to or interact with CPPs (“CPP-binding proteins” or “CPP-bp”) and are involved in CPP activity. Such CPP-binding proteins are also likely to be involved in the propagation of signals by the CPP or CPP targets as, for example, downstream elements of a CPP-mediated signaling pathway.

The two-hybrid system is based on the modular nature of most transcription factors, which consist of separable DNA-binding and activation domains. Briefly, the assay utilizes two different DNA constructs. In one construct, the gene that codes for a CPP or a fragment thereof is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a CPP-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the CPP.

This invention further pertains to novel agents identified by the above-described screening assays and to processes for producing such agents by use of these assays. Accordingly, in one embodiment, the present invention includes a compound or agent obtainable by a method comprising the steps of any one of the aforementioned screening assays (e.g., cell-based assays or cell-free assays).

Accordingly, it is within the scope of this invention to further use an agent identified as described herein in an appropriate animal model. For example, an agent identified as described herein (e.g., a CPP modulating agent, or a CPP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention also pertains to uses of novel agents identified by the above-described screening assays for diagnoses, prognoses, prevention, and treatments as described herein. Accordingly, it is within the scope of the present invention to use such agents in the design, formulation, synthesis, manufacture, and/or production of a drug or pharmaceutical composition for use in diagnosis, prognosis, or treatment, as described herein. For example, in one embodiment, the present invention includes a method of synthesizing or producing a drug or pharmaceutical composition by reference to the structure and/or properties of a compound obtainable by one of the above-described screening assays. For example, a drug or pharmaceutical composition can be synthesized based on the structure and/or properties of a compound obtained by a method in which a cell which expresses a CPP target molecule is contacted with a test compound and the ability of the test compound to bind to, or modulate the activity of, the CPP target molecule is determined. In another exemplary embodiment, the present invention includes a method of synthesizing or producing a drug or pharmaceutical composition based on the structure and/or properties of a compound obtainable by a method in which a CPP or biologically active portion thereof is contacted with a test compound and the ability of the test compound to bind to, or modulate the activity of, the CPP or biologically active portion thereof is determined

Animal Based Drug Screening

It is also advantageous to carry out drug screening assays in vivo. In vivo screening assays are carried out in nonhuman animals to discover effective CPP modulators that may play a role in cardiovascular disease. Animal-based model systems of cardiovascular disease include, but are not limited to, non-recombinant animals and transgenic animals.

Non-recombinant animal models for cardiovascular disease may include, for example, genetic models. Such genetic cardiovascular disease models include apoB or apoR deficient pigs (Rapacz, et al., 1986, Science 234:1573-1577) and Watanabe heritable hyperlipidemic (WHHL) rabbits (Kita et al., 1987, Proc. Natl. Acad. Sci U.S.A. 84: 5928-5931). Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty, for example.

As indicated in the art (Ferns, G. A. A. et al. (1991) Science, 253:1129-1132) the rat carotid artery injury model of restenosis can be a useful indication of potential therapeutic action. An example of this method is described in U.S. Pat. No. 6,500,859, the disclosure of which is incorporated herein by reference. Briefly, the protocol approved by the National Institute on Aging Animal Care and use Committee used 6 month Wistar rats from the GRC colony anesthetized with 20 mg/kg body weight pentobarbital, 2 mg/kg body weight ketamine, and 4 mg/kg body weight xylazine intraperitoneally. The left external carotid artery was cannulated with 2-French Fogarty embolectomy catheter, inflated with saline and passed three times up and down the common carotid artery to produce a distending, deendothelializing injury. The animals were treated with an appropriate dosage of the test substance or with vehicle alone (e.g., based on body weight per day in an appropriate solution such as 1:2:2:165 DMSO:Cremophor EL:Dehydrated ethanol:phosphate buffered saline) by intraperitoneal injection beginning 2 hours after injury. Test substance or vehicle alone was administered once daily, as an intraperitoneal injection, for the next 4 days. After 11 days the animals (8 treated and 10 vehicle-treated) were anesthetized as above and the carotid artery was isolated and fixed in 10% buffered formalin and embedded in paraffin. Cross sections of the carotids were mounted on microscope slides and stained with hematoxylin and eosin stain. The image of the carotid artery was projected onto a digitizing board and the cross sectional areas of the intima and the media were measured. Reduction of the neointimal area (thickening) indicates that the test substance is an effective antirestenosis agent.

Interfering with the recirculation of bile acids from the lumen of the intestinal tract is found to reduce the levels of serum cholesterol in a causal relationship. Epidemiological data has accumulated which indicates such reduction leads to an improvement in the disease state of atherosclerosis (Stedronsky, Biochimica et Biophysica Acta, 1210, 255-287 (1994)). Inhibition of cholesteryl ester transfer protein (CETP) has been shown to effectively modify plasma HDL/LDL ratios, and is expected to check the progress and/or formation of certain cardiovascular diseases. Inhibition of CETP should lead to elevation of plasma HDL cholesterol and lowering of plasma LDL cholesterol, thereby providing a therapeutically beneficial plasma lipid profile (McCarthy, Medicinal Res. Revs., 13, 139-59 (1993)). An in vivo assay for compounds that inhibit rat ileal uptake of ¹⁴C-Taurocholate into bile (CETP inhibition) is disclosed in U.S. Pat. No. 6,489,366 and Une, et al. Biochimica et Biophysica Acta, 833, 196-202 (1985), disclosures of which are incorporated herein by reference.

Briefly, male Wistar rats (200-300 g) are anesthetized with inactin (100 mg/kg). Bile ducts are cannulated with a 10 inch length of PE10 tubing. The small intestine is to be exposed and laid out on a gauze pad. A canulae (⅛″ luer lock, tapered female adapter) is inserted at 12 cm from the junction of the small intestine and the cecum. A slit is cut at 4 cm from this same junction (utilizing a 8 cm length of ileum). Twenty milliliters of warm Dulbecco's phosphate buffered saline, pH 6.5 (PBS) is to be used to flush out the intestine segment. The distal opening is cannulated with a 20 cm length of silicone tubing (0.02″ LD.times.0.037″ O.D.). The proximal cannulae is hooked up to a peristaltic pump and the intestine is washed for 20 min with warm PBS at 0.25 ml/min. Temperature of the gut segment is to be monitored continuously. At the start of the experiment, 2.0 ml of control sample (¹⁴C-taurocholate at 0.05 mCi/mL with 5 mM non-radiolabeled taurocholate) is loaded into the gut segment with a 3 ml syringe and bile sample collection is begun. Control sample is infused at a rate of 0.25 ml/min for 21 min. Bile samples fractions are to be collected every 3 minute for the first 27 minutes of the procedure. After the 21 min of sample infusion, the ileal loop is to be washed out with 20 ml of warm PBS (using a 30 ml syringe), and then the loop is to be washed out for 21 min with warm PBS at 0.25 ml/min. A second perfusion is initiated as described above but this with test compound being administered as well (21 min administration followed by 21 min of wash out) and bile sampled every 3 min for the first 27 min. If necessary, a third perfusion is performed as above that typically contains the control sample.

In addition, measurement of hepatic cholesterol concentration is a useful assay for determining the effectiveness of a test substance against cardiovascular disorders. In this assay, liver tissue is weighed and homogenized in chloroform:methanol (2:1). After homogenization and centrifugation the supernatant is separated and dried under nitrogen. The residue is to be dissolved in isopropanol and the cholesterol content measured enzymatically, using a combination of cholesterol oxidase and peroxidase, as described by Allain, C. A. et al., Clin. Chem., 20, 470 (1974) (herein incorporated by reference).

Similarly, serum cholesterol may be determined as follows. Total serum cholesterol is measured enzymatically using a commercial kit from Wako Fine Chemicals (Richmond, Va.); Cholesterol C11, Catalog No. 276-64909. HDL cholesterol may be assayed using this same kit after precipitation of VLDL and LDL with Sigma Chemical Co. HDL Cholesterol reagent, Catalog No. 352-3 (dextran sulfate method). Total serum triglycerides (blanked) (TGI) is also assayed enzymatically with Sigma Chemical Co. GPO-Trinder, Catalog No. 337-B. VLDL and LDL (VLDL+LDL) cholesterol concentrations are calculated as the difference between total and HDL cholesterol. A reduction in VLDL+LDL cholesterol in the test substance-treated sample relative to control is indicative of an effective anticardiovascular disorder agent.

A dog model for evaluating lipid lowering drugs may also be utilized, for example, as described in U.S. Pat. No. 6,489,366.

Briefly, male beagle dogs, obtained from a vendor such as Marshall farms and weighing 6-12 kg are fed once a day for two hours and given water ad libitum. Dogs may be randomly assigned to a dosing groups consisting of 6 to 12 dogs each, such as: vehicle, i.g.; 1 mg/kg, i.g.; 2 mg/kg, i.g.; 4 mg/kg, i.g.; 2 mg/kg, p.o. (powder in capsule). Intra-gastric dosing of a therapeutic material dissolved in aqueous solution (for example, 0.2% Tween 80 solution [polyoxyethylene mono-oleate, Sigma Chemical Co., St. Louis, Mo.]) may be done using a gavage tube. Prior to initiating dosing, blood samples may be drawn from the cephalic vein in the morning before feeding in order to evaluate serum cholesterol (total and HDL) and triglycerides. For several consecutive days animals are dosed in the morning, prior to feeding. Animals are to be allowed 2 hours to eat before any remaining food is removed. Feces are to be collected over a 2 day period at the end of the study and may be analyzed for bile acid or lipid content. Blood samples are also to be taken, at the end of the treatment period, for comparison with pre-study serum lipid levels. Statistical significance will be determined using the standard student's T-test with p<0.05.

Serum lipid measurement is measured similarly. Blood is collected from the cephalic vein of fasted dogs in serum separator tubes (Vacutainer SST, Becton Dickinson and Co., Franklin Lakes, N.J.). The blood is centrifuged at 2000 rpm for 20 minutes and the serum decanted. Total cholesterol may be measured in a 96 well format using a Wako enzymatic diagnostic kit (Cholesterol CII) (Wako Chemicals, Richmond, Va.), utilizing the cholesterol oxidase reaction to produce hydrogen peroxide which is measured calorimetrically. A standard curve from 0.5 to 10 ug cholesterol is to be prepared in the first 2 columns of the plate. The serum samples (20-40 ul, depending on the expected lipid concentration) or known serum control samples are added to separate wells in duplicate. Water is added to bring the volume to 100 ul in each well. A 100 ul aliquot of color reagent is added to each well and the plates will be read at 500 nm after a 15 minute incubation at 37 degrees centigrade.

HDL cholesterol may be assayed using Sigma kit No. 352-3 (Sigma Chemical Co., St. Louis, Mo.) which utilizes dextran sulfate and Mg ions to selectively precipitate LDL and VLDL. A volume of 150 ul of each serum sample is to be added to individual microfuge tubes, followed by 15 ul of HDL cholesterol reagent (Sigma 352-3). Samples are to be mixed and centrifuged at 5000 rpm for 5 minutes. A 50 ul aliquot of the supernatant is to be then mixed with 200 ul of saline and assayed using the same procedure as for total cholesterol measurement

Triglycerides are measured using Sigma kit No. 337 in a 96 well plate format. This procedure will measure glycerol, following its release by reaction of triglycerides with lipoprotein lipase. Standard solutions of glycerol (Sigma 339-11) ranging from 1 to 24 ug are to be used to generate the standard curve. Serum samples (20-40 ul, depending on the expected lipid concentration) are added to wells in duplicate. Water is added to bring the volume to 100 ul in each well and 100 ul of color reagent is also added to each well. After mixing and a 15 minute incubation, the plates will be read at 540 nm and the triglyceride values calculated from the standard curve. A replicate plate is also to be run using a blank enzyme reagent to correct for any endogenous glycerol in the serum samples.

Test compounds may be evaluated for their effect on serum glucose and serum insulin in db/db mice (C578BL/KsJ-db/db Jcl) as described in U.S. Pat. No. 6,462,046, disclosure of which is incorporated herein. The compounds are dissolved in a vehicle (e.g., consisting of 2% Tween80 in distilled water) and administered orally. Dosage is determined by body weight. All aspects of the work including experimentation and disposal of the animals is performed in general accordance with the International Guiding Principles for Biomedical Research Involving Animals (CIOMS Publication No. ISBN 92 90360194, 1985). Glucose-HA Assay kits (Wako, Japan) are used for determination of serum glucose and ELISA Mouse Insulin Assay kits (SPI bio, France) are utilized for determination of insulin. An appropriate positive control is troglitazone (Helios Pharmaceutical, Louisville, Ky.).

The animals are divided into twenty groups of four animals each. The animals weigh 52+/−5 gms at age 8-10 weeks. During the experiment the animals are provided free access to laboratory chow (Fwusow Industry Co., Taiwan) and water. Prior to any treatment a blood sample (pretreatment blood) is taken from each animal. Four groups of animals, the vehicle groups, receive only doses of the vehicle. Each of the vehicle groups receive 100, 30, 10 or 1 ml/kg body weight of the vehicle orally. A triglitazone solution (10 ml/kg body weight in tween 80/water) is administered orally to the four positive control groups in doses of 100, 30, 10 and 1 ml/kg body weight respectively. The test compound is similarly administered orally as a solution to four groups of animals with each group receiving a different dose of the compound. The vehicle, positive control and test compound solutions are administered to the groups immediately, 24 hours and 48 hours after drawing the pretreatment blood. Blood is withdrawn (post treatment blood) 1.5 hours after administration of the last dose. The serum glucose are determined enzymatically (Mutaratose-GOD) and the insulin levels by ELISA (mouse insulin assay kit). The mean SEM of each group is calculated and the percent inhibition of serum glucose and insulin obtained by comparison between pretreatment blood and post treatment blood. The percentage of reduction of the serum glucose and insulin levels in the post treatment blood relative to the pretreatment blood is determined and the Unpaired students t test applied for the comparison between the control and test solution groups and the vehicle group. A significant difference is considered at P<0.05. Troglitazone, as an effective anti-cardiovascular disorder agent, results in a reduced glucose level at 10 mg/kg body weight (25+/−2%).

U.S. Pat. No. 6,121,319, disclosure of which is incorporated herein, describes an assay for the progression of atherosclerosis in hypercholesterolemic rabbits. The rabbits are sacrificed and aortas obtained. The aortas are stained with sudan-4 and the extent of staining analyzed. The percent aortic surface area covered by lesions in test substance treated and untreated lipid-fed rabbits is graphed. The aortas of the rabbits treated with an effective anti-atherosclerotic agent have less staining, indicating decreased atherosclerosis. In addition, sections of the aortas are immunostained for VCAM-1 expression or macrophage accumulation using antibodies for VCAM-1 or Ram-11 antigen. Reduced VCAM-1 expression and macrophage accumulation compared to control treated samples are indicative of an effective agent.

Reduction in LDL cholesterol may also be determined in a primate model. For example, Cynomolgus monkeys are made hypercholesterolemic prior to test compound dosing by feeding a high fat cholesterol diet. The monkeys are then dosed orally with the test compound or control vehicle for two weeks. A reduction in the percentage serum LDL cholesterol in the monkeys over this time period is indicative of an effective anti-atherosclerotic agent.

Pharmaceutical Compositions

When polypeptides of the present invention are expressed in soluble form, for example as a secreted product of transformed yeast or mammalian cells, they can be purified according to standard procedures of the art, including steps of ammonium sulfate precipitation, ion exchange chromatography, gel filtration, electrophoresis, affinity chromatography, according to, e.g., “Enzyme Purification and Related Techniques,” Methods in Enzymology, 22:233-577 (1977), and Scopes, R., Protein Purification: Principles and Practice (Springer-Verlag, New York, 1982) provide guidance in such purifications. Likewise, when polypeptides of the invention are expressed in insoluble form, for example as aggregates or inclusion bodies, they can be purified by appropriate techniques, including separating the inclusion bodies from disrupted host cells by centrifugation, solubilizing the inclusion bodies with chaotropic and reducing agents, diluting the solubilized mixture, and lowering the concentration of chaotropic agent and reducing agent so that the polypeptide takes on a biologically active conformation. The latter procedures are disclosed in the following references, which are incorporated by reference: Winkler et al, Biochemistry, 25: 4041-4045 (1986); Winkler et al, Biotechnology, 3: 992-998 (1985); Koths et al, U.S. Pat. No. 4,569,790; and European patent applications 86306917.5 and 86306353.3.

Compounds capable of detecting or modulating a CPP or a CPP biological activity, including small molecules, peptides, CPP nucleic acid molecules, and anti-CPP antibodies of the invention, can be incorporated into pharmaceutical compositions suitable for administration. Such compositions typically comprise a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Where the active compound is a protein, e.g., an anti-CPP antibody, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and other required ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. For administration by inhalation, the compounds are delivered in the form of an aerosol spray from pressured container or dispenser which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Most preferably, active compound is delivered to a subject by intravenous injection.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811, the disclosure of which is incorporated herein by reference in its entirety.

In a further embodiment, the active compound may be coated on a microchip drug delivery device. Such devices are useful for controlled delivery of proteinaceous compositions into the bloodstream, cerebrospinal fluid, lymph, or tissue of an individual without subjecting such compositions to digestion or subjecting the individual to injection. Methods of using microchip drug delivery devices are described in U.S. Pat. Nos. 6,123,861, 5,797,898 and U.S. patent application 20020119176A1, disclosures of which are hereby incorporated in their entireties.

It is especially advantageous to formulate oral or preferably parenteral compositions in dosage unit form for ease of administration and uniformity of dosage. Dosage unit form as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating used to more accurately determine useful doses in humans. Levels in plasma may be measured, for example, by high performance liquid chromatography.

The pharmaceutical compositions can be included in a container, pack, or dispenser together with instructions for administration.

Cardiovascular Disorder Therapy

The CPPs, CPP modulators, and anti-CPP antibodies of the invention can be used in the treatment or prevention of CPP-related disorders. Unlike manufactured small molecule therapeutics that may cause an immune reaction or prove to be toxic to the host, the peptides of the invention are normally secreted and are thought not to be toxic to a subject to which they are administered. Thus, in one aspect the invention relates to pharmaceutical compositions containing a CPP as an active ingredient, preferably containing a pharmaceutically acceptable carrier or diluent. Another aspect relates to pharmaceutical compositions containing an antibody, antibody fragment, or peptide modulator of CPP, preferably containing a pharmaceutically acceptable carrier or diluent. The carrier or diluent is preferably adapted for oral, intravenous, intramuscular or subcutaneous administration. CPPs can also be prepared as solutions for inhalation. Pharmaceutical compositions may comprise or consist essentially of any of the CPPs, CPP modulators, anti-CPP antibodies, or anti-CPP antibody fragments described herein. Optionally, a CPP can be administered as a propeptide or prepropeptide.

A number of agents are useful for the treatment and prevention of cardiovascular disorders. Such agents may be used advantageously in combination with a CPP-related composition.

For example, cell cycle inhibitors and proto-oncogenes (Simari and Nabel, Semin. Intervent. Cardiol. 1:77-83 (1996)); NO (nitric oxide) donor drugs; pro-apoptotic agents such as bcl-x (Pollman et al., Nature Med. 2:222-227 (1998)); herpes virus thymidine kinase (tk) gene and systemic ganciclovir (Ohno et al., Science 265:781-784 (1994); Guzman et al., Proc. Natl. Acad. Sci. USA 91:10732-10736 (1994); Chang et al., Mol. Med. 1:172-181 (1995); and Simari et al., Circulation 92:1-501 (1995)) have been exploited to treat atherosclerosis, restenosis and neointimal smooth muscle proliferation. Disclosures of the above references are hereby incorporated in their entireties.

Anti-thrombotic agents useful in combination with the compositions of the invention include, for example, inhibitors of the IIb/IIIa integrin; tissue factor inhibitors; and anti-thrombin agents. An antiarrhythmic agent, such as a local anesthetic (class I agent), sympathetic antagonist (class II agent), antifibrillatory agent (class III agent) calcium channel agent (class IV agent) or anion antagonist (class V agent) as described in Vukmir, Am. J. Emer. Med. 13:459-470 (1995); Grant, PACE 20:432-444 (1997); Assmann I., Curr. Med. Res. Opin. 13:325-343 (1995); and Lipka et al., Am. Heart J. 130:632-640 (1995), disclosures of which are hereby incorporated by reference in their entireties, may also be used. Examples of class I agents include: procainamide; quinidine or disopyramide; lidocaine; phenyloin; tocainide or mexiletine; encainide; flecainide; lorcainide; propafenone (III) or moricizine. Sympathetic antagonists include: propranolol, esmolol, metoprolol, atenelal, or acebutolol. Examples of antifibrillatory agents are bretylium, amiodarone, sotalol (II) or N-acetylprocainamide. Class IV agents include verapamil, diltiazer, and bepridil, and anion antagonists such as alinidine.

Congestive heart failure therapeutic agents include TNF inhibitors such as Embrel™ (Immunex Corp.; Seattle, Wash.), TBC11251, or an ACE (angiotensin converting enzyme) inhibitor, such as Natrecor (nesiritide; Scios, Inc.). Angiogenic agents, for example, recombinant VEGF isoforms, such as rhVEGF developed by Genentech; a nucleic acid molecule encoding the 121 amino acid isoform of VEGF (BioByPass™; GenVec/Parke Davis); or a nucleic acid encoding VEGF-2 (Vascular Genetics, Inc.); FIBLAST™, a recombinant form of FGF-2 being developed by Scios, Inc. (Mountain View, Calif.) and Wyeth Ayerst Laboratories (Radnor, Pa.), GENERX™, or an adenoviral gene therapy vector encoding FGF-4 developed by Collateral Therapeutics (San Diego, Calif.) and Schering A G (see Miller and Abrams, Gen. Engin. News 18:1 (1998), disclosure of which is hereby incorporated by reference in its entirety), are also useful in combination with the CPP-related compositions of the invention. Finally, calcium antagonists, such as amlodipine (Marche et al., Int. J. Cardiol. 62 (Suppl.):S17-S22 (1997); Schachter, Int. J. Cardiol. 62 (Suppl.):S85-S90 (1997)); nicardipine; nifedipine; propanolol; isosorbide dinitrate; diltiazem; and isradipine (Nayler (Ed.) Calcium Antagonists pages 157-260 London: Academic Press (1988); Schachter, Int. J. Cardiol. 62 (Suppl.):S9-S15 (1997)) are also advantageous therapeutic agents for cardiovascular disorders.

In one aspect of the invention, the CPPs, CPP modulators, and anti-CPP antibodies of the invention can be used in a drug-eluting stent, for preventing and treating internal thickening or restenosis that occurs after injury due to stenting. Coronary artery stenting is the use of tiny mesh scaffolding devices to prop open clogged heart arteries, which is used in 80% of patients who undergo balloon angioplasty. Among patients undergoing angioplasty, ca. 30 to 50% will develop restenosis within six months if they did not have a stent, and ca. 20 to 30% of those who have successful stent implantation develop in-stent restenosis within six months. In order to reduce the occurrence of restenosis, coated stents have been developed, for example with heparin coating to decrease the thrombogenicity of the stent surface (for a review, see Kocsis J F et al., J. Long Term Eff. Med. Implants, 2000, 10(1-2), p. 19-45) and more recently have appeared drug-eluting stents, which allow for the release of a particular drug at the stent implantation site (for a review, see Chong P H and Cheng J W, Ann. Pharmacother., 2004, 38(4):661-669).

For the purpose of using them in the coating of stents, the compositions of the invention need to be prepared into a suitable pharmaceutical delivery form, for example as described above in the section entitled Pharmaceutical Compositions, in order to be stable over a long period of time at body temperature and to provide a slow release of the compositions of the invention at the local site of stenting. Other exemplary methods for the preparation of stents coated with compositions of the invention are known to one skilled in the art, and can be found for example in International Patent Application WO04/002549, the disclosure of which is incorporated herein by reference in its entirety.

An advantage of using the compositions of the invention as coating material for stents is that the composition is applied to the vessel at the precise site and at the time of vessel injury. This kind of local drug administration can be used to achieve higher tissue concentrations of the drug without the risk of systemic toxicity.

Having generally described this invention, a further understanding can be obtained by reference to certain specific examples which are provided herein for purposes of illustration only, and are not intended to be limiting unless otherwise specified.

### EXAMPLES

### Example 1

### Characterization of CPP Levels in Disease and Control Populations

Subjects enrolled in the Duke Databank for Cardiovascular Disease were selected on the basis of coronary artery disease (CAD). A total of 241 CAD patients and control individuals were further matched for gender, age, and ethnicity and individuals with plasma abnormalities were excluded. A set of 53 CAD patients and a set of 53 control individuals were established. Six liters of plasma were pooled from each set. An aliquot of plasma was retained from each individual, thus allowing a positive result in the pooled sample to be confirmed for each member of the population. Such confirmation is valuable to erase possible confounding effects of an individual with an aberrant level of a specific polypeptide that is not related to a cardiovascular disorder. Two and a half liters of pooled plasma from each population was subjected to separation by multiple chromatography steps according to the Microprot® process as follows:

Step 1: HSA/IgG Depletion

125 ml frozen plasma were defrost and filtered on 0.45 μm sterile filter in a sterile hood.

Filtrate was injected on two inline columns of respectively 300 ml of HSA ligand Sepharose fast Flow column (Amersham, Upsala, Sweden), 5 cm ID, 15 cm length; and 100 ml Protein G Sepharose fast Flow column (Amersham, Upsala, Sweden), 5 cm ID, 5 cm length.

Columns were equilibrated and washed with 50 mM PO4 buffer, pH 7.1, 0.15M NaCl. Flow rate was 5 ml/min.

Non-retained fraction (350 ml) was frozen until second step. Twenty runs were performed.

Step 2: Gel Filtration/Reverse Phase Capture Step

Sample from step 1 was defrosted and filtered on 0.45 μm sterile filter in a sterile hood.

Filtrate was injected on two in line gel filtration columns: 2×9.5 litres Superdex 75 (Amersham, UK) column, 14 cm ID, 62 cm length. Column was equilibrated with 50 mM PO4 buffer pH 7.4, 0.1 M NaCl, 8M urea. Hydrophobic impurities were retained on a reverse phase precolumn: 150 ml PLRPS Polymer Labs, UK). Precolumn was switched for sample injection. Gel filtration was performed at a flow rate of 40 ml/min.

Low molecular weight proteins (<20 kDa) were oriented to in line reverse phase capture column: 50 ml PLRPS 100 angstroms Polymer labs, UK). The three-way valve controlling injection on PLRPS column was switched at a cut-off of 33 mAU (280 nm) to send gel filtration eluate into reverse phase capture column. This cut-off value was established by first using SDS-PAGE to provide an estimated range of OD values and by subsequently evaluating three cut-off values (high, median and low values of OD range). The final cut-off value was chosen to maximize the low molecular weight protein obtained, with a low molecular protein proportion of at least 85%. Low molecular weight proteins and peptides were eluted from reverse phase capture PLRPS column by one column volume gradient of 0.1% TFA, 80% CH3CN in water.

Eluate fractions (50 ml) were frozen until next step. Twenty runs were performed. At the end of this step, all reverse phase eluates were defrosted, pooled (1 liter) and shared in 7 polypropylene containers (143 ml). Containers were kept at −20° C. until use for next step.

Step 3: Cation Exchange

Sample from step 2 (147 ml) was defrosted and mixed with an equal volume of cation exchange buffer A (Gly/HCl buffer 50 mM, pH 2.7, urea 8M).

Sample was injected on a 100 ml Source 15S column (Amersham, Upsala, Sweden), 35 mm ID, 100 mm length. Column was equilibrated and washed with buffer A. Flow rate was 10 ml/min.

Proteins and peptides were eluted with step gradient from 100% buffer A until 100% buffer B (buffer A containing 1M NaCl):

3 column volumes 7.5% B (75 mM NaCl)

3 column volumes 10% B (100 mM NaCl)

3 column volumes 17.5% B (175 mM NaCl)

2 column volumes 22.5% B (225 mM NaCl)

2 column volumes 27.5% B (275 mM NaCl)

2 column volumes 100% B (1 M NaCl)

45 to 60 fractions were collected based on peak. Seven runs were conducted. After 7 runs were achieved, fractions were pooled intra and inter run in order to obtain 18 fractions. Fractions were kept at −20° C. until use for next step.

Step 4: Reduction/Alkylation and Reverse Phase HPLC Fractionation 1

After adjusting the pH to 8.5 with concentrated Tris-HCl, each of the 18 cation exchange fractions was reduced with dithioerythritol (DTE, 30 mM, 3 hours at 37° C.) and alkylated with iodoacetamid (120 mM, 1 hour 25° C. in the dark). The latter reaction was stopped with the addition of DTE (30 mM) followed by acidification (TFA, 0.1%). The fractions were then injected on an Uptispher C8, 5 um, 300 angstroms column (Interchim, France), 21 mm ID, 150 mm length. Injection was performed with a 10 ml/min flow rate.

C8 column was equilibrated and washed with 0.1% TFA in water (solution A). Proteins and peptides were eluted with a biphasic gradient from 100% A until 100% B (0.1% TFA, 80% CH3CN in water) in 60 min. Flow rate was 20 ml/min. Thirty fractions of 40 ml were collected.

Based on the measured optical density (OD) at 280 nm of each fraction, which reflects the protein concentration in that fraction, aliquots of similar protein content were created for each fraction.

All aliquots were frozen and kept for further use except one per fraction which was dried with a Speed Vac (Savant, Fischer, Geneva) after addition of 500 ul 10% glycerol in water in each fraction, in order to prevent excess drying. Dried fractions were kept at −20° C. until use for next step.

Step 5: Reverse Phase HPLC Fractionation 2

Dried samples from step 4 were resuspended in 1 ml of solution A (0.03% TFA in water) and injected on a Vydac LCMS C4 column, 5 micrometers, 300 angstroms (Vydac, USA), 4.6 mm ID, 150 mm length. Flow rate was 0.8 ml/min.

C4 column was equilibrated and washed with solution A and proteins and peptides were eluted with a biphasic gradient adapted to elution position of the sample in Reverse Phase HPLC Fractionation 1. Intact mass data were acquired using Electrospray Ion Trap Mass spectrometry. Sixteen different gradients were used with a CH3CN concentration range minus and plus 5% CH3CN of RP1 fraction corresponding solvent concentration. For proteins eluted in RP1 with a solvent concentration equal to or greater than 30% CH3CN, the starting elution conditions for the RP2 gradient was set, in CH3CN percentage, at the RP1 elution concentration minus 30%. Twenty-four eluted fractions were collected in a deep well plate, adopting optimized different collection configurations designed for optimal SpeedVac concentration and further robotic treatment.

Step 6: Mass Detection

About 13,000 fractions were collected following reverse phase HPLC fractionation 2 into 96-well deep well plates (DWP). A small proportion (2.5%) of the volume was diverted to online analysis using LC-ESI-MS (Bruker Esquire). Aliquots of undigested proteins were mixed with MALDI matrices, and spotted on MALDI plates together with mass calibration standards and sensitivity standards. Automated spotting devices (Bruker MALDI sample prep. Robots) were used. Two different MALDI matrices were employed: sinapic acid (SA), also known as sinapinic acid, trans-3,5-dimethoxy-4-hydroxycinnamic acid, and alpha-cyano-4-hydroxycinnamic acid (HCCA). MALDI plates were subjected to mass detection using Bruker Reflex III MALDI MS apparati. The 96-well plates were stored at +4 C.

96-well plates (DWP) were recovered and subjected to two sequential concentration steps. Volumes were concentrated from 0.8 ml to about 50 microl per well by drying with a SpeedVac, and then resolubilized to ca. 200 microl and reconcentrated to about 50 microl per well, and stored at +4 C. Proteins were then digested by re-buffering, adding trypsin to the wells, sealing and incubating the plates at 37 C for 12 hours, followed by quenching (addition of formic acid to bring the pH down to 2.0). The concentration of trypsin to be added to the wells was adjusted based on the OD at 280 mm recorded for each particular fraction. This ensured an optimal use of trypsin and a complete digestion of the most concentrated fractions. Automated spotting devices (Bruker MALDI sample prep. Robots) were used to deposit a volume from each well, pre-mixed with a HCCA matrix onto a MALDI plate together with sensitivity and mass calibration standards. MALDI plates were analyzed using a Bruker Reflex III MALDI MS device. Contents from each well of the 96 well plates were analyzed with LC-ESI-MS-MS Bruker Esquire ESI Ion-Trap MS devices.

Step 7: Detection and Identification of Low Abundance Peptides in Human Plasma

Separated fractions are further subjected to mass spectrometry (both matrix-assisted laser desorption/ionization (MALDI) and MS-MS) for separation and detection.

Intact mass data, Peptide Mass Fingerprints and peptide sequence data were integrated for protein identification and characterization. Proteins were identified using Mascot software (Matrix Science Ltd., London, UK), and results from peptide identification were checked by manual analysis of the spectra.

Among the proteins identified by this process, Calgranulin A (S100 calcium-binding protein A8, of SwissProt accession number P05109), was found to be expressed to a greater extent in the pooled sample from controls than in the pooled sample from CAD patients (e.g., peptides from the protein were observed in twice as many control fractions compared with disease fractions, and the cumulated scores obtained during mass spectra identification of this protein were 2.5-fold higher for the control sample). Calgranulin A has been characterized as a pro-inflammatory protein (Odink, et al., Nature 330 (6143), 80-82 (1987) and numerous later references). It is expressed by extravasating myeloid cells during inflammatory responses, where it binds to a glycosaminoglycan structure on epithelial cells (Robinson, et al., JBC 277:3658-65 (2002)). Interestingly, PCT publication WO 00/61742 discloses the use of Calgranulin A for the treatment of cardiac insufficiency, e.g. caused by arteriosclerosis. Moreover, PCT publication WO 00/18970 discloses the use of Calgranulin A as an inhibitor of vascular membrane growth for prevention of myocardial infarction and hypertension. It appears therefore that the protein separation and identification approach described herein is efficient at providing proteins which, when detected at higher levels in the control sample than in the disease sample, have a beneficiary effect for the treatment of the studied disease.

Conversely, the methods of protein separation and identification described in this Example have allowed the identification of the Matrix Gla Protein (of SwissProt accession number P08493) as overexpressed in the pooled sample from CAD patients by comparison with the pooled sample from controls (e.g., peptides from the protein were observed in almost twice as many disease fractions compared with control fractions, and the cumulated scores obtained during mass spectra identification of this protein were 2-fold higher for the disease sample). MGP is a vitamin K-dependent protein which associates with the organic matrix of bone and cartilage. Mori, et al. demonstrated that MGP is capable of inhibiting vascular calcification (FEBS Letters 433:19-22 (1998)). MGP levels are increased in atherosclerotic plaques as a likely feedback response to vessel calcification. PCT publications WO 01/02863 and WO 01/25427 describe MGP as a biomarker for atherosclerosis and cardiovascular disorders. It appears therefore that the protein separation and identification approach described herein is efficient at providing proteins which have a recognized use in the diagnosis of the studied disease.

Finally, the tryptic peptides of SEQ ID NOs:4-6, listed in FIG. 1 and Table 1, were observed by tandem mass spectrometry at a 9.7-fold higher level in the Coronary Artery Disease sample. The presence of a tryptic peptide indicates that a polypeptide comprising the amino acid sequence of SEQ ID NO:4 (TPVCTTGQGSGSTATVFAMAELQK), SEQ ID NO:5 (VWFELTQGSITK), or SEQ ID NO:6 (SLSGTAFGGFLMFK) was present at a higher level in the starting plasma sample from individuals with CAD. Such polypeptides include those represented by the sequences of SEQ ID NOs:1 and 2, and in particular, SEQ ID NO:3 (CPP 22). The sequences of the peptides, along with those observed by MALDI mass spectrometry, define the CPP peptides of the invention, SEQ ID NOs:1-6.

### Example 2

### Construction of a New Splice Variant of EndoGlyx-1

The amino acid sequences of the tryptic peptides identified as detailed above in Example 1 match to the C-terminal part of the protein described by Christian et al. as EndoGlyx-1 (J. Biol. Chem. 276:48588-48595 (2001)). Since the methods of the invention, however, detect proteins with a molecular weight, on average, below 20 kDa (see Example 1, Step 2), it is not possible that the tryptic peptides listed in Example 1 have been released by the species described by Christian et al. (all of them above 100 kDa in molecular weight). The proteolytic product of SEQ ID NO:3 is the most likely species circulating in the plasma of the subjects under study.

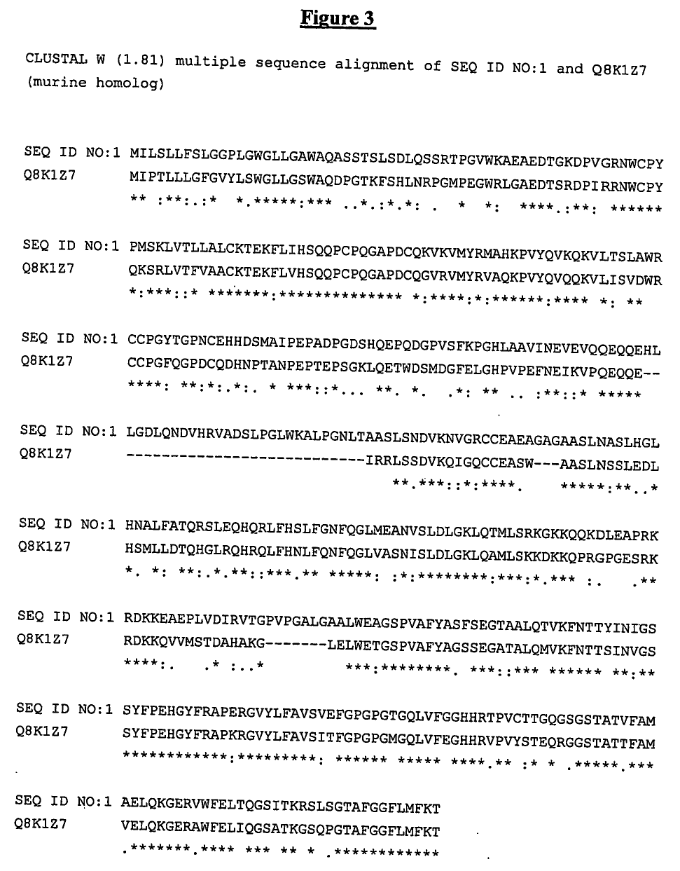

Examination of the mouse orthologue of the EndoGlyx-1 protein, however, allows the construction of an alternative full length protein from the EndoGlyx-1 gene, with the resulting DNA sequence of SEQ ID NO:7 and the precursor amino acid sequence of SEQ ID NO:1 (see FIG. 3 for an alignment of the mouse orthologue with the new human splice variant of SEQ ID NO:1). The mature form of the new splice variant is presented in SEQ ID NO:2.

The DNA sequence for the new human splice variant (SEQ ID NO:7) was constructed using the exons 1, 2, 3, 4, 5′, 6′ and 7 described in the Table 2 below, using the following public human genomic DNA: AC025268.8 (PRI 09-MAY-2002), available from NCBI (http://www.ncbi.nlm.nih.gov). This entry has the following definition: “Homo sapiens chromosome 10 clone RP11-411A19, complete sequence”, and a length of 149668 bp.

The EndoGlyx-1 protein, on the other hand, is constructed from exons 1, 2, 3, 4, 5, 6 and 7 from Table 2 below, with a total length of 949 amino acids, to be compared with 456 amino acids for the new splice variant of SEQ ID NO:1.

TABLE 2

| Positive strand | start | end |
|---|---|---|
| exon 1 | 58986 | 59149 |
| exon 2 | 70838 | 70966 |
| exon 3 | 71051 | 71157 |
| exon 4 | 71259 | 71339 |
| exon 5′ | 72092 | 72237 |
| exon 5 | 72092 | 72265 |
| exon 6 | 72399 | 74210 |
| exon 6′ | 73850 | 74210 |
| exon 7 | 79402 | 79784 |

### Example 3

### Chemical Synthesis of CPPs

In this example, a CPP of the invention is synthesized. Peptide fragment intermediates are first synthesized and then assembled into the desired polypeptide.

A CPP can initially be prepared in, e.g. 5 fragments, selected to have a Cys residue at the N-terminus of the fragment to be coupled. Fragment 1 is initially coupled to fragment 2 to give a first product, then after preparative HPLC purification, the first product is coupled to fragment 3 to give a second product. After preparative HPLC purification, the second product is coupled to fragment 4 to give a third product. Finally, after preparative HPLC purification, the third product is coupled to fragment 5 to give the desired polypeptide, which is purified and refolded.

Thioester Formation

Fragments 2, 3, 4, and 5 are synthesized on a thioester generating resin, as described above. For this purpose the following resin is prepared: S-acetylthioglycolic acid pentafluorophenylester is coupled to a Leu-PAM resin under conditions essentially as described by Hackeng et al (1999). In the first case, the resulting resin is used as a starting resin for peptide chain elongation on a 0.2 mmol scale after removal of the acetyl protecting group with a 30 min treatment with 10% mercaptoethanol, 10% piperidine in DMF. The N^{α} of the N-terminal Cys residues of fragments 2 through 5 are protected by coupling a Boc-thioproline (Boc—SPr, i.e. Boc-L-thioproline) to the terminus of the respective chains instead of a Cys having conventional N⁺ or S^{β} protection, e.g. Brik et al, J. Org. Chem., 65: 3829-3835 (2000).

Peptide Synthesis

Solid-phase synthesis is performed on a custom-modified 433A peptide synthesizer from Applied Biosystems, using in situ neutralization/2-(1H-benzotriazol-1-yl)-1,1,1,3,3-tetramethyluronium hexafluoro-phosphate (HBTU) activation protocols for stepwise Boc chemistry chain elongation, as described by Schnolzer et al, Int. J. Peptide Protein Res., 40: 180-193 (1992). Each synthetic cycle consists of N^{α}—Boc-removal by a 1 to 2 min treatment with neat TFA, a 1-min DMF flow wash, a 10-min coupling time with 2.0 mmol of preactivated Boc-amino acid in the presence of excess DIEA and a second DMF flow wash. Nα-Boc-amino acids (2 mmol) are preactivated for 3 min with 1.8 mmol HBTU (0.5M in DMF) in the presence of excess DIEA (6 mmol). After coupling of Gln residues, a dichloromethane flow wash is used before and after deprotection using TFA, to prevent possible high temperature (TFA/DMF)-catalyzed pyrrolidone carboxylic acid formation. Side-chain protected amino acids are Boc-Arg(p-toluenesulfonyl)-OH, Boc-Asn(xanthyl)-OH, Boc-Asp(O-cyclohexyl)-OH, Boc-Cys(4-methylbenzyl)-OH, Boc-Glu(O-cyclohexyl)-OH, Boc-His(dinitrophenylbenzyl)-OH, Boc-Lys(2-Cl-Z)-OH, Boc-Ser(benzyl)-OH, Boc-Thr(benzyl)-OH, Boc-Trp(cyclohexylcarbonyl)-OH and Boc-Tyr(2-Br-Z)-OH (Orpagen Pharma, Heidelberg, Germany). Other amino acids are used without side chain protection. C-terminal Fragment 1 is synthesized on Boc-Leu-O—CH₂-Pam resin (0.71 mmol/g of loaded resin), while for Fragments 2 through 5 machine-assisted synthesis is started on the Boc-Xaa-S—CH₂—CO-Leu-Pam resin. This resin is obtained by the coupling of S-acetylthioglycolic acid pentafluorophenylester to a Leu-PAM resin under standard conditions. The resulting resin is used as a starting resin for peptide chain elongation on a 0.2 mmol scale after removal of the acetyl protecting group with a 30 min treatment with 10% mercaptoethanol, 10% piperidine in DMF.

After chain assembly is completed, the peptide fragments are deprotected and cleaved from the resin by treatment with anhydrous hydrogen fluoride for 1 hr at 0° C. with 5% p-cresol as a scavenger. In all cases except Fragment 1, the imidazole side chain 2,4-dinitrophenyl (DNP) protecting groups remain on His residues because the DNP-removal procedure is incompatible with C-terminal thioester groups. However DNP is gradually removed by thiols during the ligation reaction, yielding unprotected His. After cleavage, peptide fragments are precipitated with ice-old diethylether, dissolved in aqueous acetonitrile and lyophilized. The peptide fragments are purified by RP-HPLC with a C18 column from Waters by using linear gradients of buffer B (acetonitrile/0.1% trifluoroacetic acid) in buffer A (H₂O/0.1% trifluoroacetic acid) and UV detection at 214 nm. Samples are analyzed by electrospray mass spectrometry (ESMS) using an Esquire instrument (Brücker, Bremen, Germany), or like instrument.

Native Chemical Ligations

As described more fully below, the ligation of unprotected fragments is performed as follows: the dry peptides are dissolved in equimolar amounts in 6M guanidine hydrochloride (GuHCl), 0.2M phosphate, pH 7.5 in order to get a final peptide concentration of 1-8 mM at a pH around 7, and 1% benzylmercaptan, 1% thiophenol is added. Usually, the reaction is carried out overnight and is monitored by HPLC and electrospray mass spectrometry. The ligation product is subsequently treated to remove protecting groups still present. Opening of the N-terminal thiazolidine ring further required the addition of solid methoxamine to a 0.5M final concentration at pH3.5 and a further incubation for 2 h at 37° C. A 10-fold excess of Tris(2-carboxyethyl)phosphine is added before preparative HPLC purification. Fractions containing the polypeptide chain are identified by ESMS, pooled and lyophilized.

The ligation of fragments 4 and 5 is performed at pH7.0 in 6 M GuHCl. The concentration of each reactant is 8 mM, and 1% benzylmercaptan and 1% thiophenol were added to create a reducing environment and to facilitate the ligation reaction. An almost quantitative ligation reaction is observed after overnight stirring at 37° C. At this point in the reaction, CH₃—O—NH₂.HCl is added to the solution to get a 0.5M final concentration, and the pH adjusted to 3.5 in order to open the N-terminal thiazolidine ring. After 2 h incubation at 37° C., ESMS is used to confirm the completion of the reaction. The reaction mixture is subsequently treated with a 10-fold excess of Tris(2-carboxyethylphosphine) over the peptide fragment and after 15 min, the ligation product is purified using the preparative HPLC (e.g., C4, 20-60% CH₃CN, 0.5% per min), lyophilized, and stored at −20° C.

The same procedure is repeated for the remaining ligations with slight modifications.

Polypeptide Folding

The full length peptide is refolded by air oxidation by dissolving the reduced lyophilized protein (about 0.1 mg/mL) in 1M GuHCl, 100 mM Tris, 10 mM methionine, pH 8.6 After gentle stirring overnight, the protein solution is purified by RP-HPLC as described above.

### Example 4

### Preparation of CPP Antibody Compositions

Substantially pure CPP or a portion thereof is obtained. The concentration of protein in the final preparation is adjusted, for example, by concentration on an Amicon filter device, to the level of a few micrograms per ml. Monoclonal or polyclonal antibodies to the protein are then prepared as described in the sections titled “Monoclonal antibodies” and “Polyclonal antibodies.”

Briefly, to produce an anti-CPP monoclonal antibody, a mouse is repetitively inoculated with a few micrograms of the CPP or a portion thereof over a period of a few weeks. The mouse is then sacrificed, and the antibody producing cells of the spleen isolated. The spleen cells are fused by means of polyethylene glycol with mouse myeloma cells, and the excess unfused cells destroyed by growth of the system on selective media comprising aminopterin (HAT media). The successfully fused cells are diluted and aliquots of the dilution placed in wells of a microtiter plate where growth of the culture is continued. Antibody-producing clones are identified by detection of antibody in the supernatant fluid of the wells by immunoassay procedures, such as ELISA, as originally described by Engvall, E., Meth. Enzymol. 70: 419 (1980), the disclosure of which is incorporated herein by reference in its entirety. Selected positive clones can be expanded and their monoclonal antibody product harvested for use. Detailed procedures for monoclonal antibody production are described in Davis, L. et al. Basic Methods in Molecular Biology Elsevier, New York. Section 21-2, the disclosure of which is incorporated herein by reference in its entirety.

For polyclonal antibody production by immunization, polyclonal antiserum containing antibodies to heterogeneous epitopes in the CPP or a portion thereof are prepared by immunizing a mouse with the CPP or a portion thereof, which can be unmodified or modified to enhance immunogenicity. Any suitable nonhuman animal, preferably a non-human mammal, may be selected including rat, rabbit, goat, or horse.

Antibody preparations prepared according to either the monoclonal or the polyclonal protocol are useful in quantitative immunoassays which determine concentrations of CPP in biological samples; or they are also used semi-quantitatively or qualitatively to identify the presence of antigen in a biological sample. The antibodies may also be used in therapeutic compositions for killing cells expressing the protein or reducing the levels of the protein in the body.

1. A method of screening for and/or diagnosis of a cardiovascular disorder in a subject, comprising the steps of:
a) detecting and/or quantifying the level of a polypeptide in a biological sample from said subject, wherein the polypeptide is selected from:
i) a polypeptide comprising the amino acid sequence of SEQ ID NO:3;
ii) a variant, with at least 75% sequence identity, having one or more amino acid substitutions, deletions or insertions relative to the amino acid sequence shown in SEQ ID NO: 3; and
iii) a fragment of a polypeptide as defined in i) or ii) above which is a least ten amino acids long, and
b) comparing said level to that of a control sample,
wherein an increase in said level relative to that of the control is indicative of a cardiovascular disorder.

2. A method of predicting a cardiovascular disorder in a subject, comprising the steps of:
a) detecting and for quantifying the level of a polypeptide in a biological sample from said subject, wherein the polypeptide is selected from:
i) a polypeptide comprising the amino acid sequence of SEQ ID NO:3,
ii) a variant, with at least 75% sequence identity, having one or more amino acid substitutions, deletions or insertions relative to the amino acid sequence shown in SEQ ID NO: 3; and
iii) a fragment of a polypeptide as defined in i) or ii) above which is a least ten amino acids long, and
b) comparing said level to that of a control sample,
wherein an increase in said level relative to that of the control indicates a risk of developing a cardiovascular disorder.

3. The method of claim 1, wherein said cardiovascular disorder is Coronary Artery Disease (CAD).

4. The method of claim 1, wherein said biological sample is plasma.

5. The method of claim 1, wherein said polypeptide is detected and/or quantified by mass spectrometry.

6. The method of claim 1, wherein said polypeptide is detected and for quantified by Enzyme-Linked Immuno Sorbent Assay.

7. The method of claim 1, wherein said detecting and/or quantifying the level of a polypeptide in a biological sample is performed ex vivo.

8. An isolated polypeptide comprising an amino acid sequence selected from the group consisting of SEQ ID NOs: 1-6.

9. The polypeptide of claim 8, wherein said polypeptide is fused to a heterologous polypeptide sequence.

10. An anti-Cardiovascular disorder Plasma Polypeptide antibody that selectively binds to a polypeptide comprising the amino acid sequence selected from the group consisting of SEQ ID NOs: 1-6.

11. A method of binding an antibody to a Cardiovascular disorder Plasma Polypeptide comprising the steps of:
i) contacting the antibody of claim 9 with a biological sample under conditions that permit antibody binding; and
ii) removing contaminants.

12. The method of claim 11, wherein said antibody is attached to a label group.

13. The method of claim 11, wherein said sample is human plasma.

14. The use of at least one polypeptide selected from:
i) a polypeptide comprising the amino acid sequence selected from the group consisting of SEQ ID NOs: 1 to 3,
ii) a variant, with at least 75% sequence identity, having one or more amino acid substitutions, deletions or insertions relative to the amino acid sequence shown in SEQ ID NOs:1 to 3; and
iii) a fragment of a polypeptide as defined in i) or ii) above which is a least ten amino acids long,
in the preparation of a medicament for the prophylaxis and/or treatment of cardiovascular disorders or in the preparation of a drug-eluting stent.

15. The use of an antibody from claim 10 in the preparation of a medicament for the prophylaxis and/or treatment of cardiovascular disorders or in the preparation of a drug-eluting stent.

16. A method of identifying a Cardiovascular disorder Plasma Polypeptide modulator comprising the steps of:
i) contacting a test compound with a polypeptide selected from the group consisting of SEQ ID NOs: 1-6 under sample conditions permissive for at least one CPP biological activity,
ii) determining the level of said at least one CPP biological activity;
iii) comparing said level to that of a control sample lacking said test compound, and
iv) selecting a test compound which causes said level to change for further testing as a CPP modulator for the prophylactic and/or therapeutic treatment of cardiovascular disorders.

17. An isolated polynucleotide comprising the sequence of SEQ ID NO:7.

18. A method of identifying a modulator of a cardiovascular disorder comprising the steps of:
(a) administering a candidate agent to a non-human test animal which is predisposed to be affected or which is affected by the cardiovascular disorder,
(b) administering the candidate agent of (a) to a matched control non-human animal not predisposed to be affected or not being affected by the cardiovascular disorder;
(c) detecting and for quantifying the level of a polypeptide in a biological sample obtained from the non-human test animal of step (a) and from the control animal of step (b), wherein the polypeptide is selected from:
i) a polypeptide comprising the amino acid sequence of SEQ ID NO: 3;
ii) a variant, with at least 75% sequence identity, having one or more amino acid substitutions, deletions or insertions relative to the amino acid sequence shown in SEQ ID NO: 3, and
iii) a fragment of a polypeptide as defined in i) or ii) above which is a least ten amino acids long, and
(d) comparing the levels of the polypeptide of step (c); wherein a displacement of the level of the polypeptide in the biological sample obtained from the non-human test animal towards the level of the polypeptide in the biological sample obtained from the control animal indicates that the candidate agent is a modulator of the cardiovascular disorder.

19. The method of claim 18, wherein the non-human test animal which is predisposed to be affected or which is affected by the cardiovascular disorder comprises an increased plasma level of a polypeptide selected from:
i) a polypeptide comprising the amino acid sequence of SEQ ID NO: 3;
ii) a variant, with at least 75% sequence identity, having one or more amino acid substitutions, deletions or insertions relative to the amino acid sequence shown in SEQ ID NO: 3, and
iii) a fragment of a polypeptide as deemed in i) or ii) above which is a least ten amino acids long.

20. A method for monitoring the efficacy of a treatment of a subject having or at risk of developing a cardiovascular disorder with an agent, the method comprising:
(a) obtaining a pre-administration biological sample from the subject prior to administration of the agent;
(b) detecting and/or quantifying the level of a polypeptide in the biological sample from said subject, wherein the polypeptide is selected from:
i) a polypeptide comprising the amino acid sequence of SEQ ID NO: 3;
ii) a variant, with at least 75% sequence identity, having one or more amino acid substitutions, deletions or insertions relative to the amino acid sequence shown in SEQ ID NO: 3; and
iii) a fragment of a polypeptide as defined in i) or ii) above which is a least ten amino acids long; and
(c) obtaining one or more post-administration biological samples from the subject;
(d) detecting the level of the polypeptide in the post-administration sample or samples;
(e) comparing the level of the polypeptide m the pre-administration sample with the level of the polypeptide in the post-administration sample; and
(f) adjusting the administration of the agent accordingly.
